(19) United States
(12) Patent Application Publication
(10) Pub. No.: US 2021/0396755 A1
(43) Pub. Date: Dec. 23, 2021

(54) RAPID MULTIPLEXED SEROLOGICAL TEST

(71) Applicant: Genalyte, Inc., San Diego, CA (US)

(72) Inventors: Lawrence Cary Gunn, III, Encinitas, CA (US); Richard Deane Hockett, JR., Carlsbad, CA (US); Sasi Mudumba, San Diego, CA (US)

(21) Appl. No.: 17/220,888

(22) Filed: Apr. 1, 2021

Related U.S. Application Data
(60) Provisional application No. 63/007,315, filed on Apr. 8, 2020.
(60) Provisional application No. 63/005,112, filed on Apr. 3, 2020.
(60) Provisional application No. 63/004,439, filed on Apr. 2, 2020.

(51) Int. Cl. G01N 33/569; B01L 3/00; G01N 21/39

(52) U.S. Cl. CPC G01N 33/56983; G06N 20/00; G01N 21/39; B01L 3/502715

(57) ABSTRACT

Disclosed herein are methods of performing multiplexed serological immunoassays to detect multiple antigens in parallel to determine if a patient has an infection or an immune disorder. Use of multiple antigens in parallel increases specificity and/or sensitivity towards assaying the infection or immune disorder. The infection may be a viral infection such as a SARS-CoV-2 viral infection, a variant of a SARS-CoV-2 viral infection, or a non-SARS-CoV-2 coronavirus infection. Also disclosed herein are methods of performing the multiplexed serological immunoassays on an optical ring resonator substrate. Also disclosed herein are methods of detecting antibodies specific for an antigen that belong to more than one immunoglobulin type.

### CROSS-RELEVANCE TO RELATED APPLICATIONS

This application claims the benefit of priority of U.S. Provisional Patent Application No. 63/004,439, filed Apr. 2, 2020, U.S. Provisional Patent Application No. 63/005,112, filed Apr. 3, 2020, and U.S. Provisional Patent Application No. 63/007,315, filed Apr. 8, 2020, each of which is hereby expressly incorporated by reference in its entirety.

### REFERENCE TO SEQUENCE LISTING

The present application is being filed along with a Sequence Listing in electronic format. The Sequence Listing is provided in a file entitled SeqListingGNLYT018A.TXT, which was created on Apr. 1, 2021 and is 41,517 bytes in size. The information in the electronic Sequence Listing is hereby expressly incorporated by reference in its entirety.

### FIELD

Aspects of the present invention relate generally to a multiplexed serological immunoassay to detect multiple antigens in parallel to determine presence or absence of an infection, such as a viral infection, or an immune disorder.

### BACKGROUND

Rapid and sensitive serological detection of antibodies specific for a particular infection or immune disorder in a biological sample of a patient is critical for quickly yet confidently determining the presence of the infection or immune disorder. In some cases, the infection is a SARS-CoV-2 viral infection, which is the causative agent of the COVID-19 coronavirus pandemic that has affected many individuals and impacted the global economy. There is a lasting need for improved and rapid serological tests for human diseases, including a SARS-CoV-2 infection. The emergence of SARS-CoV-2 variants has also led to a need for broad range tests that are able to detect these variants and/or determine the specific variant.

### SUMMARY

Disclosed herein are methods of performing multiplexed immunoassays for detecting multiple antigens in parallel. In some embodiments, the immunoassays are done on biological samples. In some embodiments, the biological samples are obtained from a subject. In some embodiments, the subject is a mammal, such as a human. In some embodiments, the biological sample is a fluid. In some embodiments, the biological sample is whole blood, plasma, or serum. In some embodiments, the biological sample is from a subject that is not infected, currently infected, previously infected, has not been previously infected, or at risk of being infected with a pathogen, such as bacteria, virus, or protozoa. In some embodiments, the pathogen is a virus. In some embodiments, the virus is a coronavirus. In some embodiments, the virus is the SARS-CoV-2 virus. In some embodiments, the virus is a non-SARS-CoV-2 virus. In some embodiments, the virus is the influenza virus. In some embodiments, the biological sample is from a subject that has an immune disorder. In some embodiments, the immune disorder is an autoimmune disease. In some embodiments, the immune disorder is cancer.

In some embodiments, the multiplexed immunoassay is performed by providing a substrate that comprises, consists essentially of, or consists of a fluidic channel. In some embodiments, a plurality of different antigens are attached to the fluidic channel at respectively different loci in the fluidic channel. These antigens are related to the infection or immune disorder and may be a peptide or nucleic acid component of an infectious agent or immune disorder. In some embodiments, the antigens are peptide fragments of a viral particle. In some embodiments, the antigens are peptide fragments of the capsid, coat, envelope, or receptor protein of a virus. In some embodiments, the biological sample is flowed through the fluidic channel under conditions that permit immunoglobulins in the biological sample to bind to an antigen attached to the fluidic channel. In some embodiments, a wash buffer is then flowed through the fluidic channel to remove any immunoglobulins that do not bind to an antigen or that bind to an antigen with weak affinity. In some embodiments, a first probe specific for a first immunoglobulin type is then flowed through the fluidic channel under conditions that permit the first probe to bind to the first immunoglobulins that are bound to the antigens attached to the fluidic channel.

In some embodiments, the multiplexed immunoassay is performed using a plurality of optical ring resonators, which may be positioned within the fluidic channel. The optical ring resonators can be used to determine changes in resonance wavelength upon contacting a plurality of antigens attached to the plurality of optical ring resonators with a biological sample, where immunoglobulins present in the biological sample will bind to one or more antigens of the plurality of antigens. The absence or presence of changes in resonance wavelength indicate the absence or presence of immunoglobulins that are specific for a unique antigen. Furthermore, by applying probes that are specific for immunoglobulin isotypes, to the bound immunoglobulins on the optical ring resonators, determining additional changes in resonance wavelength enable determination of the specific immunoglobulin type of the immunoglobulins. Using antigens with different but known sensitivities and specificities, this allows for a multiplexed immunoassay to detect one or more infections, immune disorders, or other diseases with both high sensitivity and specificity.

Embodiments of the present invention provided herein are described by way of the following numbered alternatives:

1. A method of performing a multiplexed immunoassay for detecting multiple antigens, comprising:

(a) obtaining a biological sample comprising immunoglobulins;

(b) providing a substrate comprising a fluidic channel, wherein a plurality of different antigens are attached to the fluidic channel at respectively different loci in the fluidic channel;

(c) flowing the biological sample through the fluidic channel under conditions that permit immunoglobulins in the biological sample to bind to an antigen attached to the fluidic channel;

(d) flowing a wash buffer through the fluidic channel to remove immunoglobulins that do not bind to an antigen or that bind to an antigen with weak affinity from the fluidic channel;

(e) flowing a first probe specific for a first immunoglobulin type through the fluidic channel under conditions that permit the first probe to bind to first immunoglobulins that are bound to the antigens attached to the fluidic channel.

2. The method of alternative 1, further comprising:

(f) detecting a signal indicative of the presence or absence of immunoglobulins of the first immunoglobulin type that are specific for an antigen.

3. The method of alternative 2, wherein the biological sample is from a subject and further comprising:

(g) determining, based on the presence or absence of immunoglobulins of the first immunoglobulin type that are specific for an antigen, whether or not the subject has an infection or immune disorder of interest and/or whether or not the subject has a second condition, wherein the plurality of different antigens are selected to improve the specificity and/or sensitivity for the infection or immune disorder of interest.

4. A method of performing a multiplexed immunoassay for detecting multiple antigens, comprising:

(a) obtaining a biological sample comprising immunoglobulins;

(b) providing a substrate comprising a fluidic channel and a plurality of optical ring resonators, wherein the plurality of optical ring resonators is situated within the fluidic channel, and wherein the optical ring resonators comprise multiple copies of a single antigen, wherein a plurality of different antigens are attached to different optical ring resonators;

(c) flowing the biological sample through the fluidic channel to contact the biological sample with the plurality of optical ring resonators, under conditions that permit immunoglobulins in the biological sample to bind to an antigen of an optical ring resonator;

(d) flowing a wash buffer through the fluidic channel to remove immunoglobulins that do not bind to an antigen or that bind to an antigen with weak affinity from the plurality of optical ring resonators;

(e) flowing a first probe specific for a first immunoglobulin type through the fluidic channel under conditions that permit the first probe to bind to first immunoglobulins that are bound to the antigen of one of the optical ring resonators;

(f) detecting changes in resonance wavelength for optical ring resonators during the flowing steps of at least (c) and (e), and optionally (d).

5. The method of alternative 4, further comprising:

(g) determining, based on the detected changes in resonance wavelength for the optical ring resonators, the presence or absence of immunoglobulins of the first immunoglobulin type that are specific for an antigen.

6. The method of alternative 5, wherein the biological sample is from a subject and further comprising:

(h) determining, based on the presence or absence of immunoglobulins of the first immunoglobulin type that are specific for an antigen, whether or not the subject has an infection or immune disorder, wherein the plurality of different antigens are selected to improve the specificity and/or sensitivity for the infection or immune disorder.

7. The method of any one of alternatives 4-6, wherein the plurality of optical ring resonators comprises 2, 3, 4, 5, 6, 7, 8, 9, 10, 11, 12, 13, 14, 15, 16, 17, 18, 19, 20, 21, 22, 23, 24, 25, 26, 27, or 28 optical ring resonators.

8. The method of any one of alternatives 1-7, wherein the first immunoglobulin type is IgG, IgM, IgA, IgD, or IgE.

9. The method of any one of alternatives 1-8, wherein the determining the presence or absence of immunoglobulins of the first immunoglobulin type that are specific for an antigen comprises quantitatively determining the amount of the first immunoglobulins that are specific for an antigen.

10. A method of performing a multiplexed immunoassay for detecting multiple antigens, comprising:

(a) obtaining a biological sample comprising immunoglobulins;

(b) providing a substrate comprising a fluidic channel, wherein a plurality of different antigens are attached to the fluidic channel;

(c) flowing the biological sample through the fluidic channel under conditions that permit the immunoglobulins in the biological sample to bind to an antigen attached to the fluidic channel at respectively different loci in the fluidic channel;

(d) flowing a wash buffer through the fluidic channel to remove immunoglobulins that do not bind to an antigen or that bind to an antigen with weak affinity from the loci in the fluidic channel;

(e) flowing a first probe specific for a first immunoglobulin type through the fluidic channel under conditions that permit the first probe to bind to first immunoglobulins that are bound to the antigens of the loci in the fluidic channel;

(f) flowing a second probe specific for a second immunoglobulin type through the fluidic channel under conditions that permit the second probe to bind to second immunoglobulins that are bound to the antigens of the loci in the fluidic channel.

11. The method of alternative 10, further comprising:

(g) detecting the presence or absence of immunoglobulins of the first immunoglobulin type or second immunoglobulin type that are specific for an antigen.

12. The method of alternative 11, wherein the biological sample is from a subject and further comprising:

(h) determining, based on the presence or absence of immunoglobulins of the first immunoglobulin type that are specific for an antigen, whether or not the subject has an infection or immune disorder, wherein the plurality of different antigens are selected to improve the specificity and/or sensitivity for the infection or immune disorder.

13. A method of performing a multiplexed immunoassay for detecting multiple antigens, comprising:

(a) obtaining a biological sample comprising immunoglobulins;

(b) providing a substrate comprising a fluidic channel and a plurality of optical ring resonators, wherein the plurality of optical ring resonators is situated within the fluidic channel, and wherein the optical ring resonators comprise multiple copies of a single antigen and wherein a plurality of different antigens are attached to different optical ring resonators;

(c) flowing the biological sample through the fluidic channel to contact the biological sample with the plurality of optical ring resonators, under conditions that permit the immunoglobulins in the biological sample to bind to an antigen of an optical ring resonator;

(d) flowing a wash buffer through the fluidic channel to remove immunoglobulins that do not bind to an antigen or that bind to an antigen with weak affinity from the plurality of optical ring resonators;

(e) flowing a first probe specific for a first immunoglobulin type through the fluidic channel under conditions that permit the first probe to bind to first immunoglobulins that are bound to the antigen of one of the optical ring resonators;

(f) flowing a second probe specific for a second immunoglobulin type through the fluidic channel under conditions that permit the second probe to bind to second immunoglobulins that are bound to the antigen of one of the optical ring resonators;

(g) detecting changes in resonance wavelength for optical ring resonators during the flowing steps of at least (c), (e) and (f), and optionally (d).

14. The method of alternative 13, further comprising:

(h) determining, based on the detected changes in resonance wavelength for the optical ring resonators, the presence or absence of immunoglobulins of the first immunoglobulin type or second immunoglobulin type that are specific for an antigen.

15. The method of alternative 14, wherein the biological sample is from a subject and further comprising:

(i) determining, based on the presence or absence of immunoglobulins of the first immunoglobulin type that are specific for an antigen, whether or not the subject has an infection or immune disorder, wherein the plurality of different antigens are selected to improve the specificity and/or sensitivity for the infection or immune disorder.

16. The method of any one of alternatives 13-15, wherein the plurality of optical ring resonators comprises 2, 3, 4, 5, 6, 7, 8, 9, 10, 11, 12, 13, 14, 15, 16, 17, 18, 19, 20, 21, 22, 23, 24, 25, 26, 27, or 28 optical ring resonators.

17. The method of any one of alternatives 10-16, wherein the first immunoglobulin type is IgG, IgM, IgA, IgD, or IgE, and the second immunoglobulin type is IgM, IgG, IgA, IgD, or IgE.

18. The method of any one of alternatives 10-17, wherein the determining the presence or absence of immunoglobulins of the first immunoglobulin type or second immunoglobulin type that are specific for an antigen comprises quantitatively determining the amount of the first immunoglobulins or second immunoglobulins, respectively, that are specific for an antigen.

19. The method of any one of alternatives 1-18, wherein the infection or immune disorder is a viral infection.

20. The method of alternative 19, wherein the viral infection is a coronavirus infection.

21. The method of alternative 20, wherein the coronavirus infection is a SARS-CoV-2 infection, and the plurality of antigens comprises at least one immunogenic peptide fragment of a SARS-CoV-2 protein selected from the group consisting of the S protein, M protein, N protein, E protein, and HE protein.

22. The method of alternative 19, wherein the viral infection is an influenza infection.

23. The method of any one of alternatives 1-22, wherein the biological sample is whole blood, plasma, or serum.

24. The method of any one of alternatives 1-23, wherein the biological sample is provided in a volume of 250 μL or less, such as 10, 20, 30, 40, 50, 60, 70, 80, 90, 100, 110, 120, 130, 140, 150, 160, 170, 180, 190, 200, 210, 220, 230, 240, or 250 μL, or any volume within a range defined by any two aforementioned volumes.

25. The method of any one of alternatives 1-24, wherein the method is performed within 60 minutes or less, such as 5, 10, 11, 12, 13, 14, 15, 16, 17, 18, 19, 20, 25, 30, 35, 40, 45, 50, 55, or 60 minutes, or any time duration within a range defined by any two aforementioned values.

26. The method of any one of alternatives 1-25, wherein the plurality of antigens comprises at least one antigen specific for the infection or immune disorder and at least one antigen specific for a second condition.

27. The method of alternative 28, wherein the at least one antigen specific for the infection or immune disorder is an antigen specific for SARS-CoV-2, and wherein the at least one antigen specific for a second condition is an antigen specific for a virus selected from the group consisting of non-SARS-CoV-2 coronavirus, influenza virus, and combinations thereof.

28. The method of any one of alternatives 1-27, wherein the plurality of antigens comprises at least one antigen with high specificity for an immunoglobulin associated with an infection or immune disorder and at least one antigen with high sensitivity for an immunoglobulin associated with the infection or immune disorder.

29. The method of any one of alternatives 1-28, wherein the plurality of antigens comprises two or more antigens with high specificity for an immunoglobulin associated with an infection or immune disorder and two or more antigens with high sensitivity for an immunoglobulin associated with the infection or immune disorder.

30. The method of any one of alternatives 1-29, further comprising combining the measured amount of antigens with different sensitivities for immunoglobulins associated with an infection or immune disorder and the measured amount of antigens with different specificities for immunoglobulins associated with an infection or immune disorder.

31. The method of alternative 30, wherein the combined measurements provide an overall sensitivity and specificity for an infection or immune disorder.

32. The method of alternative 28, wherein the infection or immune disorder is a SARS-CoV-2 infection and the at least one antigen with high specificity comprises at least 1, 2, 3, 4, 5, 6, 7, 8, 9, or 10 antigens with protein sequences unique to SARS-CoV-2, and the at least one antigen with high sensitivity comprises at least 1, 2, 3, 4, 5, 6, 7, 8, 9, or 10 antigens with protein sequences that are highly immunogenic but common in Coronaviridae with at least 50%, 60%, 70%, 80%, 85%, 90%, 91%, 92%, 93%, 94%, 95%, 96%, 97%, 98%, 99%, or 100% homology.

33. The method of alternative 28, wherein the infection or immune disorder is a coronavirus infection and the at least one antigen with high specificity comprises at least 1, 2, 3, 4, 5, 6, 7, 8, 9, or 10 antigens with protein sequences unique to a non-SARS-CoV-2 coronavirus, and the at least one antigen with high sensitivity comprises at least 1, 2, 3, 4, 5, 6, 7, 8, 9, or 10 antigens with protein sequences that are highly immunogenic but common in Coronaviridae with at least 50%, 60%, 70%, 80%, 85%, 90%, 91%, 92%, 93%, 94%, 95%, 96%, 97%, 98%, 99%, or 100% homology.

34. The method of alternative 32, wherein the presence of immunoglobulins that are specific for an antigen with high specificity reduces a false positive reading of a SARS-CoV-2 infection.

35. The method of alternative 32, wherein the presence of immunoglobulins that are specific for an antigen with high sensitivity reduces a false negative reading of a SARS-CoV-2 infection.

36. A method of performing a multiplexed immunoassay for detecting multiple antigens, comprising:

(a) flowing a biological sample comprising immunoglobulins from a subject through a fluidic channel of a substrate under conditions that permit immunoglobulins in the biological sample to bind to an antigen attached to the fluidic channel, wherein a plurality of different antigens are attached to the fluidic channel at respectively different loci in the fluidic channel;

(b) flowing a first probe specific for a first immunoglobulin type through the fluidic channel under conditions that permit the first probe to bind to first immunoglobulins that are bound to the antigens attached to the fluidic channel.

37. The method of alternative 36, further comprising:

flowing a wash buffer through the fluidic channel to remove immunoglobulins that do not bind to an antigen or that bind to an antigen with weak affinity from the fluidic channel after the step of (a) and before the step of (b).

38. The method of alternative 36 or 37, further comprising:

(c) detecting a signal indicative of the presence or absence of immunoglobulins of the first immunoglobulin type that are specific for an antigen.

39. The method of any one of alternatives 36-38, further comprising:

(g) determining, based on the presence or absence of immunoglobulins of the first immunoglobulin type that are specific for an antigen, whether or not the subject has an infection or immune disorder of interest and/or whether or not the subject has a second condition, wherein the plurality of different antigens are selected to improve the specificity and/or sensitivity for the infection or immune disorder of interest.

40. A method of performing a multiplexed immunoassay for detecting multiple antigens, comprising:

(a) providing a substrate comprising a fluidic channel and a plurality of optical ring resonators, wherein the plurality of optical ring resonators is situated within the fluidic channel, and wherein the optical ring resonators comprise multiple copies of a single antigen, wherein a plurality of different antigens are attached to different optical ring resonators;

(b) flowing a biological sample comprising immunoglobulins from a subject through the fluidic channel to contact the biological sample with the plurality of optical ring resonators, under conditions that permit immunoglobulins in the biological sample to bind to an antigen of an optical ring resonator;

(c) flowing a first probe specific for a first immunoglobulin type through the fluidic channel under conditions that permit the first probe to bind to first immunoglobulins that are bound to the antigen of one of the optical ring resonators;

(d) detecting changes in resonance wavelength for optical ring resonators during the flowing steps of (b)-(c).

41. The method of alternative 40, further comprising:

flowing a wash buffer through the fluidic channel to remove immunoglobulins that do not bind to an antigen or that bind to an antigen with weak affinity from the plurality of optical ring resonators after the step of (b) and before the step of (c).

42. The method of alternative 41, further comprising:

detecting changes in resonance wavelength for optical ring resonators during the flowing of the wash buffer.

43. The method of any one of alternatives 40-42, further comprising:

(e) determining, based on the detected changes in resonance wavelength for the optical ring resonators, the presence or absence of immunoglobulins of the first immunoglobulin type that are specific for an antigen.

44. The method of alternative 43, further comprising:

(f) determining, based on the presence or absence of immunoglobulins of the first immunoglobulin type that are specific for an antigen, whether or not the subject has an infection or immune disorder, wherein the plurality of different antigens are selected to improve the specificity and/or sensitivity for the infection or immune disorder.

45. The method of any one of alternatives 40-44, wherein the plurality of optical ring resonators comprises 1, 2, 3, 4, 5, 6, 7, 8, 9, 10, 11, 12, 13, 14, 15, 16, 17, 18, 19, 20, 21, 22, 23, 24, 25, 26, 27, or 28 optical ring resonators.

46. The method of any one of alternatives 36-45, wherein the first immunoglobulin type is IgG, IgM, IgA, IgD, or IgE.

47. The method of any one of alternatives 36-46, wherein the determining the presence or absence of immunoglobulins of the first immunoglobulin type that are specific for an antigen comprises quantitatively determining the amount of the first immunoglobulins that are specific for an antigen.

48. A method of performing a multiplexed immunoassay for detecting multiple antigens, comprising:

(a) flowing a biological sample comprising immunoglobulins from a subject through a fluidic channel of a substrate under conditions that permit the immunoglobulins in the biological sample to bind to an antigen attached to the fluidic channel at respectively different loci in the fluidic channel, wherein a plurality of different antigens are attached to the fluidic channel at respectively different loci in the fluidic channel;

(b) flowing a first probe specific for a first immunoglobulin type through the fluidic channel under conditions that permit the first probe to bind to first immunoglobulins that are bound to the antigens of the loci in the fluidic channel;

(c) flowing a second probe specific for a second immunoglobulin type through the fluidic channel under conditions that permit the second probe to bind to second immunoglobulins that are bound to the antigens of the loci in the fluidic channel.

49. The method of alternative 48, further comprising:

flowing a wash buffer through the fluidic channel to remove immunoglobulins that do not bind to an antigen or that bind to an antigen with weak affinity from the loci in the fluidic channel after the step of (a) and before the step of (b), and/or after the step of (b) and before the step of (c).

50. The method of alternative 48 or 49, further comprising:

(d) detecting the presence or absence of immunoglobulins of the first immunoglobulin type or second immunoglobulin type that are specific for an antigen.

51. The method of any one of alternatives 48-50, further comprising:

(e) determining, based on the presence or absence of immunoglobulins of the first immunoglobulin type that are specific for an antigen, whether or not the subject has an infection or immune disorder, wherein the plurality of different antigens are selected to improve the specificity and/or sensitivity for the infection or immune disorder.

52. A method of performing a multiplexed immunoassay for detecting multiple antigens, comprising:

(a) providing a substrate comprising a fluidic channel and a plurality of optical ring resonators, wherein the plurality of optical ring resonators is situated within the fluidic channel, and wherein the optical ring resonators comprise multiple copies of a single antigen and wherein a plurality of different antigens are attached to different optical ring resonators;

(b) flowing a biological sample comprising immunoglobulins from a subject through the fluidic channel to contact the biological sample with the plurality of optical ring resonators, under conditions that permit the immunoglobulins in the biological sample to bind to an antigen of an optical ring resonator;

(c) flowing a first probe specific for a first immunoglobulin type through the fluidic channel under conditions that permit the first probe to bind to first immunoglobulins that are bound to the antigen of one of the optical ring resonators;

(d) flowing a second probe specific for a second immunoglobulin type through the fluidic channel under conditions that permit the second probe to bind to second immunoglobulins that are bound to the antigen of one of the optical ring resonators;

(e) detecting changes in resonance wavelength for optical ring resonators during the flowing steps of (b)-(c).

53. The method of alternative 52, further comprising:

flowing a wash buffer through the fluidic channel to remove immunoglobulins that do not bind to an antigen or that bind to an antigen with weak affinity from the plurality of optical ring resonators after the step of (b) and before the step of (c), and/or after the step of (c) and before the step of (d).

54. The method of alternative 53, further comprising:

detecting changes in resonance wavelength for optical ring resonators during the flowing of the wash buffer.

55. The method of any one of alternatives 52-54, further comprising:

(f) determining, based on the detected changes in resonance wavelength for the optical ring resonators, the presence or absence of immunoglobulins of the first immunoglobulin type or second immunoglobulin type that are specific for an antigen.

56. The method of alternative 55, further comprising:

(g) determining, based on the presence or absence of immunoglobulins of the first immunoglobulin type that are specific for an antigen, whether or not the subject has an infection or immune disorder and/or whether or not the subject has a second condition, wherein the plurality of different antigens are selected to improve the specificity and/or sensitivity for the infection or immune disorder.

57. The method of any one of alternatives 52-56, wherein the plurality of optical ring resonators comprises 1, 2, 3, 4, 5, 6, 7, 8, 9, 10, 11, 12, 13, 14, 15, 16, 17, 18, 19, 20, 21, 22, 23, 24, 25, 26, 27, or 28 optical ring resonators.

58. The method of any one of alternatives 48-57, wherein the first immunoglobulin type is IgG, IgM, IgA, IgD, or IgE, and the second immunoglobulin type is IgM, IgG, IgA, IgD, or IgE.

59. The method of any one of alternatives 48-58, wherein the determining the presence or absence of immunoglobulins of the first immunoglobulin type or second immunoglobulin type that are specific for an antigen comprises quantitatively determining the amount of the first immunoglobulins or second immunoglobulins, respectively, that are specific for an antigen.

60. The method of any one of alternatives 1-59, wherein the infection or immune disorder is a viral infection.

61. The method of alternative 60, wherein the viral infection is a coronavirus infection.

62. The method of alternative 61, wherein the coronavirus infection is a SARS-CoV-2 infection, and the plurality of antigens comprises at least one immunogenic peptide fragment of a SARS-CoV-2 protein selected from the group consisting of the S protein, M protein, N protein, E protein, and HE protein.

63. The method of alternative 60, wherein the viral infection is an influenza infection.

64. The method of any one of alternatives 1-63, wherein the biological sample is whole blood, plasma, or serum.

65. The method of any one of alternatives 1-64, wherein the biological sample is provided in a volume of 250 μL or less, such as 10, 20, 30, 40, 50, 60, 70, 80, 90, 100, 110, 120, 130, 140, 150, 160, 170, 180, 190, 200, 210, 220, 230, 240, or 250 μL, or any volume within a range defined by any two aforementioned volumes.

66. The method of any one of alternatives 1-65, wherein the method is performed within 60 minutes or less, such as 5, 10, 11, 12, 13, 14, 15, 16, 17, 18, 19, 20, 25, 30, 35, 40, 45, 50, 55, or 60 minutes, or any time duration within a range defined by any two aforementioned values.

67. The method of any one of alternatives 1-66, wherein the plurality of antigens comprises at least one antigen specific for the infection or immune disorder and at least one antigen specific for a second condition.

68. The method of alternative 67, wherein the at least one antigen specific for the infection or immune disorder is an antigen specific for SARS-CoV-2, and wherein the at least one antigen specific for a second condition is an antigen specific for a virus selected from the group consisting of non-SARS-CoV-2 coronavirus, influenza virus, and combinations thereof.

69. The method of any one of alternatives 1-68, wherein the plurality of antigens comprises at least one antigen with high specificity for an immunoglobulin associated with an infection or immune disorder and at least one antigen with high sensitivity for an immunoglobulin associated with the infection or immune disorder.

70. The method of any one of alternatives 1-69, wherein the plurality of antigens comprises two or more antigens with high specificity for an immunoglobulin associated with an infection or immune disorder and two or more antigens with high sensitivity for an immunoglobulin associated with the infection or immune disorder.

71. The method of any one of alternatives 1-70, wherein the plurality of antigens comprises antigens with different sensitivities for immunoglobulins associated with an infection or immune disorder and antigens with different specificities for immunoglobulins associated with an infection or immune disorder.

72. The method of alternative 71, further comprising determining an overall sensitivity and specificity for an infection or immune disorder.

73. The method of any one of alternatives 1-72, wherein the infection or immune disorder is a SARS-CoV-2 infection and the at least one antigen with high specificity comprises at least 1, 2, 3, 4, 5, 6, 7, 8, 9, or 10 antigens with protein sequences unique to SARS-CoV-2, and the at least one antigen with high sensitivity comprises at least 1, 2, 3, 4, 5, 6, 7, 8, 9, or 10 antigens with protein sequences that are highly immunogenic but common in Coronaviridae with at least 50%, 60%, 70%, 80%, 85%, 90%, 91%, 92%, 93%, 94%, 95%, 96%, 97%, 98%, 99%, or 100% homology.

74. The method of any one of alternatives 1-73, wherein the infection or immune disorder is a coronavirus infection and the at least one antigen with high specificity comprises at least 1, 2, 3, 4, 5, 6, 7, 8, 9, or 10 antigens with protein sequences unique to a non-SARS-CoV-2 coronavirus, and the at least one antigen with high sensitivity comprises at least 1, 2, 3, 4, 5, 6, 7, 8, 9, or 10 antigens with protein sequences that are highly immunogenic but common in Coronaviridae with at least 50%, 60%, 70%, 80%, 85%, 90%, 91%, 92%, 93%, 94%, 95%, 96%, 97%, 98%, 99%, or 100% homology.

75. The method of any one of alternatives 1-74, wherein the presence of immunoglobulins that are specific for an antigen with high specificity reduces a false positive reading of a SARS-CoV-2 infection.

76. The method of any one of alternatives 1-75, wherein the presence of immunoglobulins that are specific for an antigen with high sensitivity reduces a false negative reading of a SARS-CoV-2 infection.

77. The method of any one of alternatives 21, 23-35, 62, 64-76, wherein the SARS-CoV-2 infection is caused by a SARS-CoV-2 variant.

78. The method of alternative 77, wherein the SARS-CoV-2 variant is selected from 20I/501Y.V1 (B.1.1.7), 20H/501Y.V2 (B.1.351), 20J/501Y.V3 (P.1), B.1.1.207, VUI-202102/03 (B.1.525), VUI-202101/01 (P.2), VUI-202102/01 (A.23.1), VUI 202102/04 (B.1.1.318), VUI 202103/01 (B.1.324.1), or CAL.20C (B.1.429).

79. A method of performing a multiplexed immunoassay, comprising:

(a) contacting a biological sample from a subject comprising a plurality of immunoglobulins with a plurality of optical ring resonators under conditions that permit immunoglobulins to bind to a plurality of antigens, wherein each optical ring resonator of the plurality of optical ring resonators comprises multiple copies of a single antigen, such that the plurality of optical ring resonators comprises a plurality of antigens;

(b) contacting one or more probes specific to one or more immunoglobulin types with the immunoglobulins bound to the plurality of antigens on the optical ring resonators under conditions that permit the one or more probes to bind to the immunoglobulins; and

(c) detecting changes in resonance wavelength for the plurality of optical ring resonators during the contacting step of step (a), step (b), or during both contacting steps (a) and (b).

80. The method of alternative 79, wherein a change in resonance wavelength for an individual optical ring resonator of the plurality of optical ring resonators comprising the multiple copies of the single antigen indicates that either (1) an immunoglobulin that specifically binds to the single antigen is present in the plurality of immunoglobulins, or (2) the immunoglobulin that specifically binds to the single antigen comprises an immunoglobulin type to which the one or more probes specifically bind, or (3) both (1) and (2).

81. The method of alternative 80, wherein detecting changes in resonance wavelength during the contacting step of step (a) indicates that (1) the immunoglobulin that specifically binds to the single antigen is present in the plurality of immunoglobulins.

82. The method of alternative 80 or 81, wherein detecting changes in resonance wavelength during the contacting step of step (b) indicates that (2) the immunoglobulin that specifically binds to the single antigen comprises the immunoglobulin type to which the one or more probes specifically bind.

83. The method of any one of alternatives 79-82, wherein the plurality of optical ring resonators is situated within a fluidic channel.

84. The method of alternative 83, wherein the fluidic channel is situated within a substrate or device.

85. The method of alternative 83 or 84, wherein the contacting step of step (a) comprises flowing the biological sample through the fluidic channel to contact the biological sample with the plurality of optical ring resonators and the contacting step of step (b) comprises flowing the one or more probes through the fluidic channel to contact the immunoglobulins bound to the plurality of antigens on the optical ring resonators.

86. The method of any one of alternatives 79-83, further comprising a washing step between the contacting steps of step (a) and step (b), wherein immunoglobulins that do not bind to the plurality of antigens or that bind to the plurality of antigens with weak affinity are removed from the plurality of optical ring resonators.

87. The method of alternative 85, further comprising detecting changes in resonance wavelength for the plurality of optical ring resonators during the washing step, or after the washing step and before step (b), or during both the washing step and after the washing step and before step (b).

88. The method of alternative 86 or 87, wherein the washing step comprises flowing a wash buffer through the fluidic channel to contact the wash buffer with the plurality of immunoglobulins and the plurality of optical ring resonators.

89. The method of any one of alternatives 79-88, wherein the plurality of optical ring resonators comprises 2, 3, 4, 5, 6, 7, 8, 9, 10, 11, 12, 13, 14, 15, 16, 17, 18, 19, 20, 21, 22, 23, 24, 25, 26, 27, or 28 optical ring resonators.

90. The method of any one of alternatives 79-89, wherein the one or more immunoglobulin types comprises IgG, IgM, IgA, IgD, or IgE, or any combination thereof.

91. The method of any one of alternatives 79-90, wherein the one or more immunoglobulin types comprises IgG and IgM.

92. The method of any one of alternatives 79-91, further comprising determining, based on the detected changes in resonance wavelength for the plurality of optical ring resonators, the presence or absence of immunoglobulins of the one or more immunoglobulin types that are specific for the plurality of antigens.

93. The method of alternative 92, further comprising determining, based on the presence or absence of immunoglobulins of the one or more immunoglobulin types that are specific for the plurality of antigens, whether or not the subject has or previously had an infection or immune disorder.

94. The method of alternative 93, wherein the plurality of antigens are selected to improve the specificity and/or sensitivity for detecting the infection or immune disorder.

95. The method of alternative 93 or 94, wherein the infection or immune disorder is a viral infection.

96. The method of alternative 95, wherein the viral infection is a coronavirus infection.

97. The method of alternative 96, wherein the coronavirus infection is a SARS-CoV-2 infection, and the plurality of antigens comprises at least one immunogenic peptide of a SARS-CoV-2 protein.

98. The method of alternative 97, wherein the SARS-CoV-2 protein is selected from the group consisting of the S protein, M protein, N protein, E protein, and HE protein.

99. The method of alternative 97 or 98, wherein the SARS-CoV-2 infection is caused by a SARS-CoV-2 variant.

100. The method of alternative 99, wherein the SARS-CoV-2 variant is selected from 20I/501Y.V1 (B.1.1.7), 20H/501Y.V2 (B.1.351), 20J/501Y.V3 (P.1), B.1.1.207, VUI-202102/03 (B.1.525), VUI-202101/01 (P.2), VUI-202102/01 (A.23.1), VUI 202102/04 (B.1.1.318), VUI 202103/01 (B.1.324.1), or CAL.20C (B.1.429).

101. The method of alternative 95, wherein the viral infection is an influenza infection.

102. The method of any one of alternatives 79-101, wherein the biological sample is whole blood, plasma, or serum.

103. The method of any one of alternatives 79-102, wherein the biological sample comprises a volume of 250 μL or less, such as 10, 20, 30, 40, 50, 60, 70, 80, 90, 100, 110, 120, 130, 140, 150, 160, 170, 180, 190, 200, 210, 220, 230, 240, or 250 μL, or any volume within a range defined by any two aforementioned volumes.

104. The method of any one of alternatives 79-103, wherein the method is performed within 60 minutes or less, such as 5, 10, 11, 12, 13, 14, 15, 16, 17, 18, 19, 20, 25, 30, 35, 40, 45, 50, 55, or 60 minutes, or any time duration within a range defined by any two aforementioned values.

105. The method of any one of alternatives 79-103, wherein the plurality of antigens comprises at least one antigen with high specificity for an immunoglobulin associated with the infection or immune disorder and at least one antigen with high sensitivity for an immunoglobulin associated with the infection or immune disorder.

106. The method of any one of alternatives 79-105, wherein the plurality of antigens comprises antigens associated with two or more diseases or disorders.

107. The method of alternative 106, wherein the two or more diseases or disorders comprises a SARS-CoV-2 infection, a SARS-CoV-2 variant infection, a non-SARS-CoV-2 coronavirus infection, a non-SARS-CoV-2 viral infection, influenza, or an immune disorder, or any combination thereof.

108. The method of alternative 106 or 107, wherein the plurality of antigens comprises at least one antigen with high specificity for an immunoglobulin associated with at least one of the two or more diseases or disorders and at least one antigen with high sensitivity for an immunoglobulin associated with at least one of the two or more diseases or disorders.

109. The method of any one of alternatives 106-108, further comprising determining, based on the detected changes in resonance wavelength for the plurality of optical ring resonators, an overall sensitivity and specificity for the two or more diseases or disorders.

110. The method of any one of alternatives 93-109, wherein the presence of immunoglobulins that are specific for an antigen with high specificity of the plurality of antigens reduces a false positive reading of the infection or immune disorder, or at least one of the two or more diseases or disorders.

111. The method of any one of alternatives 93-110, wherein the presence of immunoglobulins that are specific for an antigen with high sensitivity of the plurality of antigens reduces a false negative reading of the infection or immune disorder, or at least one of the two or more diseases or disorders.

112. The method of any one of alternatives 93-111, wherein the infection or immune disorder, or at least one of the two or more diseases or disorders comprises a SARS-CoV-2 infection, and the plurality of antigens comprises at least 1, 2, 3, 4, 5, 6, 7, 8, 9, or 10 antigens with protein sequences unique to SARS-CoV-2, and the plurality of antigens further comprise at least 1, 2, 3, 4, 5, 6, 7, 8, 9, or 10 antigens with protein sequences that are common in Coronaviridae with at least 50%, 60%, 70%, 80%, 90%, 91%, 92%, 93%, 94%, 95%, 96%, 97%, 98%, 99%, or 100% homology.

113. The method of any one of alternatives 93-111, wherein the infection or immune disorder, or at least one of the two or more diseases or disorders comprises a SARS-CoV-2 infection, and the plurality of antigens comprises at least 1, 2, 3, 4, 5, 6, 7, 8, 9, or 10 antigens with protein sequences unique to SARS-CoV-2, and the plurality of antigens further comprise at least 1, 2, 3, 4, 5, 6, 7, 8, 9, or 10 antigens with protein sequences that are associated with a virus that is not SARS-CoV-2.

113. The method of any one of alternatives 79-113, wherein the plurality of antigens comprise one or more of SEQ ID NOs: 1-8.

114. The method of any one of alternatives 79-114, wherein the plurality of antigens comprise one or more of SEQ ID NOs: 4-8.

115. The method of any one of alternatives 2, 5, 11, 14, 36-38, 43, 48-50, or 55, further comprising determining, based on the presence or absence of immunoglobulins of the one or more immunoglobulin types that are specific for the plurality of antigens, whether or not the subject previously had an infection or immune disorder.

116. The method of any one of alternatives 93-115, wherein determining whether or not the subject has or previously had an infection or immune disorder is performed with a machine learning algorithm.

117. The method of alternative 116, wherein the machine learning algorithm is a random forest machine learning algorithm.

### BRIEF DESCRIPTION OF THE DRAWINGS

In addition to the features described above, additional features and variations will be readily apparent from the following descriptions of the drawings and exemplary embodiments. It is to be understood that these drawings depict typical embodiments and are not intended to be limiting in scope.

FIG. 1A is a schematic block diagram of a system for detecting an analyte comprising a light source that may include a light source (e.g. a tunable light source or a broad band light source), an optical sensor, and an optical detector.

FIG. 1B illustrates a cross-section of an example optical evanescent field sensor.

FIG. 2 illustrates a perspective cross section of another example of an optical sensor having a ring resonator cavity and a coupling waveguide, formed on a silicon substrate.

FIG. 3a illustrates a top down view of another example of an optical sensor that includes a ring resonator cavity and two coupling waveguides in evanescent coupling to the ring resonator cavity.

FIGS. 3b-3d illustrate examples of non-circular shaped ring resonant cavities.

FIG. 4a illustrates a schematic of an example of a system with a fluid flow control module and a sensor array.

FIG. 4b illustrates another example of a system with a fluid flow control module and a sensor array.

FIG. 5A shows a schematic diagram of an optical sensor comprising a waveguide and a ring resonator. FIG. 5A schematically illustrates the range of wavelengths that may be input into the optical sensor and the resultant spectral output of the optical sensor. A decrease in the optical output at the resonance frequency of the ring resonator is visible in the output spectrum shown.

FIG. 5B is a perspective view of an optical sensor comprising a waveguide and a ring resonator.

FIG. 5C is a cross-section through the waveguide and ring resonator shown in FIG. 5B along the line 5-5.

FIG. 6 is a cut-away view of a waveguide schematically showing an intensity distribution having an evanescent tail extending outside the waveguide where an element such as a molecule or particle may be located so as to affect the index of refraction of the waveguide.

FIG. 7A schematically illustrates a plurality of optical sensors on a chip and an apparatus that provides light to the chip and detects light output from the chip.

FIG. 7B is a perspective view of light coupled into a waveguide on a chip using a grating coupler and light coupled out of a waveguide on a chip using a grating coupler, for example, to provide input to and collect output from an optical sensor on the chip.

FIG. 7C is a top view schematically illustrating a chip having input and output couplers connected to waveguide optical sensors comprising ring resonators. The chip further includes flow channels for flowing solution across the waveguide optical sensors and in particular the ring resonators. Input ports provide access to the flow channels. The chip further comprises identification markers to facilitate identification of the different optical sensors.

FIG. 8A depicts a stripwell for use in the immunoassay.

FIG. 8B depicts the process of adding a biological sample to the upper-right well.

FIG. 9 depicts a representative sensogram showing the results of a SARS-CoV-2 immunoassay. In the same test, IgG and IgM response can be measured sequentially.

FIG. 10 depicts peptide sequences of the SARS-CoV-2 virus S protein, M protein, E protein, and N protein.

### DETAILED DESCRIPTION

Optical sensors, such as silicon photonic microring resonators, have high spectral sensitivity towards surface binding events between an analyte of interest and an optical sensor modified with a probe for capturing the analyte of interest (i.e. a capture probe). The systems of several embodiments are based on refractive index-based sensing schemes in which the mass of bound analytes, potentially in combination with other factors such as capture probe affinity and surface density, contributes to the observed signal and measurement sensitivity.

Analytes, such as proteins, that are simultaneously low in abundance and have a lower molecular weight are often very difficult to detect. Several embodiments relate to employing an antibody to amplify the signal arising from the initial primary binding event between the analyte and capture probe. Other embodiments relate to employing a particle to further amplify the signal arising from the primary binding event and/or the signal arising from the secondary binding event of the “secondary” antibody. In certain embodiments, it is possible to improve both the sensitivity and/or the specificity of analyte detection assays, allowing for quantitative sensing in complex sample matrices.

### Definitions

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

Unless defined otherwise, all technical and scientific terms used herein have the same meaning as is commonly understood by one of ordinary skill in the art. All patents, applications, published applications and other publications referenced herein are expressly incorporated by reference in their entireties unless stated otherwise. In the event that there are a plurality of definitions for a term herein, those in this section prevail unless stated otherwise.

The articles “a” and “an” are used herein to refer to one or to more than one (for example, at least one) of the grammatical object of the article. By way of example, “an element” means one element or more than one element.

The terms “about” or “around” as used herein refer to a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

Throughout this specification, unless the context requires otherwise, the words “comprise,” “comprises,” and “comprising” will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

Unless defined otherwise, all technical and scientific terms used herein have the same meaning as is commonly understood by one of ordinary skill in the art to which this disclosure belongs. If there is a plurality of definitions for a term herein, those in this section prevail unless stated otherwise. The practice of the present disclosure will employ, unless indicated specifically to the contrary, conventional methods of molecular biology and recombinant DNA techniques within the skill of the art, many of which are described below for the purpose of illustration.

Where a range of values is provided, it is understood that the upper and lower limit, and each intervening value between the upper and lower limit of the range is encompassed within the embodiments.

The term “% w/w” or “% wt/wt” as used herein has its ordinary meaning as understood in light of the specification and refers to a percentage expressed in terms of the weight of the ingredient or agent over the total weight of the composition multiplied by 100. The term “% v/v” or “% vol/vol” as used herein has its ordinary meaning as understood in the light of the specification and refers to a percentage expressed in terms of the liquid volume of the compound, substance, ingredient, or agent over the total liquid volume of the composition multiplied by 100.

The terms “individual”, “subject”, or “patient” as used herein, means a human or a non-human mammal, e.g., a dog, a cat, a mouse, a rat, a cow, a sheep, a pig, a goat, a non-human primate, or a bird, e.g., a chicken, as well as any other vertebrate or invertebrate.

The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rodents, rats, mice, guinea pigs, or the like.

The term “biological sample” as used herein refer to any biological tissue or fluid derived from a subject such as sputum, cerebrospinal fluid, blood, blood fractions such as serum and plasma, blood cells, tissue, biopsy samples, urine, peritoneal fluid, pleural fluid, amniotic fluid, vaginal swab, skin, lymph fluid, synovial fluid, feces, tears, organs, or tumors. In some embodiments, a biological sample can include viral particles or fragments thereof, recombinant cells, cell components, cells grown in vitro, and cell culture constituents including, for example, conditioned medium resulting from the growth of cells in cell culture medium.

As used herein, the term “blood” refers to the complex fluid mixture that flows throughout the circulatory system of an organism to transport oxygen, carbon dioxide, nutrients, and waste throughout the body. Blood contains, among other things, red blood cells, white blood cells, platelets, proteins, albumins, lipids, salts, ions, hormones, clotting factors, and antibodies. Blood can be obtained from a subject by venipuncture or fingerstick. “Plasma” refers to the liquid, cell-free component of blood that contains, among other things, antibodies and clotting factors. “Serum” refers to the liquid, cell-free component of blood that contains, among other things, antibodies, after clotting of the cellular components by fibrinogen and other clotting factors.

The terms “function” and “functional” as used herein refer to a biological, enzymatic, or therapeutic function.

The term “isolated” as used herein refers to material that is substantially or essentially free from components that normally accompany it in its native state. For example, an “isolated cell,” as used herein, includes a cell that has been purified from the milieu or organisms in its naturally occurring state, a cell that has been removed from a subject or from a culture, for example, it is not significantly associated with in vivo or in vitro substances.

The term “purity” of any given substance, compound, or material as used herein refers to the actual abundance of the substance, compound, or material relative to the expected abundance. For example, the substance, compound, or material may be at least 80, 85, 90, 91, 92, 93, 94, 95, 96, 97, 98, 99, or 100% pure, including all decimals in between. Purity may be affected by unwanted impurities, including but not limited to side products, isomers, enantiomers, degradation products, solvent, carrier, vehicle, or contaminants, or any combination thereof. Purity can be measured technologies including but not limited to chromatography, liquid chromatography, gas chromatography, spectroscopy, UV-visible spectrometry, infrared spectrometry, mass spectrometry, nuclear magnetic resonance, gravimetry, or titration, or any combination thereof.

Some embodiments disclosed herein related to selecting a subject or patient in need. In some embodiments, a patient is selected who currently has a viral infection. In some embodiments, a patient is selected who is suspected of having a viral infection. In some embodiments, a patient is selected who has previously had a viral infection. In some embodiments, a patient is selected who is at risk of a viral infection. In some embodiments, a patient is selected who has a recurrence of a viral infection. In some embodiments, a patient is selected who may have any combination of the aforementioned selection criteria. In some embodiments, the viral infection is a coronavirus infection. In some embodiments, the viral infection is a SARS-CoV-2 infection. In some embodiments, the viral infection is a non-SARS-CoV-2 coronavirus infection. In some embodiments, the viral infection is not a coronavirus infection. In some embodiments, the viral infection is an influenza virus infection.

Some embodiments disclosed herein related to selecting a subject or patient in need. In some embodiments, a patient is selected who currently has an immune disorder. In some embodiments, a patient is selected who is suspected of having an immune disorder. In some embodiments, a patient is selected who has previously had an immune disorder. In some embodiments, a patient is selected who is at risk of an immune disorder. In some embodiments, a patient is selected who has a recurrence of an immune disorder. In some embodiments, a patient is selected who may have any combination of the aforementioned selection criteria. In some embodiments, the immune disorder is cancer. In some embodiments, the immune disorder is an autoimmune disorder. In some embodiments, the immune disorder is mixed connective tissue disease (MCTD), systemic lupus erythematosus (SLE), antiphospholipid syndrome, autoimmune hepatitis, primary biliary cholangitis, Crohn's disease, ulcerative colitis, dermatomyositis, polymyositis, Grave's disease, Hashimoto's disease, osteoporosis, rheumatoid arthritis, scleroderma, Sjogren's syndrome, hepatitis B, hepatitis C, HIV, or syphilis, or any combination thereof.

The terms “treat”, “treating”, “treatment”, “therapeutic”, or “therapy” as used herein has its ordinary meaning as understood in light of the specification, and do not necessarily mean total cure or abolition of the disease or condition. The term “treating” or “treatment” as used herein (and as well understood in the art) also means an approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delaying or slowing of disease progression, amelioration or palliation of the disease state, diminishment of the reoccurrence of disease, and remission, whether partial or total and whether detectable or undetectable. “Treating” and “treatment” as used herein also include prophylactic treatment. Treatment methods comprise administering to a subject a therapeutically effective amount of an active agent. The administering step may consist of a single administration or may comprise a series of administrations. The compositions are administered to the subject in an amount and for a duration sufficient to treat the patient. The length of the treatment period depends on a variety of factors, such as the severity of the condition, the age and genetic profile of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a subject already suffering from or developing a disease or condition.

The term “nucleic acid” as used herein refers to deoxyribonucleic acid (DNA) or ribonucleic acid (RNA) and known analogs, derivatives, or mimetics thereof. A nucleic acid can be oligomeric and include oligonucleotides, oligonucleosides, oligonucleotide analogs, oligonucleotide mimetics and chimeric combinations of these. A nucleic acid can be single-stranded, double-stranded, circular, branched, or hairpin and can contain structural elements such as internal or terminal bulges or loops. In some embodiments, a nucleic acid can have a length of at least, or at least about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 nucleobases, or any length within any range bounded by two of the above-mentioned lengths.

The terms “peptide”, “polypeptide”, and “protein” as used herein refers to macromolecules comprised of amino acids linked by peptide bonds. The numerous functions of peptides, polypeptides, and proteins are known in the art, and include but are not limited to enzymes, structure, transport, defense, hormones, or signaling. Peptides, polypeptides, and proteins are often, but not always, produced biologically by a ribosomal complex using a nucleic acid template, although chemical syntheses are also available. By manipulating the nucleic acid template, peptide, polypeptide, and protein mutations such as substitutions, deletions, truncations, additions, duplications, or fusions of more than one peptide, polypeptide, or protein can be performed. These fusions of more than one peptide, polypeptide, or protein can be joined in the same molecule adjacently, or with extra amino acids in between, e.g. linkers, repeats, epitopes, or tags, or any other sequence that is 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths. The term “downstream” on a polypeptide as used herein refers to a sequence being after the C-terminus of a previous sequence. The term “upstream” on a polypeptide as used herein refers to a sequence being before the N-terminus of a subsequent sequence.

As used herein, the term “amino acid” refers to either natural and/or unnatural or synthetic amino acids, including glycine and both the D or L optical isomers, and amino acid analogs and peptidomimetics.

A polypeptide or amino acid sequence “derived from” a designated protein refers to the origin of the polypeptide. Preferably, the polypeptide has an amino acid sequence that is essentially identical to that of a polypeptide encoded in the sequence, or a portion thereof wherein the portion consists of at least 10-20 amino acids, or at least 20-30 amino acids, or at least 30-50 amino acids, or which is immunologically identifiable with a polypeptide encoded in the sequence. This terminology also includes a polypeptide expressed from a designated nucleic acid sequence.

### Analytes of Interest

As used herein, the term “antigen” refers to any biological substance that can be bound by an antibody. An antigen may also be an “immunogen” when it can induce an adaptive or humoral immune response in a subject, during which antibodies that are specific to that antigen are generated. An antigen may be a cell, bacteria, virus, or other pathogen, or a component of the cell, bacteria, virus, or other pathogen. A component includes but is not limited to a nucleic acid, nucleotide, DNA, RNA, carbohydrate, sugar, polysaccharide, lipid, cholesterol, protein, polypeptide, peptide, epitope, glycoprotein, lipoprotein, or any fragment thereof. In some embodiments, the antigen is a nucleic acid component of a virus, such as the genetic material of the virus. In some embodiments, the antigen is a polypeptide component of a virus, such as a coat protein, nucleocapsid protein, envelope protein, or a receptor protein. In some embodiments, the antigen is a tumor antigen and is produced by a cancer cell. In other embodiments, the antigen is an autoantigen produced by the subject and may cause an autoimmune disease.

As used herein, the term “analyte” refers to a substance to be detected that may be present in a test sample. Analytes of interest include, but are not limited to, polypeptides, nucleic acids, carbohydrates, or antibodies, or any antigen disclosed herein or otherwise known in the art.

In some embodiments, an analyte of interest is considered a biomarker. As used herein, the term “biomarker” refers to a biomolecule useful for diagnosing or determining the presence, absence, status, stage, or risk of developing a particular disease or condition. Generally, biomarkers are differentially present in samples taken from at least two groups of subjects that differ in health status and can be present at an elevated or decreased level in samples of a first group as compared to samples of a second group.

As used here, a “sample” or “test sample” can include, but is not limited to, biological material obtained from an organism or from components of an organism. The test sample may be of any biological tissue or fluid, for example. In some embodiments, the test sample can be a clinical sample derived from a patient. The test sample can be any of the biological samples disclosed herein or otherwise known in the art. A test sample can also include recombinant cells, cell components, cells grown in vitro, and cell culture constituents including, for example, conditioned medium resulting from the growth of cells in a cell culture medium.

### Capture Probes

In several embodiments, capture probes are attached to a surface of an optical sensor, such as an optical ring resonator. As used herein, a “probe” or “capture probe” is any molecule that can be used to bind to an analyte of interest to visualize or otherwise quantify a certain property, behavior, change, or function of the analyte of interest. For example, a probe may be used to observe the binding activity of an antibody to an antigen. In some embodiments, the probe is an antibody that is specific for another antibody (i.e. secondary antibody). In some embodiments, the probe is an anti-human immunoglobulin antibody, such as an anti-human IgA, anti-human IgD, anti-human IgE, anti-human IgG, or anti-human IgG antibody. In some embodiments, the probe is an antibody produced by an animal, such as a mammal, human, mouse, rat, rabbit, guinea pig, goat, donkey, horse, llama, alpaca, or shark. In some embodiments, the probe is conjugated with a substance or compound that enables or enhances visualization or quantification, such as a fluorescent compound, luminescent compound, dye, radioactive compound, enzyme, protein, nucleic acid, aptamer, ribozyme, substrate, antibiotic, chelator, conjugation reagent, biotin, or heavy metal, or any combination thereof. In some embodiments, the probe is not conjugated with any other substance or compound, or is label-free.

Without being bound by theory, the resonance wavelengths on the optical sensor are sensitive to the local refractive index. Biomolecular binding events that increase the refractive index at the sensor surface can be observed as an increase in the resonance wavelength of the optical sensor. Accordingly, binding of an analyte of interest to a capture probe attached to a surface of an optical sensor represents a “primary” binding event that can be detected and/or measured in terms of an increase in the resonance wavelength of the optical sensor of various embodiments.

Suitable examples of capture probes include, but are not limited to, nucleic acids (e.g. deoxyribonucleic acids and ribonucleic acids), polypeptides (e.g. proteins and enzymes), antibodies, antigens, and lectins. As will be appreciated by one of ordinary skill in the art, any molecule that can specifically associate with an analyte of interest can be used as a capture probe. In certain embodiments, the analyte of interest and capture probe represent a binding pair, which can include but is not limited to antibody/antigen (e.g., nucleic acid or polypeptide), receptor/ligand, polypeptide/nucleic acid, nucleic acid/nucleic acid, enzyme/substrate, carbohydrate/lectin, or polypeptide/polypeptide. It will also be understood that binding pairs of analytes of interest and capture probes described above can be reversed in several embodiments (e.g. in one embodiment an antibody that specifically binds to an antigen can be the analyte of interest and the antigen can be the capture probe, whereas in another embodiment the antibody can be the capture probe and the antigen can be the analyte of interest).

### Polypeptide Capture Probes

In several embodiments, a capture probe attached to a surface of an optical sensor can comprise a polypeptide, which is inclusive of known polypeptide analogs or types. Examples of polypeptide analogs include molecules that comprise a non-naturally occurring amino acid, side chain modification, backbone modification, N-terminal modification, and/or C-terminal modification known in the art. For example, a polypeptide capture probe can comprise a D-amino acid, a non-naturally occurring L-amino acid, such as L-(1-naphthyl)-alanine, L-(2-naphthyl)-alanine, L-cyclohexylalanine, and/or L-2-aminoisobutyric acid.

In several embodiments, a polypeptide capture probe can comprise an antigen to which an antibody analyte of interest is capable of binding. In various aspects, a capture probe can comprise a polypeptide antigen capable of binding to an antibody of interest that is a known biomarker for a particular disease or condition. It will be appreciated that a capture probe of the systems provided herein can comprise any antigen associated with any disease or condition for which a subject's antibody against the antigen is considered a biomarker. As a non-limiting example, a capture probe can comprise a viral antigen capable of binding to an antibody specific against the viral antigen. Presence of such an antibody, as detected by the systems provided herein, would indicate that the subject has been infected by the virus and mounted a specific immune response to it. In certain embodiments, a capture probe can comprise an auto-antigen associated with an autoimmune disorder or an antigen associated with an allergy, which capture probe is capable of binding to an antibody, such as an auto-antibody, of interest. Presence of such an antibody, as detected by the systems provided herein, would indicate that the subject has or is at risk of having the associated autoimmune disorder or allergy.

### Antibody Capture Probes

In some embodiments, a system for detecting the presence of an analyte of interest includes a capture probe comprising an antibody attached to a surface of an optical sensor. In some embodiments, a capture probe comprising an antibody, referred to herein as an “antibody capture probe”, is capable of specifically binding a polypeptide analyte of interest.

As used herein, the terms “antibody” and “immunoglobulin” are intended to include any polypeptide chain-containing molecular structure with a specific shape that fits to and recognizes an antigen or epitope, where one or more non-covalent binding interactions stabilize the complex between the molecular structure and the epitope. In some embodiments, the antibody can bind to an antigen with a dissociation constant (K_{D}) of lower than 10⁻⁶, 10⁻⁷, 10⁻⁸, 10⁻⁹, 10⁻¹⁰, 10⁻¹¹, 10⁻¹², or 10⁻¹³ M. An antibody is produced by a subject by immune cells in response to the presence of the antigen. The antibody is found in biological fluids including but not limited to blood, plasma, serum, lymph, interstitial fluid, mucus secretions, breast milk, saliva, or tears. Testing for the presence of an antibody in one of these fluids by observing binding of the antibody to a specific antigen suggests that the subject has encountered that antigen or the source organism previously or is currently encountering that antigen or the source organism. A serological test determines the presence of an antibody in the blood, plasma, or serum of a subject.

In mammals, immunoglobulins belong to the classes IgA, IgD, IgE, IgG, and IgM. These classes have different protein structures, behaviors, localizations, specificity, and immune cell receptors. IgA is the major antibody found in mucus membranes and mucus secretions. IgG is composed of two heavy chain and two light chain polypeptides linked by disulfide bonds and is the most prevalent immunoglobulin found in blood. IgM is composed of a pentameric or hexameric arrangement of heavy and light chain polypeptides. IgM is the first immunoglobulin that is produced by the host in response to an antigen, after which the smaller, more effective, and more specific IgG are generated. Therefore, the progression of an infection can be tracked by the relative abundances of IgG and IgM to a certain antigen or organism.

In addition to entire immunoglobulins (or their recombinant counterparts), immunoglobulin fragments or “binding fragments” comprising the epitope binding site (e.g., Fab′, F(ab′)₂, single-chain variable fragment (scFv), diabody, minibody, nanobody, single-domain antibody (sdAb), or other fragments) are useful as antibody moieties. Such antibody fragments may be generated from whole immunoglobulins by ricin, pepsin, papain, or other protease cleavage. Minimal immunoglobulins may be designed utilizing recombinant immunoglobulin techniques. For instance “Fv” immunoglobulins for use in the present invention may be produced by linking a variable light chain region to a variable heavy chain region via a peptide linker (e.g., poly-glycine or another sequence which does not form an alpha helix or beta sheet motif). Nanobodies or single-domain antibodies can also be derived from alternative organisms, such as dromedaries, camels, llamas, alpacas, or sharks. In some embodiments, antibodies can be conjugates, e.g. pegylated antibodies, drug, radioisotope, or toxin conjugates.

The antibodies of several embodiments provided herein may be monospecific, bispecific, trispecific, or of greater multi-specificity. Multi-specific antibodies may be specific for different epitopes of a polypeptide or may be specific for more than one polypeptide. See, e.g., PCT publications WO 93/17715; WO 92/08802; WO91/00360; WO 92/05793; Tutt, et al., J. Immunol. 147:60-69 (1991); U.S. Pat. Nos. 4,474,893; 4,714,681; 4,925,648; 5,573,920; 5,601,819; Kostelny et al., J. Immunol. 148:1547-1553 (1992); each of which is incorporated herein by reference in its entirety.

Similar to a sandwich assay format in which an antigen is first bound by a substrate-immobilized primary capture agent and then recognized by a secondary capture agent, the systems of some embodiments provided herein comprise a capture probe (analogous to a sandwich assay primary capture agent) and an antibody (analogous to a sandwich assay secondary capture agent). It is possible to detect and/or measure binding-induced shifts in the resonance wavelength of individual binding events with the systems of various embodiments, including binding of an antibody to the optical sensor. Without being bound by theory, binding of an antibody to the optical sensor can include a change in local refractive index, thereby inducing a detectable and/or measurable shift in a resonance wavelength on the optical sensor.

In several embodiments, a system for detecting and/or measuring an analyte of interest includes an antibody capable of binding to the analyte of interest or a complex or duplex formed between a capture probe attached to a surface of an optical sensor and the analyte of interest. It will be understood that in several embodiments the antibody capable of binding to a complex or duplex formed between a capture probe and analyte of interest can bind to a portion of the analyte of interest that is not bound to the capture probe in formation of the complex or duplex such that the antibody does not directly bind and/or physically contact the capture probe. Thus, the binding of a capture probe/analyte complex by the antibody can be accomplished by the antibody contacting and binding only the analyte portion of the capture probe/analyte complex. In various aspects, an antibody can bind to an epitope on an analyte of interest distinct from the epitope or binding site on the analyte of interest involved in binding to the capture probe. In some aspects, the antibody capable of binding to a complex or duplex formed between a capture probe and analyte of interest binds to the analyte of interest without inhibiting or interfering with the binding between the analyte of interest and the capture probe.

An example of a binding event that increases the refractive index at the optical sensor surface and can be observed as an increase in the resonance wavelength of the optical sensor is an antibody-analyte complex binding to a capture probe attached to a surface of an optical sensor (a “primary” binding event). Yet another detectable and/or measurable binding event is an antibody binding to an analyte of interest which is already bound to a capture probe attached to a surface of an optical sensor (a “secondary” binding event). A further detectable and/or measurable binding event is an antibody binding to a duplex or complex formed between an analyte of interest and a capture probe attached to a surface of an optical sensor (a “secondary” binding event).

It will be understood by a person of ordinary skill in the art that in several aspects, an antibody can bind to the analyte of interest either prior to or after binding between the analyte of interest and capture probe. Thus, in some embodiments a binding-induced shift in the resonance wavelength can be detected and/or measured for (1) an antibody-analyte complex binding to a capture probe attached to a surface on an optical sensor, (2) an antibody binding to the analyte already bound to the capture probe attached to a surface on an optical sensor, or (3) an antibody binding to the duplex or complex formed between the analyte and capture probe attached to a surface on an optical sensor. It will also be apparent to a person of ordinary skill in the art that in some aspects, an antibody is not capable of binding to the capture probe alone or analyte of interest alone, but is capable of binding to the complex or duplex formed between the capture probe and analyte of interest.

Accordingly, certain embodiments drawn to a system for detecting an analyte of interest includes both (1) a capture probe comprising an antibody attached to a surface of an optical sensor and (2) an antibody capable of binding to the analyte of interest either prior to or after binding between the analyte of interest and capture probe. In additional embodiments, a system for detecting an analyte of interest includes (1) a capture probe comprising a nucleic acid attached to a surface of an optical sensor wherein the capture probe is capable of binding to an analyte of interest, and (2) an antibody that is not capable of binding to the capture probe alone or analyte of interest alone, but is capable of binding to the complex or duplex formed between the capture probe and analyte of interest.

### Immunoassays

In some embodiments, the optically based systems disclosed herein are used to perform immunoassays to detect a particular antigen. In some embodiments, the antigen may be from a viral particle. For example, the viral particle may be a coronavirus, such as SARS-CoV-2 or other coronavirus, or other virus, such as the influenza virus, and the optically based systems are used to detect the presence of the viral particle in a biological sample.

The term “coronavirus” as used herein refers to the family of enveloped, positive-sense, single stranded RNA viruses that infect mammals and birds. In humans, coronavirus infections can cause mild symptoms as a common cold, or more severe respiratory conditions such as severe acute respiratory syndrome (SARS), acute respiratory distress syndrome (ARDS), coughing, congestion, sore throat, shortness of breath, pneumonia, bronchitis, and hypoxia. Other symptoms include but are not limited to fever, fatigue, myalgia, and gastrointestinal symptoms such as vomiting, diarrhea, and abdominal pain. The viral envelope comprises spike (“S”), envelope (“E”), membrane (“M”), and hemagglutinin esterase (“HE”) transmembrane structural proteins. The S protein comprises a receptor binding domain (“RBD”), a highly immunogenic region that determines the host receptor specificity of the virus strain. The viral nucleocapsid comprises multiple nucleocapsid (“N” or “NP”) proteins coating the RNA genome. During infection, the S protein attaches to a host cell receptor and initiate entry into the host cell through endocytosis or fusion of the envelope membrane. The RNA genome is translated by the host ribosome to produce new structural proteins and RNA-dependent RNA polymerases, which replicate the viral genome. Viral particles are assembled in the host endoplasmic reticulum and are shed by Golgi-mediated exocytosis. More information about the structure and infection cycle of coronaviruses can be found in Fehr A R & Perlman S. “Coronaviruses: An Overview of Their Replication and Pathogenesis” Methods Mol. Biol. (2015); 1282:1-23, hereby expressly incorporated by reference in its entirety.

The terms “SARS-CoV-2” and “2019-nCoV” as used herein refers to the coronavirus strain responsible for the human coronavirus disease 2019 (COVID-19) pandemic. The contagiousness, long incubation period, and modern globalization has led to worldwide spread of the virus. Development of SARS and other respiratory issues in infected individuals has resulted in immense stress on medical infrastructure. Treatments and vaccines are only beginning to be approved for SARS-CoV-2 and other coronaviruses in humans. Reference sequences for the SARS-CoV-2 genome are publicly accessible (e.g. NCBI GenBank Accession No. MN908947.3) Like the original SARS virus (SARS-CoV-1), SARS-CoV-2 infects human cells by binding to angiotensin-converting enzyme 2 (ACE2) through the RBD of the S protein. The S protein, RBD domain of the S protein, M protein, E protein, and NP protein are good candidates for the development of detection methods, treatments, prophylaxes, or interventions against SARS-CoV-2 and other coronaviruses. In some embodiments, the SARS-CoV-2 is a SARS-CoV-2 variant. In some embodiments, the SARS-CoV-2 variant is selected from 20I/501Y.V1 (B.1.1.7), 20H/501Y.V2 (B.1.351), 20J/501Y.V3 (P.1), B.1.1.207, VUI-202102/03 (B.1.525), VUI-202101/01 (P.2), VUI-202102/01 (A.23.1), VUI 202102/04 (B.1.1.318), VUI 202103/01 (B.1.324.1), or CAL.20C (B.1.429). The embodiments disclosed herein can also be applied to other coronaviruses, including but not limited to HCoV-229E, HCoV-OC43, SARS-CoV-1, HCoV NL63, HKU1, and MERS-CoV.

Exemplary reference sequences of SARS-CoV-2 proteins or portions thereof are provided herein. SEQ ID NO: 1 refers to the SARS-CoV-2 S protein sequence. SEQ ID NO: 2 refers to the SARS-CoV-2 M protein sequence. SEQ ID NO: 3 refers to the SARS-CoV-2 E protein sequence. SEQ ID NO: 4 refers to the SARS-CoV-2 N protein sequence. Additional SARS-CoV-2 proteins sequences and modifications thereof are also exemplified herein. SEQ ID NO: 5 refers to the RBD of the SARS-CoV-2 S protein, spanning amino acids 319-541 of the S protein (Accession number YP_009724390.1), and further with a C-terminal 10× histidine tag. SEQ ID NO: 6 refers to the S2 subunit of the SARS-CoV-2 S protein, spanning amino acids 686-1213 of the S protein (Accession number YP_009724390.1), and further with a C-terminal 10× histidine tag. SEQ ID NO: 7 refers to the S1 subunit (which contains the RED) of the SARS-CoV-2 protein, spanning amino acids 16-685 of the S protein (Accession number QHD43416.1), and further with a C-terminal glycine/serine linker and 10× histidine tag. SEQ ID NO: 8 refers to the combined S1 and S2 subunits of the SARS-CoV-2 S protein, spanning amino acids 16-1213 of the S protein (Accession number YP_009724390.1), and further with a C-terminal 10× histidine tag. Any of the immunoassays may use one or more of the SARS-CoV-2 proteins and antigens described herein, including those that are otherwise known in the art such as the SARS-CoV-2 RBD, S1 subunit, S2 subunit, S1+S2 subunits, S protein, M protein, E protein, and N protein. For those sequences comprising a histidine tag, linker, or any other exogenous sequence, it is envisioned that a sequence lacking said histidine tag, linker, or other exogenous sequence may be used. Furthermore, in some embodiments, a sequence having at least 85%, 86%, 87%, 88%, 89%, 90%, 91%, 92%, 93%, 94%, 95%, 96%, 97%, 98%, 99%, or 100% homology to any of the sequence disclosed herein, or other sequences of SARS-COV-2 or variants thereof may be used.

The term “influenza” as used herein refers to the influenza or “the flu” disease marked by potentially severe symptoms such as fever, chills, coughing, fatigue, headache, sore throat, and myalgia, or the group of enveloped, negative-sense, single stranded RNA viruses that cause influenza. The group of viruses belong to the family Orthomyxoviridae and include Influenzavirus A, Influenzavirus B, Influenzavirus C, and Influenzavirus C. While all 4 genera cause influenza, Influenzavirus A is the one that largely causes influenza pandemics, such as in humans, with Influenzavirus B being a second major cause. The viruses mutate readily, preventing the development of a vaccine that would protect against all serotypes or strains of influenza virus. A positive influenza infection can be confirmed with techniques known in the art, such as serological testing, immunoassays (e.g. rapid influenza diagnostic test) or reverse transcriptase polymerase chain reaction (RT-PCR).

The term “affinity” refers to the degree to which an antibody binds to an antigen so as to shift the equilibrium of antigen and antibody toward the presence of a complex formed by their binding. Thus, where an antigen and antibody are combined in relatively equal concentration, an antibody of high affinity will bind to the available antigen so as to shift the equilibrium toward high concentration of the resulting complex. An antibody that does not bind to an antigen or binds to the antigen such that under certain conditions, stringent or otherwise, the binding is disrupted, the antibody might be considered to have weak or low affinity. For example, in solutions comprising a detergent such as SDS, Tween-20, Triton X-100, and the like, and/or with blocking proteins such as bovine serum albumin, serum albumin, gelatin, casein, or milk proteins, the binding of an antibody with a weak affinity to an antigen may be disrupted. However, this binding is dependent on various factors, such as the concentration of the detergent and/or blocking proteins, the concentration of the antibody and antigens, and the presence of other components such as salts. It is expected that one skilled in the art can determine the relative affinity of an antibody to an antigen by conventional methods. For the purposes of this disclosure, in some embodiments, an antibody with weak affinity to an antigen may be considered to have a dissociation constant (KD) in the high nanomolar or micromolar, or higher, range. Similarly, in some embodiments, an antibody with high affinity to an antigen may be considered to have a dissociation constant in the low nanomolar or picomolar, or lower, range. However, a determination of relative high or low affinity is dependent on the antigen being tested.

The term “specificity” in a general sense refers to the proportion of actual negatives that are correctly identified as such (i.e. true negative rate). For an immunoassay, “specificity” may refer to the ability of an antibody to bind preferentially to one antigenic site versus a different antigenic site, limiting unwanted cross-reactivity with other antigens. For an immunoassay, “specificity” may also refer to the ability of an immunoassay test for detecting a first antigen (or lack thereof), where the first antigen may be from an cell or virus, or a protein, protein fragment, or polypeptide of a cell or virus, without detecting a second antigen with similarities in structure, function, binding activity, immunogenicity, or sequence to the first antigen of the cell or virus, or a second antigen of another cell or virus belonging to the same serotype, strain, variant, species, genus, family, order, class, phylum, or kingdom with at least 50%, 60%, 70%, 80%, 85%, 90%, 91%, 92%, 93%, 94%, 95%, 96%, 97%, 98%, or 99% similarity in structure, function, binding activity, immunogenicity, or sequence to the first antigen, or any percentage within a range defined by any two of the aforementioned percentages. For example, a specificity value can be determined for an immunoassay testing for a SARS-CoV-2 antigen against another antigen, such as another antigen from the same SARS-CoV-2 virus polypeptide, an antigen from another SARS-CoV-2 virus polypeptide, an antigen from a non-SARS-CoV-2 coronavirus, an antigen from the homologous portion of a non-SARS-CoV-2 coronavirus, or an antigen from a non-SARS-CoV-2 virus, such as the influenza virus. The specificity of an immunoassay test distinguishing a first antigen (test or target antigen) from a second antigen (reference or control antigen), and can be conveyed as a percentage, where a high specificity is at least 75%, 80%, 85%, 90%, 91%, 92%, 93%, 94%, 95%, 96%, 97%, 98%, 99%, or 100%, or a percentage within a range defined by any two of the aforementioned percentages. The % confidence interval, such as a 90%, 95%, or 99% confidence interval, of a specificity value should also be determined, where a high confidence interval is at least 75%, 80%, 85%, 90%, 91%, 92%, 93%, 94%, 95%, 96%, 97%, 98%, 99%, or 100%, or a percentage within a range defined by any two of the aforementioned percentages.

The term “sensitivity” in a general sense refers to the proportion of actual positives that are correctly identified as such (i.e. true positive rate). For an immunoassay, “sensitivity” may refer to the lowest positive detection level of an antigen with an antibody above background or non-specific levels. For an immunoassay, “sensitivity” may also refer to the ability of an immunoassay test to correctly determine the incidence of a certain condition, such as a specific viral infection or immune disorder, for an individual or a population by detecting at least one antigen from a cell or virus, or a protein, protein fragment, or polypeptide of a cell or virus. For example, a sensitivity value can be determined for an immunoassay testing for a SARS-CoV-2 viral infection by detecting the presence or absence at least one antigen from the SARS-CoV-2 virus in biological samples from subjects infected with SARS-CoV-2 and other subjects that do not have a viral infection, or infected with a non-SARS-CoV-2 coronavirus, or another virus such as the influenza virus and determining which subjects indeed had a SARS-CoV-2 infection. A high sensitivity is at least 75%, 80%, 85%, 90%, 91%, 92%, 93%, 94%, 95%, 96%, 97%, 98%, 99%, or 100%, or a percentage within a range defined by any two of the aforementioned percentages. The % confidence interval, such as a 90%, 95%, or 99% confidence interval, of a sensitivity value should also be determined, where a high confidence interval is at least 75%, 80%, 85%, 90%, 91%, 92%, 93%, 94%, 95%, 96%, 97%, 98%, 99%, or 100%, or a percentage within a range defined by any two of the aforementioned percentages.

Determining the specificity and sensitivity of an assay is dependent on various aspects, including the antigen or target detected, which may be one or more antigens or targets, and the type of sample used, the number of replicates performed. The Infectious Diseases Society of America recommends serological SARS-CoV-2 tests to have a high specificity and sensitivity of greater than 99.5% (available on the world wide web at www.idsociety.org/practice-guideline/covid-19-guideline-serology/). In some embodiments of the methods disclosed herein, a correct detection of SARS-CoV-2 antibodies from a sample is able to achieve at least 90%, 91%, 92%, 93%, 94%, 95%, 96%, 97%, 98%, 99%, or 100% specificity, or any specificity within a range defined by any two of the aforementioned specificities. In some embodiments of the methods disclosed herein, a correct detection of SARS-CoV-2 antibodies from a sample is able to achieve at least 90%, 91%, 92%, 93%, 94%, 95%, 96%, 97%, 98%, 99%, or 100% sensitivity, or any sensitivity within a range defined by any two of the aforementioned specificities. It is envisioned that one skilled in the art will be able to determine the specificity and sensitivity of the methods disclosed herein.

The overall efficacy of an immunoassay is determined by its specificity and sensitivity. Typically, a single immunoassay that is specific is less sensitive, and an immunoassay that is sensitive is less specific. Some embodiments herein describe methods of using multiplexed immunoassays to test multiple antigens in parallel to obtain specificity and sensitivity values for individual assays, and combine the values to obtain an overall specificity and sensitivity that is better than any individual assay.

The disclosed assays may also be quantified in other statistics that determine the efficacy and accuracy of the assay, including in comparison to other assays. These quantifications may incorporate aspects, including but not limited to the measured specificity, sensitivity, true positive rate, true negative rate, false positive rate, false negative rate, and the prevalence of the tested disease. For example, the assays may be quantified in terms of its positive predictive value (or precision) and its negative predictive value. The positive predictive value indicates the percentage of tested subjects with a positive test that actually have the disease. The negative predictive value indicates the percentage of tested subjects that do not have the disease. It is envisioned that one skilled in the art is able to determine parameters of an assay, including but not limited to specificity, sensitivity, true positive rate, true negative rate, false positive rate, false negative rate, prevalence of the disease, positive predictive value, and/or negative predictive value based on conventional methods. Therefore, in some embodiments, any of the aforementioned parameters may be determined by detecting changes in resonance wavelength using any of the devices disclosed herein, and determining the presence or absence of certain immunoglobulins in a sample that bind to one or more target antigens.

In some embodiments, the multiplexed immunoassays described herein can be used to serologically test for immunity against the SARS-CoV-2 virus. Several different assays can be prepared with different components of the SARS-CoV-2 virus, such as the S protein, M protein, E protein, and N protein. In some embodiments, a fragment of a protein component can also serve as an antigen. In some embodiments, the fragment of a protein component is chosen based on certain properties, such as homology to other coronavirus proteins or lack thereof, and immunogenicity or lack thereof. In some embodiments, a single chip substrate can comprise multiple selected antigens, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32 antigens. These antigens may, for example, be selected as a combination of antigens with certain properties such that some antigens will result in a specific result for SARS-CoV-2, and other antigens will result in a sensitive result for SARS-CoV-2 or other related coronaviruses. In some embodiments, the immunoassays for all of the antigens are done in parallel using one biological sample. In some embodiments, the multiple immunoassay results result in an overall specificity and sensitivity value for the assay that is superior to any individual immunoassay and provides a higher confidence in determining a possible infection by SARS-CoV-2 or other coronavirus. In some embodiments, antigens from other viruses be tested in parallel. Also envisioned are modifications to the set of antigens tested to accommodate for different purposes, such as the use of mostly high sensitivity antigens for subjects not presenting symptoms of an infection, or mostly high specificity antigens for subjects who may have recovered from a certain infection. In some embodiments, the inclusion of multiple selected antigens, such as those of SARS-CoV-2 or other coronavirus, improves the accuracy and/or precision of the test.

Some embodiments are directed towards detecting the presence or absence of immunoglobulins of the first immunoglobulin type and/or detecting the presence or absence of immunoglobulins of a second immunoglobulin type that are specific for an antigen. Using an optical sensor described herein, the presence or absence of an immunoglobulin can be detected qualitatively or quantitatively. In some embodiments, the quantitative addition or accumulation of mass is detected or measured. In some embodiments, the optical sensor, such as a ring resonator, can be used to quantitatively measure the addition or accumulation of mass of the immunoglobulins as it binds to an antigen that is attached to the optical sensor or other region of the fluidic channel or substrate. This may be considered a direct measurement of the mass of the immunoglobulins specific for an antigen present in the biological sample. In some embodiments, the optical sensor, such as a ring resonator, can be used to quantitatively measure the addition or accumulation of mass of the first probe specific for the first immunoglobulin type, or the mass of the second probe specific for the second immunoglobulin type, or the N^{th} probe specific for the N^{th} immunoglobulin type. This may be considered an indirect measurement of the mass of the immunoglobulins specific for an antigen present in the biological sample.

For immunoassays, the ability for an antibody or immunoglobulin to bind to an antigen is tested, detected, measured, quantified, or observed. The binding affinity of the antibody is determined by the strength of intermolecular forces such as electrostatic interactions, hydrogen bonds, van der Waals forces, and hydrophobic interactions. The qualities of the environment (e.g aqueous solution) in which these interactions occur is important both in living organisms and experimentally. The solutions that contain antibodies and antigens for experimental purposes are generally buffer solutions.

The term “buffer solution” refers to a composition that can effectively maintain the pH value between 6 and 9, with a pKa at 25° C. of about 6 to about 9. The buffer described herein is generally a physiologically compatible buffer that is compatible with the function of enzyme activities and enables biological macromolecules to retain their normal physiological and biochemical functions. Examples of buffers include, but are not limited to, HEPES ((4-(2-hydroxyethyl)-1-piperazineethanesulfonic acid), MOPS (3-(N-morpholino)-propanesulfonic acid), N-tris(hydroxymethyl)methylglycine acid (Tricine), tris(hydroxymethyl)methylamine acid (Tris), piperazine-N,N′-bis(2-ethanesulfonic acid) (PIPES) and acetate or phosphate containing buffers (K2HPO4, KH2PO4, Na2HPO4, NaH2PO4) and the like.

When testing binding activity of an antibody to an antigen, wash buffer solutions are commonly used to remove antibodies that are not specific or are weakly specific to an antigen that is immobilized. After washing, the antibodies that remain behind are ideally ones that bind with high affinity to the antigen and are retained bound to the immobilized antigen. In experimental conditions, the stringency of the wash buffer (as well as any other solution used) can be adjusted using certain solutes or components dissolved in the wash buffer. In some embodiments, the stringency is adjusted using chaotropic agents, detergents, or surfactants, including but not limited to urea, thiourea, guanidine, guanidinium chloride, n-butanol, ethanol, lithium perchlorate, lithium acetate, magnesium chloride, phenol, 2-propanol, sodium dodecyl sulfate, or any combination thereof.

Surfactants used herein may be 2,4,7,9-tetramethyl-5-decyne-4,7-diol ethoxylate average Mₙ 670; 2,4,7,9-tetramethyl-5-decyne-4,7-diol, mixture of (±) and meso 98%; Adogen® 464; ALKANOL® 6112; alkyl polyglycoside; anhydrosorbitol ester; Brij® 58; Brij® 93; Brij® C10; Brij® L4 (polyethylene glycol dodecyl ether); Brij® 010; Brij® 020; Brij® 5100; Brij® S10; Brij® S20; carboxylic amides; carboxylic esters; Cetomacrogol 1000; cetostearyl alcohol; cetyl alcohol; Cocamide diethanolamine (“DEA”); Cocamide monoethanolamine (“MEA”); decyl glucoside; decyl polyglucose; disodium cocoamphodiacetate; ethoxylated aliphatic alcohol; ethoxylated derivatives of anhydrosorbitol ester; ethylenediamine tetrakis(ethoxylate-block-propoxylate) tetrol average Mₙ˜7,200; ethylenediamine tetrakis(ethoxylate-block-propoxylate) tetrol average Mₙ˜8,000; ethylenediamine tetrakis(propoxylate-block-ethoxylate) tetrol average Mₙ˜3,600; glycerol monostearate; glycol esters of fatty acids; IGEPAL CA-630; IGEPAL® CA-520; IGEPAL® CA-720; IGEPAL® CO-520; IGEPAL® CO-630; IGEPAL® CO-720; IGEPAL® CO-890; IGEPAL® DM-970; isoceteth-20; lauryl glucoside; maltosides; MERPOL® A; MERPOL® DA; MERPOL® HCS; MERPOL® OJ; MERPOL® SE; MERPOL® SH; monoalkanolamine condensates; monolaurin; mycosubtilin; narrow-range ethoxylate; N-octyl beta-D-thioglucopyranoside; Nonidet P-40; Nonoxynol-9; Nonoxynols; NP-40; octaethylene glycol monododecyl ether; octyl glucoside; oleyl alcohol; polyethylene glycol (“PEG”)-10 sunflower glycerides; pentaethylene glycol monododecyl ether; polidocanol; Poloxamer; Poloxamer 407; poly(ethylene glycol) (12) tridecyl ether mixture of C11 to C14 iso-alkyl ethers with C13 iso-alkyl predominating; poly(ethylene glycol) (18) tridecyl ether mixture of C11 to C14 iso-alkyl ethers with C13 iso-alkyl predominating; poly(ethylene glycol) sorbitan tetraoleate; poly(ethylene glycol) sorbitol hexaoleate; polyethoxylated tallow amine; polyethylene glycol dodecyl ether; polyethylene glycol esters; polyethylene-block-poly(ethylene glycol) average Mₙ˜1,400; polyethylene-block-poly(ethylene glycol) average Mₙ˜575; polyethylene-block-poly(ethylene glycol) average Mₙ˜875; polyethylene-block-poly(ethylene glycol) average Mₙ˜920; polyglycerol polyricinoleate; polyoxyethylene fatty acid amides; polyoxyethylene surfactants; Polysorbate; Polysorbate 20; Polysorbate 80; sorbitan; sorbitan monolaurate (Span 20); sorbitan monopalmitate (Span 40); sorbitan monostearate (Span 60); sorbitan monooleate (Span 80); sorbitan sesquioleate (Span 83); sorbitan trioleate (Span 85); sorbitan isostearate (Span 120); SP Brij® C2 MBAL-SO-(SG); SP Brij® C2 MBAL-SO-(SG); SP Brij® S2 MBAL; SPAN 20; stearyl alcohol; Surfactin; Triton N-101; Triton X-100; Triton X-100; Triton X-114; Triton X-405; Tween® 20; Tween® 40; Tween® 60; Tween® 80; or Tween® 85, or any combination thereof. The surfactant can be found in a 0.001%, 0.01%, 0.05%, 0.1%, 0.2%, 0.3%, 0.4%, 0.5%, 0.6%, 0.7%, 0.8%, 0.9%, 1%, 2%, 3%, 4%, 5%, 10%, 15%, 20%, 25%, 30%, 40%, or 50% w/w, or 0.001%, 0.01%, 0.05%, 0.1%, 0.2%, 0.3%, 0.4%, 0.5%, 0.6%, 0.7%, 0.8%, 0.9%, 1%, 2%, 3%, 4%, 5%, 10%, 15%, 20%, 25%, 30%, 40%, or 50% v/v percentage, or any percentage within a range defined by any two of the aforementioned percentages in a solution.

### Optical Sensing

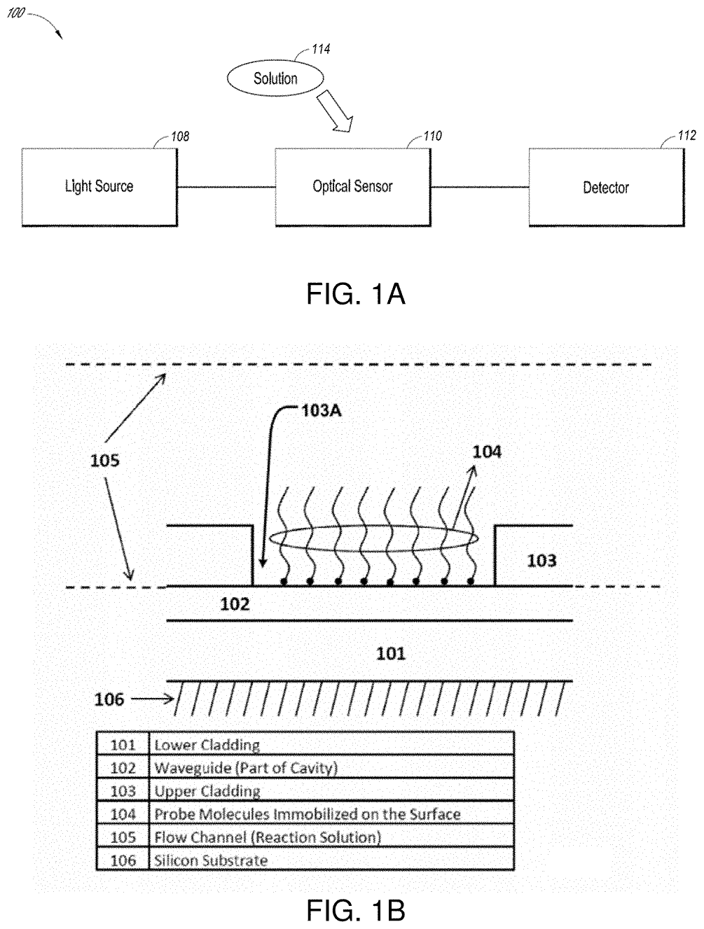

Analyte detection can be accomplished using an optically based system 100 as shown schematically in FIG. 1A. The system 100 includes a light source 108, an optical sensor 110, and an optical detector 112. In various embodiments, the light source 108 outputs a range of wavelengths. For example, the light source 108 may be a relatively narrow-band light source that outputs light having a narrow bandwidth wherein the wavelength of the light source is swept over a region many times the bandwidth of the light source. This light source 108 may, for example, be a laser. This laser may be a tunable laser such that the wavelength of the laser output is varied. In some embodiments, the laser is a diode laser having an external cavity. This laser need not be limited to any particular kind and may, for example, be a fiber laser, a solid state laser, a semiconductor laser or other type of laser or laser system. The laser itself may have a wavelength that is adjustable and that can be scanned or swept. Alternatively, additional optical components can be used to provide different wavelengths. In some embodiments, the light source outputs light having a wavelength for which the waveguide structure is sufficiently optically transmissive. In some embodiments, the waveguide structure is within a sample medium such as an aqueous medium and the light source outputs light having a wavelength for which the medium is substantially optically transmissive such that resonance can be reached in the optical resonator. Additionally, in some embodiments, the light source output has a wavelength in a range where the analyte (e.g., molecules) of interest do not have a non-linear refractive index. Likewise, in various embodiments, the light source 108 may be a coherent light source and output light having a relatively long coherence length. However, in various embodiments, the light source 108 may be a coherent light source that outputs light having a short coherence length. For example, in certain embodiments, a broadband light source such as a super-luminescent light emitting diode (SLED) may be used. In such cases, the wavelength need not be swept. An erbium amplifier running broadband that produces light having a range of wavelengths all at once may also be used. Light from the broadband source extending over an extended spectral range may be injected into the waveguide input. A spectral analyzer (e.g., comprising a spectrometer) may be employed to collect light from the waveguide output and analyze the output spectrum.

The light source 108 provides light to the optical sensor 110. The light source 108 may be controlled by control electronics. These electronics may, for example, control the wavelength of the light source, and in particular, cause the light source 108 to sweep the wavelength of the optical output thereof. In some embodiments, a portion of the light emitted from the light source 108 is sampled to determine, for example, the emission wavelength of the light source.

In some embodiments, the optical sensor 110 comprises a transducer that alters the optical output based on the presence and/or concentration of the analyte to be detected. The optical sensor 110 may include or be a waveguide structure. The optical sensor 110 may be an integrated optical device and may be included on a chip. The optical sensor 110 may comprise semiconductor material such as silicon. The optical sensor 110 may be a resonator structure and/or an interferometric structure (e.g., an interferometer), and may produce an output signal as a result of optical resonance and/or interference. The optical sensor 110 may be included in an array of optical sensors. In some embodiments, the optical sensor 110 is a ring resonator sensor, as described further herein.

The optical detector 112 detects the optical output of the sensor 110. In various embodiments, the optical detector 112 comprises a transducer that converts an optical input into an electrical output. This electrical output may be processed by processing electronics to analyze the output of the sensor 110. The optical detector 112 may comprise a photodiode detector. Other types of detectors 112 may be employed. Collection optics in an optical path between the sensor 110 and the detector 112 may facilitate collection of the optical output of the sensor and direct this output to the detector. Additional optics such as mirrors, beam-splitters, or other components may also be included in the optical path from the sensor 110 to the detector 112.

In various embodiments, the optical sensor 110 is disposed on a chip while the light source 108 and/or the optical detector 112 are separate from the chip. The light source 108 and optical detector 112 may, for example, be part of an apparatus comprising free space optics that interrogates the optical sensors 110 on the chip.

In various embodiments, a solution 114 such as an analyte solution is flowed past the optical sensor 110. The detector 112 detects modulation in an optical signal from the optical sensor 110 when an analyte of interest is detected.

### Optical Ring Resonators

In some embodiments, silicon optical ring resonator sensors are used in a label-free technique to detect molecular activity such as binding of an antibody to an antigen. An optical ring resonator may employ a closed-loop waveguide to propagate light in the form of, for example, whispering gallery modes (WGMs) that result from the total internal reflection of the light along the curved surface of the ring. The WGM may include a surface mode that circulates along the ring resonator surface and interacts repeatedly with any material (e.g. antigen and antibody) on the surface through the WGM evanescent field. Unlike a straight waveguide sensor, the effective light-material interaction length of a ring resonator sensor is no longer determined by the sensor's physical size, but rather by the number of revolutions of the light supported by the resonator, which is characterized by the resonator quality factor, or the Q-factor. The effective length L_{eff} is related to the Q-factor by equation 1 below.

L_{eff}=Qλ/2πn (1)

Where λ is the wavelength of light and n is the refractive index of the ring resonator. Due to the large Q-factor, the ring resonator can provide sensing performance superior to a straight waveguide sensor while using orders of magnitude less surface area and sample volume. In addition, the small size of the ring resonator allows for embodiments with a large number of ring resonators in an array of sensors.

An optical ring resonator on a substrate can be fabricated using, for example, a lithographic technique on relatively cheap silicon-on-insulator (SOI) wafers. Bounding the optical sensor to a substrate can provide a convenient means to handle the optical sensor and to fabricate multiple sensors in arrays. In one example embodiment, 8″ SOI wafers may each contain about 40,000 individually addressable ring resonators. One advantage of using silicon-based technology is that various embodiments may operate in the Si transparency window of around 1.55 μm, a common optical telecommunications wavelength, meaning that lasers and detectors are readily available in the commercial marketplace as plug-and-play components. In other designs, an optical sensor may be detached from a substrate and be free floating.

FIG. 1B illustrates a cross-section of an example optical ring resonator sensor. This sensor includes an optical resonator or an optical interferometric structure that includes a waveguide 102 formed on a substrate 106 which may be, for example, a silicon substrate. A first, lower cladding layer 101 with an index less than that of the waveguide 102 is formed on the substrate 106 and is located beneath the waveguide 102. A second, upper cladding layer 103 is formed over the waveguide 102 and has an index less than that of the waveguide 102. The upper cladding layer 103 is patterned to have one or more regions 103A in which the cladding material for the upper cladding layer 103 is removed to form a sensing region 103A. The sensing region is structured to either completely expose a section of the waveguide 102 or to have a thin layer of the cladding material, to allow a sufficient amount of the optical evanescent field of the guided light in the waveguide 102 to be present in the sensing region 103A. Biological material 104 (e.g., protein, polypeptides, antigens, etc.) is deposited on a surface via a functionalizing process in the sensing region 103A in proximity to the waveguide 102, in such a manner that the evanescent field of the waveguide 102 can interact with the biological material 104. Portions of the upper cladding layer 103 are shown to define one example sensing region 103A that determines which portion of the waveguide 102 is to be functionalized with the biological material 104. A flow channel or fluidic cavity 105 is formed on top of the sensor and a fluidic control mechanism is provided to direct different solutions into the flow channel or fluidic cavity 105 during an antibody-antigen binding process in a sensing region 103A. In addition, the fluidic control mechanism can direct the solutions into the flow channel or fluidic cavity 105 for other molecular processes.

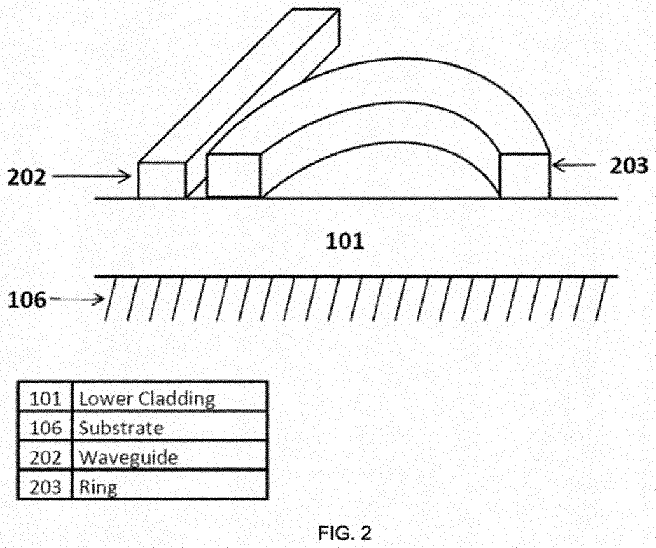

FIG. 2 illustrates a perspective cross section of another example of an optical ring resonator cavity 203 (also referred to as 208 throughout the disclosure and figures herein) and a coupling waveguide 202, formed on a silicon substrate 106. The waveguides 202 and 203 are displaced from the substrate via a buried insulator layer 101 as the lower cladding layer, which may be, for example, silicon dioxide. Functionalization can occur in proximity to the surface(s) of the ring resonator cavity 203. In one implementation, similar to the design in FIG. 1, an upper cladding layer over the ring resonator cavity 203 can be patterned to form sensing regions in proximity to the surface of the ring resonator cavity 203.

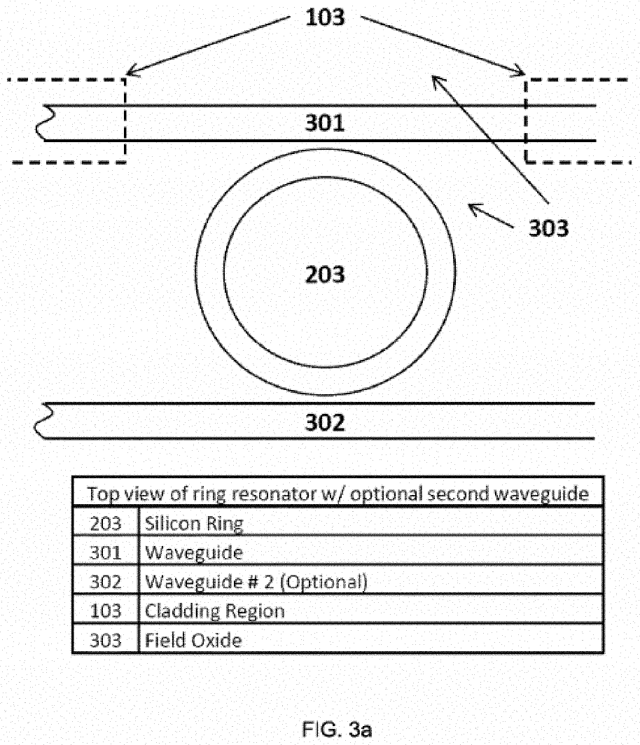

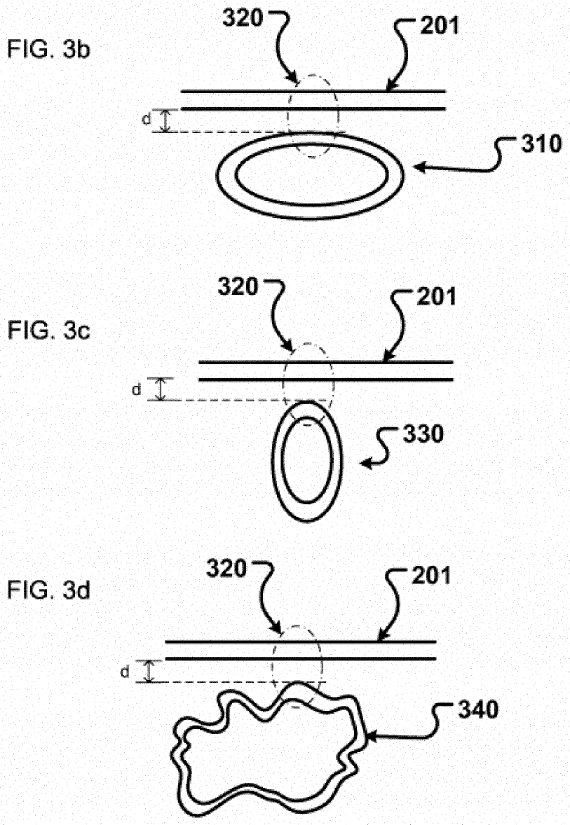

FIG. 3a illustrates a top down view of another example of an optical ring resonator cavity 203 and two coupling waveguides 301 and 302 in evanescent coupling to the ring resonator cavity 203. An upper cladding layer 103 is formed over the first waveguide 301 and is patterned to define one or more sensing regions above the first waveguide 301 and/or the optical ring resonator cavity 203, as shown in FIG. 1B. The cladding layer 103 can be used to confine the interaction of the biological material in each sensing region to be solely to the immediate proximity of the ring 203. The second waveguide 302 is an optical waveguide and may be used to input or output light to or from the optical ring resonator cavity 203.

The ring resonator cavity 203 of FIGS. 2 and 3 can be formed by a waveguide in a closed loop in various configurations. In FIG. 3a, the ring resonator cavity is a closed waveguide loop of a circular shape. This circular closed waveguide loop can support one or more WGMs along the circular path of the closed waveguide loop at and around the outer surface of the circular waveguide and may be independent of the inner surface of the circular waveguide because the WGM exists at and around the outer surface of the circular waveguide. The optical input to the ring resonant cavity 203 can be achieved via evanescent coupling between the waveguide 301 and the ring resonator cavity 203 which are spaced from each other. In other implementations, the closed waveguide loop may be in a non-circular shape that does not support a WGM. FIGS. 3b, 3c and 3d show example shapes of non-circular ring resonator cavities which operate based on the waveguide modes rather than whispering gallery modes. A waveguide mode is supported by the waveguide structure including both the outer and inner surfaces as the waveguide boundaries and thus is different from a WGM. Each ring resonator cavity is spaced from the waveguide 201 by a distance d that is selected to provide desired evanescent coupling. The evanescent coupling configuration is indicated by the numeral 320. One aspect of such a non-circular closed waveguide loop forming the ring resonator cavity is to provide the same evanescent coupling configuration 320 while providing different closed loop waveguides. FIG. 3b and FIG. 3c show a ring resonator cavity in an elliptical shape in a waveguide mode in two different orientations 310 and 330. The specific geometries of the closed waveguide loop can be selected based on the need of a specific sensor design. A race-track shaped closed waveguide loop, for example, may be used. FIG. 3d shows an example where the closed waveguide loop 340 has an irregular shape that can be designed to fit on a chip. A ring resonator cavity may be used to achieve a high Q-factor in part due to re-circulation of the guided optical signal, and such a high Q-factor can be exploited to achieve a high detection sensitivity in detecting a minute amount of a material on the surface of the ring resonator cavity in a label-free molecular process based on optical sensing and monitoring.

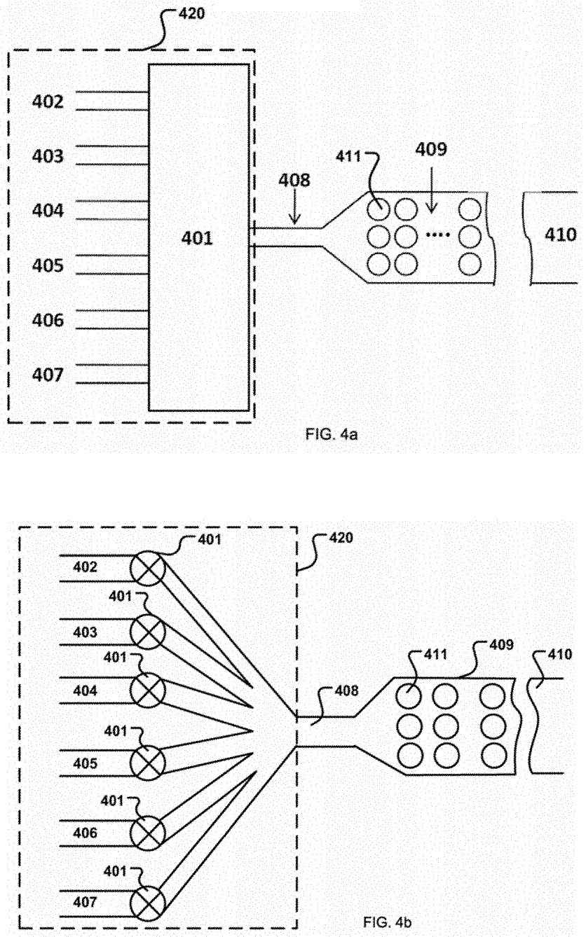

FIG. 4a illustrates a schematic of a monitoring system with a fluid flow control module 420 and an optical sensor array 409 based on label-free optical sensors. The fluid flow control module 420 includes fluid receiving units, such as ports 402, 403, 404, 405, 406 and 407 to receive various fluid types into the fluid flow control module. Also, one or more switches 401 are provided in the fluid flow control module to selectively switch-in or receive one or more of the fluid types into the fluid flow control module. The sensor array 409 includes a matrix of label-free sensors 411 arranged in various configurations. For example, the label-free sensors 411 can be arranged in a square or rectangular configuration with N number of rows and M number of columns of sensors. The label-free sensors 411 can be arranged in other configurations, such as a circle or a triangle. The label-free sensors 411 may be optical ring resonators shown in the examples in FIGS. 1-3b and other sensor designs.

The fluid flow control module 420 is connected to the sensor array 409 using a flow channel 408. Solutions in the fluid flow control module 420 can flow through the flow channel 408 and arrive at the sensor array 409. Different solutions can be obtained in the fluid flow control module 420 by receiving the various fluid types by using the switch 401, and mixing the received fluids. For example, a mix of various biological materials and the associated assay compounds can be added through ports 402-405. In addition, various washing and cleaning solutions, such as buffers can be switched in through ports 406 and 407. The amount and type of fluids to receive and mix in the fluid flow control module 420 can be controlled using the one or more of the switches 401. After the fluids are combined and mixed in a junction region in the fluid flow control module 420, the resultant solution can be applied through the fluid channel 408 and over the sensor array 409.

The solution from the fluid flow control module 420 flows over the sensor array 409 and exits the system through the fluid exit 410. Thus, a continuous flow of solutions can be provided across the sensor array 409. In some implementations, the solution can be held static in the sensor array 409 by stopping the flow.

FIG. 4b shows another example of a monitoring system with a fluid flow control module 420 and a sensor array 409 based on label-free sensors. Each of the fluid input units 402, 403, 404, 405, 406 and 407 is connected to a respective switch 401. To selectively input a fluid type through one of the fluid input units 402, 403, 404, 405, 406 and 407, the respective switch is used. Remaining components of the monitoring system are similar to the system shown in FIG. 4a.

In the label-free sensors of the sensor array 409, the sensor surface can be functionalized to have at least one target molecule (e.g. antigen, antibody, etc.) held within an optical mode, for example by attachment to the sensor surface. Functionalizing the sensor surface can be accomplished by various surface chemistry techniques. Methods of attaching a target molecule to a substrate comprising an optical sensor are described in U.S. Pub. No. 2013/0295688, hereby expressly incorporated by reference in its entirety. In some embodiments, the target molecules are attached to a surface of an optical sensor by a linkage, which may comprise any moiety, functionalization, or modification of the binding surface and/or antigen that facilitates the attachment of the antigen to the surface of the optical sensor. The linkage between the antigen and the surface of the optical sensor can comprise one or more chemical bonds; one or more non-covalent chemical bonds such as Van der Waals forces, hydrogen bonding, electrostatic interaction, hydrophobic interaction, or hydrophilic interaction; and/or chemical linkers that provide such bonds.

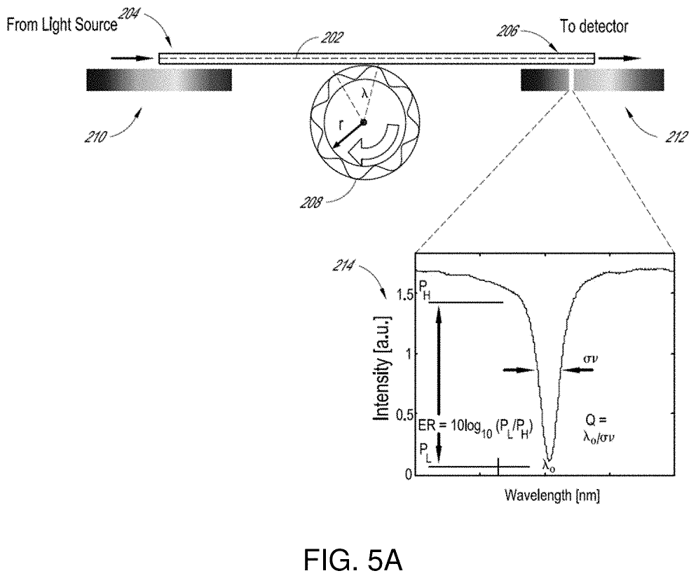

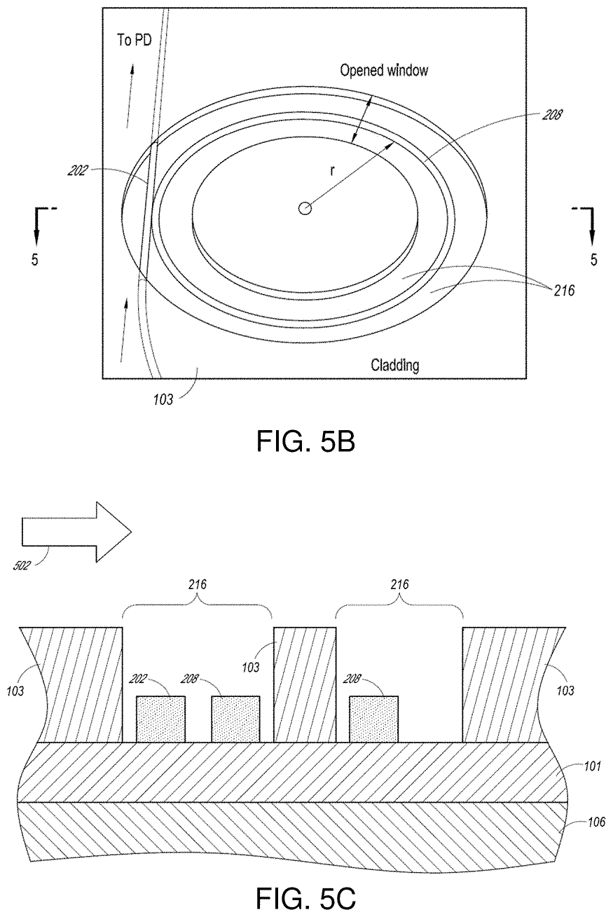

As described herein, ring resonators offer highly sensitive optical sensors that can be prepared so as to detect analytes. The operation of a ring resonator is shown in connection with FIG. 5A. In this configuration, the optical sensor 110 includes a linear input/output waveguide 202 having an input 204 and an output 206, and a ring resonator 208 (also referred to as 203 throughout the disclosure and figures herein) disposed in proximity to a portion of the input/output waveguide 202 that is arranged between the input 204 and the output 206. The close proximity facilitates optical coupling between the input/output waveguide 202 and the ring resonator 208, which is also a waveguide. In this example, the input/output waveguide 202 is linear and the ring resonator 208 is circular such that light propagating in the input/output waveguide 202 from the input 204 to the output 206 is coupled into the ring resonator 208 and circulates therein. Other shapes for the input/output waveguide 202 (for example, curved) and ring resonator 208 (e.g., oval, elliptical, triangular, etc.) are also possible.

FIG. 5A shows an input spectrum 210 to represent that the light injected into the waveguide input 204 includes a range of wavelengths, for example, from a narrow band light source having a narrow band peak that is swept over time (or from a broadband light source such as a super-luminescent diode). Similarly, an output spectrum 212 is shown at the waveguide output 206. A portion of this output spectrum 212 is expanded into a plot of intensity versus wavelength 214 and shows a dip or notch in the spectral distribution at the resonance wavelength, Xo, of the ring resonator 208.

Without subscribing to any particular scientific theory, light “resonates” in the ring resonator when the number of wavelengths around the ring (e.g. circumference) is exactly an integer. In this example, for instance, at particular wavelengths, light circulating in the ring resonator 208 is at an optical resonance when: mλ=2πrn, where m is an integer, λ is the wavelength of light, r is the ring radius, and n is the refractive index. In this resonance condition, light circulating in the ring interferes with light propagating within the linear waveguide 202 such that optical intensity 206 at the waveguide output is reduced. Accordingly, this resonance will be measured as an attenuation in the light intensity transmitted down the linear waveguide 202 past the ring resonator 208 as the wavelength is swept by the light source in a manner such as shown in the plot 214 of FIG. 5A.

Notably, the plot 214 in FIG. 5A shows the dip or notch having a width, cm as measured at full width half maximum (FWHM) and an associated cavity Q or quality factor, Q=λ₀/συ. The ring resonator 208 produces a relatively high cavity Q and associated extinction ratio (ER) that causes the optical sensor 110 to have a heightened sensitivity.

FIG. 5B is a drawing of another example biosensor waveguide structure comprising a linear waveguide 202 and a ring resonator 208. An upper cladding 103 is disposed over most of the area shown. However, a window 216 (here annular in shape) is included in the upper cladding 103 and provides exposure to portions of the linear waveguide 202 and the ring resonator 208. An analyte solution can thereby be flowed across the linear waveguide 202 and ring resonator 208 and permitted to interact therewith. The upper cladding 103 limits the exposure of the integrated waveguide structure to the analyte solution.

FIG. 5C shows a cross-section through the line 5-5 shown in FIG. 5B. The cross-section shows the linear waveguide 202 and the ring resonator 208 disposed over the lower cladding 101 and substrate 106. The upper cladding 103 is also illustrated. As discussed above, openings or windows 216 in the upper cladding 103 provide access for the analyte solution to the linear waveguide 202 and ring resonator 208. A flow channel 502 (shown schematically by an arrow) for the analyte solution is also illustrated.

In some embodiments, an operable sensor chip comprises at least one active optical ring resonator or optical sensor, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32 ring resonators or optical sensors, or any number of ring resonators or optical sensors within a range defined by any two of the aforementioned numbers. The number of ring resonators or optical sensors can be outside these ranges as well. In some embodiments, an operable sensor chip comprises a plurality of active optical ring resonators or optical sensors.

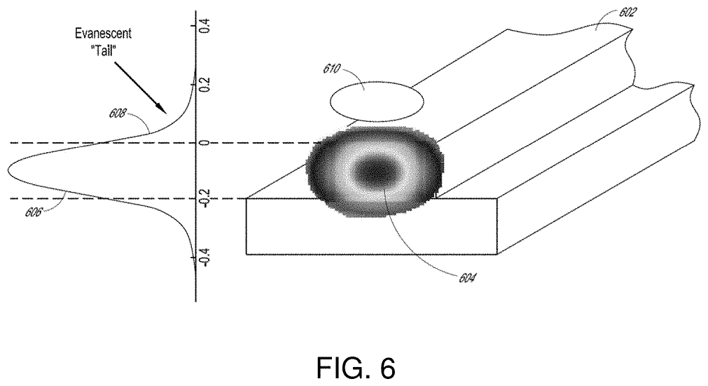

As is well known, light propagates within waveguides via total internal reflection. The waveguide supports modes that yield a spatially varying intensity pattern across the waveguide. As is shown in FIG. 6, a cross-section of a waveguide 602 illustrates an example intensity distribution 604. A plot 606 of the intensity distribution at different heights is provided adjacent to the waveguide structure 602. As illustrated, a portion 608 of the electric field and optical energy referred to as the evanescent “tail” lies outside the bounds of the waveguide 602. The length of this field 608, as measured from the 1/e point, is between 50 and 150 nm, e.g. about 100 nm in some cases. An object 610 located close to the waveguide 602, for example, within this evanescent field length affects the waveguide. In particular, objects 610 within this close proximity to the waveguide 602 affect the effective index of refraction of the waveguide. The effective index of refraction, n, can thus be different when such an object 610 is closely adhered to the waveguide 602 or not. In various embodiments, for example, the presence of an object 610 increases the effective refractive index of the waveguide 602. In this manner, the optical sensor 110 may be perturbed by the presence of an object 610 in the vicinity of the waveguide structure 602 thereby enabling detection. In various embodiments, the size of the object is about the length (e.g. 1/e distance) of the evanescent field to enhance interaction therebetween.

In the case of the ring resonator 208, an increase in the effective refractive index increases the optical path length traveled by light circulating about the ring. Longer wavelengths can resonate in the resonator 208 and, hence, the resonance frequency is shifted to a lower frequency. The shift in the resonant wavelengths of the resonator 208 can therefore be monitored to determine if an object 610 has located itself within close proximity to the optical sensor 110 (e.g., the ring resonator 208 and/or a region of the linear waveguide 202 closest to the ring resonator). A binding event, wherein an object 610 binds to the surface of the optical sensor 110 can thus be detected by obtaining the spectral output 212 from the waveguide output 206 and identifying dips in intensity (or peaks in attenuation) therein and the shift of these dips in intensity.

In various embodiments, the waveguide 602, e.g., the linear waveguide 202 and/or the ring resonator 208 comprise silicon. In some embodiments, the surface of the waveguide 602 may be natively passivated with silicon dioxide. As a result, standard siloxane chemistry may be an effective method for introducing various reactive moieties to the waveguide 602, which are then subsequently used to covalently immobilize biomolecules via a range of standard bioconjugate reactions.

Moreover, the linear waveguide 202, ring resonator 208, and/or additional on-chip optics may be easily fabricated on relatively cheap silicon-on-insulator (SOI) wafers using well established semiconductor fabrication methods, which are extremely scalable, cost effective, and highly reproducible. Additionally, these devices may be easily fabricated and complications due to vibration are reduced when compared to “freestanding” cavities. In one example embodiment, 8″ SOI wafers may each contain about 40,000 individually addressable ring resonators. One advantage of using silicon-based technology is that various embodiments may operate in the Si transparency window of around 1.55 μm, a common optical telecommunications wavelength, meaning that lasers and detectors are readily available in the commercial marketplace as plug-and-play components.

Some embodiments of the waveguides useful with the methods, systems and compositions provided herein include strip and rib waveguides. Other types of waveguides, such as for example, strip-loaded waveguides can also be used. Lower cladding lies beneath the waveguides. In some embodiments, the waveguides are formed from a silicon-on-insulator chip, wherein the silicon is patterned to form the waveguides and the insulator beneath provides the lower cladding. In many of these embodiments, the silicon-on-insulator chip further includes a silicon substrate. Details on the fabrication of silicon biosensor chips can be found in Washburn, A. L., L. C. Gunn, and R. C. Bailey, Analytical Chemistry, 2009, 81(22): p. 9499-9506, and in Bailey, R. C., Washburn, A. L., Qavi, A. J., Iqbal, M., Gleeson, M., Tybor, F., Gunn, L. C. Proceedings of SPIE—The International Society for Optical Engineering, 2009, the disclosures of which are hereby incorporated by reference in their entirety.

Still other designs than those specifically shown in the drawings herein may be employed. More ring resonators may be added. The resonators may also have different sizes and/or shapes. Additionally, the ring resonator(s) may be positioned differently with respect to each other as well as with respect to the input/output waveguide. Likewise, more non-ring resonator waveguides may be added.

In various embodiments, for example, a drop configuration is used. For example, in some such embodiments, a ring resonator is disposed between first and second waveguides. Light (such as a wavelength component) may be directed into an input of the first waveguide and depending on the state of the ring resonator, may be directed to either an output of the first waveguide or an output of the second waveguide. For example, for resonant wavelengths, the light may be output from the second waveguide instead of the first waveguide. An optical detector may thus monitor shifts in intensity peaks to determine the presence of an analyte of interest detected by the optical sensor in some such embodiments.

Combinations of these different features are also possible. Moreover, multiple resonators and/or waveguides may be placed in any desired geometric arrangement. Additionally, spacing between resonators and/or waveguides may be varied as desired. Different features can be combined in different ways.

Also, although linear waveguides are shown as providing access to the ring resonators, these waveguides need not be restricted to plain linear geometry. In some examples, for instance, these waveguides may be curved or otherwise shaped differently. Likewise the ring resonators need not be circularly shaped but can have other shapes. The ring resonators may be oval or elliptically-shaped, triangularly-shaped or irregularly shaped.

Other geometries may possibly be used for the resonator, such as, for example, microsphere, microdisk, and microtoroid structures. See, e.g., Vahala, Nature 2003, 424, 839-846; and in Vollmer & Arnold, Nature Methods 2008, 5, 591-596, the disclosures of which are hereby incorporated by reference in their entirety. Again, combinations of these different features are also possible and different features can be combined in different ways.

Various embodiments of ring resonators and possibly other geometries repeatedly circulate light around, for example, their perimeter, dramatically increasing the optical path length. Furthermore, interference between photons circulating in the structure and those traversing the adjacent waveguide create a resonant cavity of extraordinarily narrow spectral linewidth resulting in a high-Q device. The resulting resonance wavelengths are quite sensitive to changes in the local refractive index. As discussed herein, this sensitivity enables the sensors to detect small masses.

In various embodiments, beads and other particles may be used to provide an amplifying effect on the signal. Other techniques may also be used to provide amplifying effects.

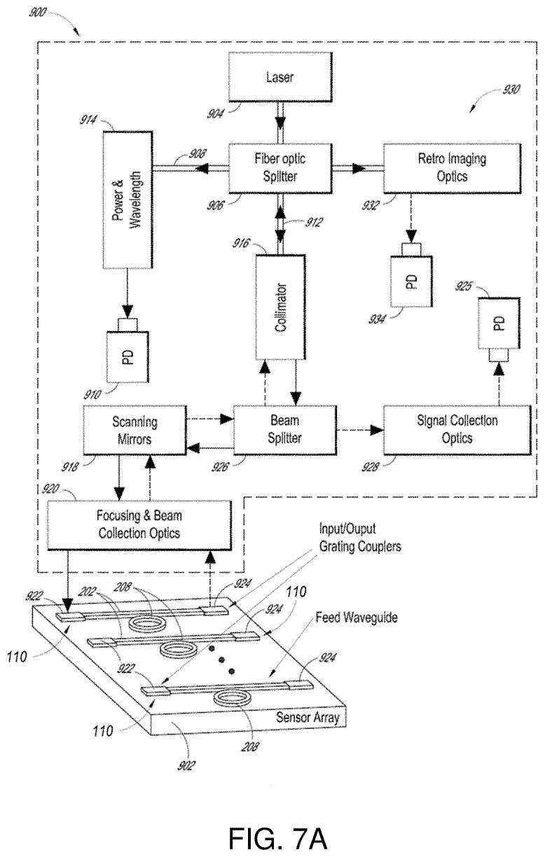

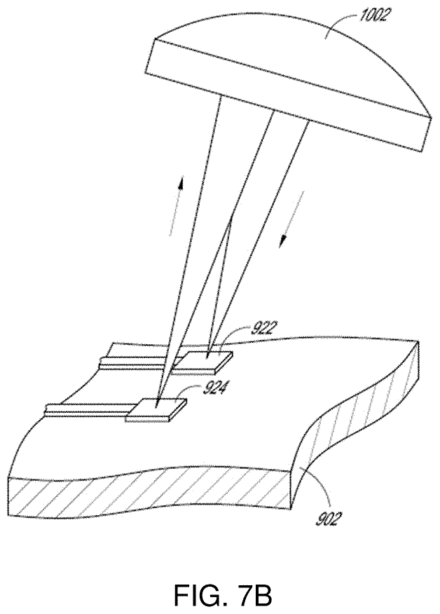

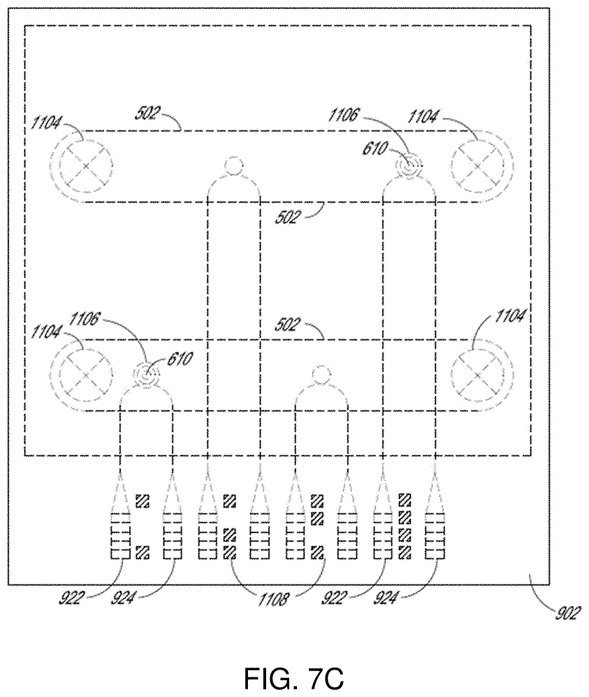

One embodiment of an apparatus 900 for interrogating the optical sensors 110 on a chip 902 is schematically illustrated in FIG. 7A. The apparatus 900 includes a laser light source 904, which may comprise a tunable laser. The apparatus 900 further comprises a splitter 906 that directs light from the laser 904 along a first path 908 to a photodetector 910 for calibration and along a second path 912 toward the chip 902.

A static Fabry-Perot cavity or other wavelength resolving device 914 may be included in the first path 908 to the photodetector 910 such that the photodetector 910 can measure the relative power for different wavelengths of the light output by the laser 904 and presumably provided to the optical sensors 110. The wavelength resolving device 914 may establish a reference wavelength that is known to be output from the light source at a specific time. By additionally knowing the rate at which the wavelengths are swept, the wavelength output by the light source at different times can be determined. Beam shaping optics, such as a collimator 916, may be included in the second optical path 912 to adjust the shape of the beam as desired. This beam is directed to scanning mirrors 918 such that the beam may be scanned across the chip 902. Focusing optics 920 are included to focus the beam onto the chip 902.

The chip 902 includes input couplers 922 configured to couple the beam propagating in free space into the waveguides 202 on the chip. These input couplers 922 may comprise for example waveguide gratings that use diffraction to couple the light beam propagating down toward the chip 902 into optical modes that propagate along the waveguides 922 on the chip. As shown, the chip 902 includes a plurality of optical sensors 110 each comprising linear waveguides 202 and ring resonators 208. The chip 902 additionally includes output couplers 924 that may also comprise waveguide gratings. These grating couplers 924 similarly use diffraction to couple light propagating in optical modes within the waveguides 202 out into free space. Accordingly, light may be injected into the linear waveguides 202 via an input coupler 922 and extracted therefrom via an output coupler 924. As described above, the ring resonators 208 may modulate this light, for example, shifting a wavelength feature such as the spectral valley at the resonance wavelength of the ring resonator, depending on whether an object 610 is in proximity of the resonator.

Light from the output couplers 924 is collected by collection optics. The focusing optics 920 can double as the collection optics. Alternatively, separate collection optics may be used.

The optical detector 112 (comprising a photodetector 925 in FIG. 7A) may be included in the apparatus 900 to detect the light collected from the chip 902. In some embodiments such as illustrated in FIG. 7A, light from the output coupler 924 travels to the photodetector 925 via the collection optics 920, the scanning mirrors 918 as well as a beam-splitter 926 and signal collection optics 928. The scanning mirrors 918 can be scanned so as to direct light collected from different output couplers 924 and hence different optical sensors 110 at different locations on the chip 902.

The apparatus 900 may further comprise an imaging system 930 comprising imaging optics 932 and an image sensor 934. In some embodiments, this image sensor 934 may comprise a single detector that forms an image by recording the detected signal as the scanning mirrors 918 scan the chip. In some embodiments, this image sensor 934 may comprise a detector array such as a CCD or CMOS detector array.

Light from the chip 902 is collected by the collection optics and propagates to the imaging system 930 via the scanning mirrors 918, the beam-splitter 926 (that directs a portion of the light from the output coupler 924 to the detector 106), the collimation optics 916, and the splitter 906 (that also directs light from the laser 904 to the chip). The imaging optics 930 may be used to image the chip 902 and facilitate identification of which optical sensor 110 is being interrogated at a given time. Other configurations are possible.

FIG. 7B shows an example of an objective lens 1002 that operates as the focusing and beam collection optics 920. As illustrated, light is directed into the input coupling element 922 and returned from the output coupling element 924. As illustrated, some embodiments that use grating couplers 922 and 924, which couple free space light into the on-chip optical elements, eliminate the need for any physical connection between the interrogation apparatus 900 and the chip 902.

The system may vary. For example, instead of using a swept light source, such as a tunable laser, a broadband light source such as a super-luminescent diode may be employed.

FIG. 7C schematically illustrates an example chip 902. The chip 902 includes input and output couplers 922, 924, ring resonators 208 and the respective waveguides 202 optically coupled thereto. The chip 902 further includes flow channels 502 configured to direct flow of solution 114 across the optical sensors 110, e.g., the ring resonators 208 and proximal portions of the waveguides 202 optically coupled thereto. Ports 1104 for accessing the flow channels 502 are also included to flow the solution 108 into and out of the flow channels 502.

FIG. 7C shows some optical sensors 1106 of the optical sensors 110 as having an object 610 from the solution 114 coupled to the ring resonators 208. As discussed above, these optical sensors 1106 will have an optical output indicating this event, such as a shift in the spectral feature at the resonance wavelength of the ring resonator 208.

The chip 902 further includes identification markers 1108 for separately identifying the different optical sensors 110. In some example embodiments, identification of the optical sensors 110 is accomplished using the imaging system 930 shown in FIG. 7A, which images and/or collects light from the identification markers 1108. In some embodiments, the identification markers 1108 have unique signatures. Additionally, in some embodiments, the identification markers 1108 are diffractive optical elements. In some embodiments, grating couplers 922 and 924 may be placed in a distinct pattern that allows the unique identification of each optical sensor 110. Accordingly, in such embodiments, separate identification markers 1108 need not be included. Other techniques can also be used for identifying the sensors.

An example apparatus 900 for interrogating the chip 902 having an array of biosensors 110 may include laser 904 comprising a tunable, external cavity diode laser operating with a center wavelength of 1560 nm. A beam from the laser 904 is focused onto a single input grating coupler 922 and rapidly swept through a suitable spectral bandwidth. The light coupled into the input grating coupler 922 is output by the corresponding output grating coupler 924 and is measured. Resonances are measured as wavelengths at which the intensity of light coupled out of the output coupler manifest a notch feature. The different ring resonators 208 in the array may be serially interrogated. However, high tuning rate (e.g., kHz) lasers 904 and fast scan mirrors 918 may allow resonance wavelengths and shifts in wavelength to be determined in near real time with up to 250 ms temporal resolution. In this embodiment, up to 32 optical sensors 110 can be monitored simultaneously during an experiment. Any number of the sensors 110 may be left unexposed to the solution 114 and serve as controls for thermal drift. On-chip and real-time drift compensation can increase sensitivity as temperature dependent refractive index modulations can obscure biomolecular binding events. On-chip referencing is an effective method of compensating for this source of noise. Additional discussion is included in Iqbal, M; Gleeson, M A; Spaugh, B; Tybor, F; Gunn, W G; Hochberg, M; Baehr-Jones, T; Bailey, R C; Gunn, L C, Label-Free Biosensor Arrays based on Silicon Ring Resonators and High-Speed Optical Scanning Instrumentation. IEEE J. Sel. Top. Quantum Electron 2010, 16, 654-66, the disclosure of which is hereby referenced in its entirety.

### Multiplexed Optical Systems

The systems of several embodiments described herein can be used in multiplex formats and/or in real-time. As used herein, “multiplex” can refer to a plurality of different capture probes on the same surface of an optical sensor, or can refer to multiple optical sensors, wherein each sensor can comprise one or more of the same or different capture probes. In the latter sense, multiple optical sensors can be manipulated together temporally or spatially.

In several embodiments, multiple optical sensors can be manipulated in a multiplex format at the same or different times. For example, multiple optical sensors can be manipulated simultaneously or at different times in a multiplex platform, such as a chip, with respect to providing reagent(s) for any of the primary, secondary, or tertiary binding events described herein. In some aspects, a test sample can be provided to multiple optical sensors in a multiplex platform simultaneously. In further aspects, an antibody that specifically binds to an analyte of interest or a duplex/complex formed between an analyte of interest and a capture probe can be provided to multiple optical sensors in a multiplex platform simultaneously. In additional aspects, a particle described herein can be provided to multiple optical sensors in a multiplex platform simultaneously. In certain aspects, a plurality of the same type of particle, such as a universal particle, can be provided to multiple optical sensors in a multiplex platform simultaneously. Multiple optical sensors can also be manipulated simultaneously in a multiplex platform, such as a chip, with respect to detecting or measuring the analyte of interest in parallel. In various embodiments, several optical sensors can be independently monitored in a multiplex format. For example, a plurality of optical rings, wherein each optical ring has a distinct detectable optical property, can be queried or monitored within the same location, such as in a reaction chamber or site on a chip, by a single waveguide.

In some embodiments, reagent(s) for any of the primary, secondary, or tertiary binding events described herein can be administered at different times to populations of optical sensors in a multiplex platform, such as a chip. In other words, a reagent can be provided to one population of optical sensors at a first time, and the reagent can be provided to another population(s) of optical sensors at different time(s), wherein each population comprises one or more optical sensors. In various embodiments, the analyte of interest can be detected in one population of optical sensors at one time and in another population(s) of optical sensors at different time(s), wherein each population comprises one or more optical sensors.

In various embodiments, multiple optical sensors can be spatially manipulated in a multiplex format. In some aspects, reagent(s) for any of the primary, secondary, or tertiary binding events described herein can be differentially administered to distinct populations of optical sensors in a multiplex platform, such as a chip. In other words, a reagent can be provided to one population but not another population of optical sensors in a multiplex platform, wherein each population comprises one or more optical sensors. In various embodiments, the analyte of interest can be detected or measured in one population but not in another population of optical sensors, wherein each population comprises one or more optical sensors.

The multiplex embodiments described above are particularly advantageous in reducing cross-talk from the individual detection systems in a multiplex platform. For instance, by temporally or spatially manipulating distinct populations of optical sensors in a multiplex platform, the extent of cross-talk from the individual detection systems can be reduced. As used herein, the term “cross-talk” refers to a binding event that provides undesired signal detected or measured at any given optical sensor. Cross-talk includes false positive signals or interfering signals resulting from non-specific interaction or binding of reagents from one detection system and another.

For example, in an immunoassay format in which a detection system comprises an antibody capture probe or secondary antibody that is capable of undesirably cross-reacting with antigens that are not analytes of interest for a given optical sensor, it is possible to reduce cross-talk by temporally or spatially segregating the source of cross-talk.

In several embodiments, cross-talk can be temporally reduced by providing reagent(s) for any of the primary, secondary, or tertiary binding events described herein at different times. For example, multiple test samples can be provided at different times (e.g. staggered or sequentially), such that a cross-reacting antigen present in some test samples but not others cannot result in an undesired signal at a given time. Also, different secondary antibodies can be provided at different times to reduce non-specific binding of a secondary antibody, which is intended for use with one population of optical sensors, to an analyte of interest associated with a different population of optical sensors. In various embodiments, cross-talk can be reduced by detecting or measuring an analyte of interest in different populations of optical sensors at different times.

Alternatively or additionally, cross-talk can be spatially reduced by providing reagent(s) for any of the primary, secondary, or tertiary binding events described herein to distinct populations of optical sensors in a multiplex platform. For instance, samples having cross-reacting antigens or secondary antibodies capable of cross-reacting with an antigen that is not an analyte of interest can be kept separated from distinct populations of optical sensors. In various embodiments, a multiplex platform can include different flow cells or channels for providing reagents to spatially separate populations of optical sensors in order to reduce crosstalk.

The multiplex embodiments described above are particularly suited for real-time analyte detection, especially in embodiments with reduced cross-talk. Such binding events detectable in real-time include, but are not limited to, a “primary” binding event between an analyte of interest (with or without a pre-bound particle) and a capture probe, a “secondary” binding event between an antibody (with or without a pre-bound particle) and the analyte of interest already bound to the capture probe, a “secondary” binding event between an antibody (with or without a pre-bound particle) and a duplex or complex formed between the analyte and capture probe, a “secondary” binding event between a particle and the analyte of interest already bound to the capture probe, and a “tertiary” binding event between a particle and antibody already bound to the optical sensor via a “secondary” binding event.

Additional details regarding the sensors and apparatus for interrogating such sensors disclosed herein are included in U.S. Patent Publication 2011/0045472 entitled “Monitoring Enzymatic Process”; PCT Publication WO 2010/062627 entitled “Biosensors Based on Optical Probing and Sensing”; U.S. Patent Publication 2013/0295688 entitled “Optical Analyte Detection Systems and Methods of Use”; U.S. Patent Publication 2013/0261010 entitled “Optical Analyte Detection Systems with Magnetic Enhancement and Methods of Use”; and U.S. Patent Publication 2014/0273029 entitled “Methods and Compositions for Enhancing Immunoassays.”

Additional information regarding ring resonators and the use thereof for detection of antigens can be found in Mudumba S et al. “Photonic ring resonance is a versatile platform for performing multiplex immunoassays in real time” J. Immunol. Methods (498):34-43, U.S. Pat. Nos. 9,846,126, 9,921,165, 9,983,206, 10365224, and Publications WO 2013/138251 and WO 2019/212993, each of which is hereby incorporated by reference in its entirety.

Additional information about sensor chip design and scanning instrumentation (e.g. Maverick detection platform from Genalyte, Inc.) and their use in the quantitation of a range of biomolecular targets, including proteins, are provided in Washburn, A L et al. Anal. Chem. 2009, 81:9499-9506 and Iqbal, M et al. IEEE J. Sel. Top. Quantum Electron. 2010, 16:654-661, each of which are hereby incorporated by reference in its entirety.

### Methods of Use

Disclosed herein in some embodiments are methods of performing a multiplexed immunoassay for detecting multiple antigens. In some embodiments, the methods comprise (a) obtaining a biological sample comprising immunoglobulins, (b) providing a substrate comprising a fluidic channel, where a plurality of different antigens are attached to the fluidic channel at respectively different loci in the fluidic channel, (c) flowing the biological sample through the fluidic channel under conditions that permit immunoglobulins in the biological sample to bind to an antigen attached to the fluidic channel, (d) flowing a wash buffer through the fluidic channel to remove immunoglobulins that do not bind to an antigen or that bind to an antigen with weak affinity from the fluidic channel, and (e) flowing a first probe specific for a first immunoglobulin type through the fluidic channel under conditions that permit the first probe to bind to first immunoglobulins that are bound to the antigens attached to the fluidic channel. In some embodiments, the methods further comprise detecting a signal indicative of the presence or absence of immunoglobulins of the first immunoglobulin type that are specific for an antigen. In some embodiments, the biological sample is from a subject and the methods further comprise (g) determining, based on the presence or absence of immunoglobulins of the first immunoglobulin type that are specific for an antigen, whether or not the subject has an infection or immune disorder of interest and/or whether or not the subject has a second condition, where the plurality of different antigens are selected to improve the specificity and/or sensitivity for the infection or immune disorder of interest. In some embodiments, the subject is a mammal, such as a cat, dog, mouse, rat, rabbit, non-human primate, monkey, or a human.

Also disclosed herein are methods of performing a multiplexed immunoassay for detecting multiple antigens. In some embodiments, the methods comprise (a) obtaining a biological sample comprising immunoglobulins, (b) providing a substrate comprising a fluidic channel and a plurality of optical ring resonators, where the plurality of optical ring resonators is situated within the fluidic channel, and where the optical ring resonators comprise multiple copies of a single antigen, where a plurality of different antigens are attached to different optical ring resonators, (c) flowing the biological sample through the fluidic channel to contact the biological sample with the plurality of optical ring resonators, under conditions that permit immunoglobulins in the biological sample to bind to an antigen of an optical ring resonator, (d) flowing a wash buffer through the fluidic channel to remove immunoglobulins that do not bind to an antigen or that bind to an antigen with weak affinity from the plurality of optical ring resonators, (e) flowing a first probe specific for a first immunoglobulin type through the fluidic channel under conditions that permit the first probe to bind to first immunoglobulins that are bound to the antigen of one of the optical ring resonators, and (f) detecting changes in resonance wavelength for the optical ring resonators during the flowing steps of at least (c) and (e), and optionally, (d). In some embodiments, the methods further comprise (g) determining, based on the detected changes in resonance wavelength for the optical ring resonators, the presence or absence of immunoglobulins of the first immunoglobulin type that are specific for an antigen. In some embodiments, the biological sample is from a subject and the methods further comprise (h) determining, based on the presence or absence of immunoglobulins of the first immunoglobulin type that are specific for an antigen, whether or not the subject has an infection or immune disorder, where the plurality of different antigens are selected to improve the specificity and/or sensitivity for the infection or immune disorder. In some embodiments, the subject is a mammal, such as a cat, dog, mouse, rat, rabbit, non-human primate, monkey, or a human. In some embodiments, the plurality of different antigens comprises 1, 2, 3, 4, 5, 6, 7, 8, 9, 10, 11, 12, 13, 14, 15, 16, 17, 18, 19, 20, 21, 22, 23, 24, 25, 26, 27, or 28 antigens, or more than 28 antigens. In some embodiments, the plurality of optical ring resonators comprises 2, 3, 4, 5, 6, 7, 8, 9, 10, 11, 12, 13, 14, 15, 16, 17, 18, 19, 20, 21, 22, 23, 24, 25, 26, 27, or 28 optical ring resonators, or more than 28 optical ring resonators. Generally, the plurality of optical ring resonators will comprise the same number or more optical ring resonators as there are antigens in the plurality of antigens. In some embodiments, the first immunoglobulin type is IgG, IgM, IgA, IgD, or IgE, or any combination thereof. In some embodiments, the first immunoglobulin type is IgG or IgM, or both. As generally known in the art, the different levels of IgG and IgM in a biological sample indicates the timing of the associated disease or disorder, where IgM is produced first upon the initial exposure of an antigen, and IgG are produced afterwards. In some embodiments, the determining the presence or absence of immunoglobulins of the first immunoglobulin type that are specific for an antigen comprises quantitatively determining the amount of the first immunoglobulins that are specific for an antigen.

Also disclosed herein are methods of performing a multiplexed immunoassay for detecting multiple antigens. In some embodiments, the methods comprise (a) obtaining a biological sample comprising immunoglobulins, (b) providing a substrate comprising a fluidic channel, where a plurality of different antigens are attached to the fluidic channel, (c) flowing the biological sample through the fluidic channel under conditions that permit the immunoglobulins in the biological sample to bind to an antigen attached to the fluidic channel at respectively different loci in the fluidic channel, (d) flowing a wash buffer through the fluidic channel to remove immunoglobulins that do not bind to an antigen or that bind to an antigen with weak affinity from the loci in the fluidic channel, (e) flowing a first probe specific for a first immunoglobulin type through the fluidic channel under conditions that permit the first probe to bind to first immunoglobulins that are bound to the antigens of the loci in the fluidic channel, and (f) flowing a second probe specific for a second immunoglobulin type through the fluidic channel under conditions that permit the second probe to bind to second immunoglobulins that are bound to the antigens of the loci in the fluidic channel. In some embodiments, the methods further comprise (g) detecting the presence or absence of immunoglobulins of the first immunoglobulin type or second immunoglobulin type that are specific for an antigen. In some embodiments, the biological sample is from a subject and the methods further comprise (h) determining, based on the presence or absence of immunoglobulins of the first immunoglobulin type that are specific for an antigen, whether or not the subject has an infection or immune disorder, where the plurality of different antigens are selected to improve the specificity and/or sensitivity for the infection or immune disorder. In some embodiments, the subject is a mammal, such as a cat, dog, mouse, rat, rabbit, non-human primate, monkey, or a human.

Also disclosed herein are methods of performing a multiplexed immunoassay for detecting multiple antigens. In some embodiments, the methods comprise (a) obtaining a biological sample comprising immunoglobulins, (b) providing a substrate comprising a fluidic channel and a plurality of optical ring resonators, where the plurality of optical ring resonators is situated within the fluidic channel, and where the optical ring resonators comprise multiple copies of a single antigen and where a plurality of different antigens are attached to different optical ring resonators, (c) flowing the biological sample through the fluidic channel to contact the biological sample with the plurality of optical ring resonators, under conditions that permit the immunoglobulins in the biological sample to bind to an antigen of an optical ring resonator, (d) flowing a wash buffer through the fluidic channel to remove immunoglobulins that do not bind to an antigen or that bind to an antigen with weak affinity from the plurality of optical ring resonators, (e) flowing a first probe specific for a first immunoglobulin type through the fluidic channel under conditions that permit the first probe to bind to first immunoglobulins that are bound to the antigen of one of the optical ring resonators, (f) flowing a second probe specific for a second immunoglobulin type through the fluidic channel under conditions that permit the second probe to bind to second immunoglobulins that are bound to the antigen of one of the optical ring resonators, and (g) detecting changes in resonance wavelength for optical ring resonators during the flowing steps of at least (c), (e) and (f), and optionally, (d). In some embodiments, the methods further comprise (h) determining, based on the detected changes in resonance wavelength for the optical ring resonators, the presence or absence of immunoglobulins of the first immunoglobulin type or second immunoglobulin type that are specific for an antigen. In some embodiments, the biological sample is from a subject. In some embodiments, the methods further comprise (i) determining, based on the presence or absence of immunoglobulins of the first immunoglobulin type that are specific for an antigen, whether or not the subject has an infection or immune disorder, where the plurality of different antigens are selected to improve the specificity and/or sensitivity for the infection or immune disorder. In some embodiments, the plurality of optical ring resonators comprises 2, 3, 4, 5, 6, 7, 8, 9, 10, 11, 12, 13, 14, 15, 16, 17, 18, 19, 20, 21, 22, 23, 24, 25, 26, 27, or 28 optical ring resonators, or more than 28 optical ring resonators. In some embodiments, the first immunoglobulin type is IgG, IgM, IgA, IgD, or IgE, or any combination thereof and the second immunoglobulin type is IgM, IgG, IgA, IgD, or IgE, or any combination thereof. In some embodiments, the first immunoglobulin type is IgG, and the second immunoglobulin type is IgM. In some embodiments, the determining of the presence or absence of immunoglobulins of the first immunoglobulin type or second immunoglobulin type that are specific for an antigen comprises quantitatively determining the amount of the first immunoglobulins or second immunoglobulins, respectively, that are specific for an antigen.

Also disclosed herein are methods of performing a multiplexed immunoassay for detecting multiple antigens. In some embodiments, the methods comprise (a) flowing a biological sample comprising immunoglobulins from a subject through a fluidic channel of a substrate under conditions that permit immunoglobulins in the biological sample to bind to an antigen attached to the fluidic channel, where a plurality of different antigens are attached to the fluidic channel at respectively different loci in the fluidic channel, and (b) flowing a first probe specific for a first immunoglobulin type through the fluidic channel under conditions that permit the first probe to bind to first immunoglobulins that are bound to the antigens attached to the fluidic channel. In some embodiments, the methods further comprise flowing a wash buffer through the fluidic channel to remove immunoglobulins that do not bind to an antigen or that bind to an antigen with weak affinity from the fluidic channel after the step of (a) and before the step of (b). In some embodiments, the methods further comprise (c) detecting a signal indicative of the presence or absence of immunoglobulins of the first immunoglobulin type that are specific for an antigen. In some embodiments, the methods further comprise (g) determining, based on the presence or absence of immunoglobulins of the first immunoglobulin type that are specific for an antigen, whether or not the subject has an infection or immune disorder of interest and/or whether or not the subject has a second condition, where the plurality of different antigens are selected to improve the specificity and/or sensitivity for the infection or immune disorder of interest.

Also disclosed herein are methods of performing a multiplexed immunoassay for detecting multiple antigens. In some embodiments, the methods comprise (a) providing a substrate comprising a fluidic channel and a plurality of optical ring resonators, where the plurality of optical ring resonators is situated within the fluidic channel, and where the optical ring resonators comprise multiple copies of a single antigen, where a plurality of different antigens are attached to different optical ring resonators, (b) flowing a biological sample comprising immunoglobulins from a subject through the fluidic channel to contact the biological sample with the plurality of optical ring resonators, under conditions that permit immunoglobulins in the biological sample to bind to an antigen of an optical ring resonator, (c) flowing a first probe specific for a first immunoglobulin type through the fluidic channel under conditions that permit the first probe to bind to first immunoglobulins that are bound to the antigen of one of the optical ring resonators, and (d) detecting changes in resonance wavelength for optical ring resonators during the flowing steps of (b)-(c). In some embodiments, the methods further comprise flowing a wash buffer through the fluidic channel to remove immunoglobulins that do not bind to an antigen or that bind to an antigen with weak affinity from the plurality of optical ring resonators after the step of (b) and before the step of (c). In some embodiments, the methods further comprise detecting changes in resonance wavelength for optical ring resonators during the flowing of the wash buffer. In some embodiments, the methods further comprise (e) determining, based on the detected changes in resonance wavelength for the optical ring resonators, the presence or absence of immunoglobulins of the first immunoglobulin type that are specific for an antigen. In some embodiments, the methods further comprise (f) determining, based on the presence or absence of immunoglobulins of the first immunoglobulin type that are specific for an antigen, whether or not the subject has an infection or immune disorder, where the plurality of different antigens are selected to improve the specificity and/or sensitivity for the infection or immune disorder. In some embodiments, the plurality of optical ring resonators comprises 1, 2, 3, 4, 5, 6, 7, 8, 9, 10, 11, 12, 13, 14, 15, 16, 17, 18, 19, 20, 21, 22, 23, 24, 25, 26, 27, or 28 optical ring resonators, or more than 28 optical ring resonators. In some embodiments, the first immunoglobulin type is IgG, IgM, IgA, IgD, or IgE. In some embodiments, the first immunoglobulin type is IgG or IgM. In some embodiments, the determining the presence or absence of immunoglobulins of the first immunoglobulin type that are specific for an antigen comprises quantitatively determining the amount of the first immunoglobulins that are specific for an antigen.

Also disclosed herein are methods of performing a multiplexed immunoassay for detecting multiple antigens. In some embodiments, the methods comprise (a) flowing a biological sample comprising immunoglobulins from a subject through a fluidic channel of a substrate under conditions that permit the immunoglobulins in the biological sample to bind to an antigen attached to the fluidic channel at respectively different loci in the fluidic channel, where a plurality of different antigens are attached to the fluidic channel at respectively different loci in the fluidic channel, (b) flowing a first probe specific for a first immunoglobulin type through the fluidic channel under conditions that permit the first probe to bind to first immunoglobulins that are bound to the antigens of the loci in the fluidic channel, and (c) flowing a second probe specific for a second immunoglobulin type through the fluidic channel under conditions that permit the second probe to bind to second immunoglobulins that are bound to the antigens of the loci in the fluidic channel. In some embodiments, the methods further comprise flowing a wash buffer through the fluidic channel to remove immunoglobulins that do not bind to an antigen or that bind to an antigen with weak affinity from the loci in the fluidic channel after the step of (a) and before the step of (b), and/or after the step of (b) and before the step of (c). In some embodiments, the methods further comprise (d) detecting the presence or absence of immunoglobulins of the first immunoglobulin type or second immunoglobulin type that are specific for an antigen. In some embodiments, the methods further comprise (e) determining, based on the presence or absence of immunoglobulins of the first immunoglobulin type that are specific for an antigen, whether or not the subject has an infection or immune disorder, where the plurality of different antigens are selected to improve the specificity and/or sensitivity for the infection or immune disorder.

Also disclosed herein are methods of performing a multiplexed immunoassay for detecting multiple antigens. In some embodiments, the methods comprise (a) providing a substrate comprising a fluidic channel and a plurality of optical ring resonators, where the plurality of optical ring resonators is situated within the fluidic channel, and where the optical ring resonators comprise multiple copies of a single antigen and where a plurality of different antigens are attached to different optical ring resonator, (b) flowing a biological sample comprising immunoglobulins from a subject through the fluidic channel to contact the biological sample with the plurality of optical ring resonators, under conditions that permit the immunoglobulins in the biological sample to bind to an antigen of an optical ring resonator, (c) flowing a first probe specific for a first immunoglobulin type through the fluidic channel under conditions that permit the first probe to bind to first immunoglobulins that are bound to the antigen of one of the optical ring resonators, (d) flowing a second probe specific for a second immunoglobulin type through the fluidic channel under conditions that permit the second probe to bind to second immunoglobulins that are bound to the antigen of one of the optical ring resonators, and (e) detecting changes in resonance wavelength for optical ring resonators during the flowing steps of (b)-(d). In some embodiments, the methods further comprise flowing a wash buffer through the fluidic channel to remove immunoglobulins that do not bind to an antigen or that bind to an antigen with weak affinity from the plurality of optical ring resonators after the step of (b) and before the step of (c), and/or after the step of (c) and before the step of (d). In some embodiments, the methods further comprise detecting changes in resonance wavelength for optical ring resonators during the flowing of the wash buffer. In some embodiments, the methods further comprise (f) determining, based on the detected changes in resonance wavelength for the optical ring resonators, the presence or absence of immunoglobulins of the first immunoglobulin type or second immunoglobulin type that are specific for an antigen. In some embodiments, the methods further comprise (g) determining, based on the presence or absence of immunoglobulins of the first immunoglobulin type that are specific for an antigen, whether or not the subject has an infection or immune disorder and/or whether or not the subject has a second condition, where the plurality of different antigens are selected to improve the specificity and/or sensitivity for the infection or immune disorder. In some embodiments, the plurality of optical ring resonators comprises 1, 2, 3, 4, 5, 6, 7, 8, 9, 10, 11, 12, 13, 14, 15, 16, 17, 18, 19, 20, 21, 22, 23, 24, 25, 26, 27, or 28 optical ring resonators, or more than 28 optical ring resonators. In some embodiments, the first immunoglobulin type is IgG, IgM, IgA, IgD, or IgE, or any combination thereof and the second immunoglobulin type is IgM, IgG, IgA, IgD, or IgE, or any combination thereof. In some embodiments, the first immunoglobulin type is IgG, and the second immunoglobulin type is IgM. In some embodiments, the determining the presence or absence of immunoglobulins of the first immunoglobulin type or second immunoglobulin type that are specific for an antigen comprises quantitatively determining the amount of the first immunoglobulins or second immunoglobulins, respectively, that are specific for an antigen.

As applied to any of the methods disclosed herein, the infection or immune disorder is a viral, bacterial, or fungal infection. In some embodiments, the infection or immune disorder is a viral infection. In some embodiments, the viral infection is a coronavirus infection. In some embodiments, the coronavirus infection is a SARS-CoV-2 infection. In some embodiments, the plurality of antigens comprises at least one immunogenic peptide or peptide fragment of a SARS-CoV-2 protein selected from the group consisting of the S protein, M protein, N protein, E protein, and HE protein. In some embodiments, the viral infection is not a coronavirus infection. In some embodiments, the viral infection is an influenza infection. In some embodiments, the biological sample is whole blood, plasma, or serum. In some embodiments, the biological sample is provided in a volume of 1000 μL or less, such as 10, 20, 30, 40, 50, 60, 70, 80, 90, 100, 110, 120, 130, 140, 150, 160, 170, 180, 190, 200, 210, 220, 230, 240, 250, 300, 350, 400, 450, 500, 550, 600, 650, 700, 750, 800, 850, 900, 950, or 1000 μL, or any volume within a range defined by any two aforementioned volumes. In some embodiments, the biological sample is provided in a volume of 250 μL or less, such as 10, 20, 30, 40, 50, 60, 70, 80, 90, 100, 110, 120, 130, 140, 150, 160, 170, 180, 190, 200, 210, 220, 230, 240, or 250 μL, or any volume within a range defined by any two aforementioned volumes. In some embodiments, the method is performed within 60 minutes or less, such as 5, 10, 11, 12, 13, 14, 15, 16, 17, 18, 19, 20, 25, 30, 35, 40, 45, 50, 55, or 60 minutes or any time duration within a range defined by any two aforementioned values.

As applied to any of the methods disclosed herein, the plurality of antigens comprises at least one antigen specific for the infection or immune disorder and at least one antigen specific for a second condition. In some embodiments, the second condition may be a second infection or immune disorder. In some embodiments, the second condition may be a variant of the infection or immune disorder. In some embodiments, the at least one antigen specific for the infection or immune disorder is an antigen specific for SARS-CoV-2. In some embodiments, the at least one antigen specific for the infection or immune disorder is an antigen specific for a SARS-CoV-2 variant. In some embodiments, the SARS-CoV-2 variant is selected from 20I/501Y.V1 (B.1.1.7), 20H/501Y.V2 (B. 1.351), 20J/501Y.V3 (P.1), B.1.1.207, VUI-202102/03 (B. 1.525), VUI-202101/01 (P.2), VUI-202102/01 (A.23.1), VUI 202102/04 (B.1.1.318), VUI 202103/01 (B.1.324.1), or CAL.20C (B.1.429). In some embodiments, the at least one antigen specific for a second condition is specific for a SARS-CoV-2 variant. In some embodiments, the at least one antigen specific for a second condition is an antigen specific for a virus selected from the group consisting of non-SARS-CoV-2 coronavirus, influenza virus, and combinations thereof. In some embodiments, the plurality of antigens comprises at least one antigen with high specificity for an immunoglobulin associated with an infection or immune disorder and at least one antigen with high sensitivity for an immunoglobulin associated with the infection or immune disorder. In some embodiments, the plurality of antigens comprises two or more antigens with high specificity for an immunoglobulin associated with an infection or immune disorder and two or more antigens with high sensitivity for an immunoglobulin associated with the infection or immune disorder. In some embodiments, the plurality of antigens comprises 2, 3, 4, 5, 6, 7, 8, 9, or 10 antigens with high specificity for an immunoglobulin associated with an infection or immune disorder and 2, 3, 4, 5, 6, 7, 8, 9, or 10 antigens with high sensitivity for an immunoglobulin associated with the infection or immune disorder. In some embodiments, the methods further comprise combining the measured amount of antigens with different sensitivities for immunoglobulins associated with an infection or immune disorder and the measured amount of antigens with different specificities for immunoglobulins associated with an infection or immune disorder. In some embodiments, the combined measurements provide an overall sensitivity and specificity for an infection or immune disorder. In some embodiments, the infection or immune disorder is a SARS-CoV-2 infection and the at least one antigen with high specificity comprises at least 1, 2, 3, 4, 5, 6, 7, 8, 9, or 10 antigens with protein sequences unique to SARS-CoV-2, and the at least one antigen with high sensitivity comprises at least 1, 2, 3, 4, 5, 6, 7, 8, 9, or 10 antigens with protein sequences that are highly immunogenic but common in Coronaviridae with at least 50%, 60%, 70%, 80%, 85%, 90%, 91%, 92%, 93%, 94%, 95%, 96%, 97%, 98%, 99%, or 100% homology. In some embodiments, the infection or immune disorder is a coronavirus infection and the at least one antigen with high specificity comprises at least 1, 2, 3, 4, 5, 6, 7, 8, 9, or 10 antigens with protein sequences unique to a non-SARS-CoV-2 coronavirus, and the at least one antigen with high sensitivity comprises at least 1, 2, 3, 4, 5, 6, 7, 8, 9, or 10 antigens with protein sequences that are highly immunogenic but common in Coronaviridae with at least 50%, 60%, 70%, 80%, 85%, 90%, 91%, 92%, 93%, 94%, 95%, 96%, 97%, 98%, 99%, or 100% homology. In some embodiments, the presence of immunoglobulins that are specific for an antigen with high specificity reduces a false positive reading of a SARS-CoV-2 infection. In some embodiments, the presence of immunoglobulins that are specific for an antigen with high sensitivity reduces a false negative reading of a SARS-CoV-2 infection. In some embodiments, the SARS-CoV-2 infection is caused by a SARS-CoV-2 variant. In some embodiments, the SARS-CoV-2 variant is selected from 20I/501Y.V1 (B.1.1.7), 20H/501Y.V2 (B.1.351), 20J/501Y.V3 (P.1), B.1.1.207, VUI-202102/03 (B 0.1.525), VUI-202101/01 (P.2), VUI-202102/01 (A.23.1), VUI 202102/04 (B.1.1.318), VUI 202103/01 (B.1.324.1), or CAL.20C (B.1.429). In some embodiments, the plurality of antigens comprise one or more of SEQ ID NOs: 1-8. In some embodiments, the plurality of antigens comprise one or more of SEQ ID NOs: 4-8.

Also disclosed herein are methods of performing a multiplexed immunoassay. In some embodiments, the methods comprise (a) contacting a biological sample from a subject comprising a plurality of immunoglobulins with a plurality of optical ring resonators under conditions that permit immunoglobulins to bind to a plurality of antigens, where each optical ring resonator of the plurality of optical ring resonators comprises multiple copies of a single antigen, such that the plurality of optical ring resonators comprises a plurality of antigens, (b) contacting one or more probes specific to one or more immunoglobulin types with the immunoglobulins bound to the plurality of antigens on the optical ring resonators under conditions that permit the one or more probes to bind to the immunoglobulins, and (c) detecting changes in resonance wavelength for the plurality of optical ring resonators during the contacting step of step (a), step (b), or during both contacting steps (a) and (b). In some embodiments, a change in resonance wavelength for an individual optical ring resonator of the plurality of optical ring resonators comprising the multiple copies of the single antigen indicates that either (1) an immunoglobulin that specifically binds to the single antigen is present in the plurality of immunoglobulins, or (2) the immunoglobulin that specifically binds to the single antigen comprises an immunoglobulin type to which the one or more probes specifically bind, or (3) both (1) and (2). In some embodiments, detecting changes in resonance wavelength during the contacting step of step (a) indicates that (1) the immunoglobulin that specifically binds to the single antigen is present in the plurality of immunoglobulins. In some embodiments, detecting changes in resonance wavelength during the contacting step of step (b) indicates that (2) the immunoglobulin that specifically binds to the single antigen comprises the immunoglobulin type to which the one or more probes specifically bind. In some embodiments, the plurality of optical ring resonators is situated within a fluidic channel. In some embodiments, the fluidic channel is situated within a substrate or device. In some embodiments, the contacting step of step (a) comprises flowing the biological sample through the fluidic channel to contact the biological sample with the plurality of optical ring resonators and the contacting step of step (b) comprises flowing the one or more probes through the fluidic channel to contact the immunoglobulins bound to the plurality of antigens on the optical ring resonators. In some embodiments, the methods further comprise a washing step between the contacting steps of step (a) and step (b), where immunoglobulins that do not bind to the plurality of antigens or that bind to the plurality of antigens with weak affinity are removed from the plurality of optical ring resonators. In some embodiments, the methods further comprise detecting changes in resonance wavelength for the plurality of optical ring resonators during the washing step, or after the washing step and before step (b), or during both the washing step and after the washing step and before step (b). In some embodiments, the washing step comprises flowing a wash buffer through the fluidic channel to contact the wash buffer with the plurality of immunoglobulins and the plurality of optical ring resonators. In some embodiments, the plurality of optical ring resonators comprises 2, 3, 4, 5, 6, 7, 8, 9, 10, 11, 12, 13, 14, 15, 16, 17, 18, 19, 20, 21, 22, 23, 24, 25, 26, 27, or 28 optical ring resonators, or more than 28 optical ring resonators. In some embodiments, the one or more immunoglobulin types comprises IgG, IgM, IgA, IgD, or IgE, or any combination thereof. In some embodiments, the one or more immunoglobulin types comprises IgG and IgM. In some embodiments, the methods further comprise determining, based on the detected changes in resonance wavelength for the plurality of optical ring resonators, the presence or absence of immunoglobulins of the one or more immunoglobulin types that are specific for the plurality of antigens. In some embodiments, the methods further comprise determining, based on the presence or absence of immunoglobulins of the one or more immunoglobulin types that are specific for the plurality of antigens, whether or not the subject has or previously had an infection or immune disorder. In some embodiments, the plurality of antigens are selected to improve the specificity and/or sensitivity for detecting the infection or immune disorder. In some embodiments, the infection or immune disorder is a viral infection. In some embodiments, the viral infection is a coronavirus infection. In some embodiments, the coronavirus infection is a SARS-CoV-2 infection, and the plurality of antigens comprises at least one immunogenic peptide of a SARS-CoV-2 protein. In some embodiments, the SARS-CoV-2 protein is selected from the group consisting of the S protein, M protein, N protein, E protein, and HE protein. In some embodiments, the SARS-CoV-2 infection is caused by a SARS-CoV-2 variant. In some embodiments, the SARS-CoV-2 variant is selected from 20I/501Y.V1 (B.1.1.7), 20H/501Y.V2 (B.1.351), 20J/501Y.V3 (P.1), B.1.1.207, VUI-202102/03 (B 0.1.525), VUI-202101/01 (P.2), VUI-202102/01 (A.23.1), VUI 202102/04 (B.1.1.318), VUI 202103/01 (B.1.324.1), or CAL.20C (B.1.429). In some embodiments, the viral infection is not a coronavirus infection. In some embodiments, the viral infection is an influenza infection. In some embodiments, the biological sample is whole blood, plasma, or serum. In some embodiments, the biological sample comprises a volume of 1000 μL or less, such as 10, 20, 30, 40, 50, 60, 70, 80, 90, 100, 110, 120, 130, 140, 150, 160, 170, 180, 190, 200, 210, 220, 230, 240, 250, 300, 350, 400, 450, 500, 550, 600, 650, 700, 750, 800, 850, 900, 950, or 1000 μL, or any volume within a range defined by any two aforementioned volumes. In some embodiments, the biological sample comprises a volume of 250 μL or less, such as 10, 20, 30, 40, 50, 60, 70, 80, 90, 100, 110, 120, 130, 140, 150, 160, 170, 180, 190, 200, 210, 220, 230, 240, or 250 μL, or any volume within a range defined by any two aforementioned volumes. In some embodiments, the method is performed within 60 minutes or less, such as 5, 10, 11, 12, 13, 14, 15, 16, 17, 18, 19, 20, 25, 30, 35, 40, 45, 50, 55, or 60 minutes or any time duration within a range defined by any two aforementioned values. In some embodiments, the plurality of antigens comprises at least one antigen with high specificity for an immunoglobulin associated with the infection or immune disorder and at least one antigen with high sensitivity for an immunoglobulin associated with the infection or immune disorder. In some embodiments, the plurality of antigens comprises antigens associated with two or more diseases or disorders. In some embodiments, the two or more diseases or disorders comprises a SARS-CoV-2 infection, a SARS-CoV-2 variant infection, a non-SARS-CoV-2 coronavirus infection, a non-SARS-CoV-2 viral infection, influenza, or an immune disorder, or any combination thereof. In some embodiments, the plurality of antigens comprises at least one antigen with high specificity for an immunoglobulin associated with at least one of the two or more diseases or disorders and at least one antigen with high sensitivity for an immunoglobulin associated with at least one of the two or more diseases or disorders. In some embodiments, the methods further comprise determining, based on the detected changes in resonance wavelength for the plurality of optical ring resonators, an overall sensitivity and specificity for the two or more diseases or disorders. In some embodiments, the presence of immunoglobulins that are specific for an antigen with high specificity of the plurality of antigens reduces a false positive reading of the infection or immune disorder, or at least one of the two or more diseases or disorders. In some embodiments, the presence of immunoglobulins that are specific for an antigen with high sensitivity of the plurality of antigens reduces a false negative reading of the infection or immune disorder, or at least one of the two or more diseases or disorders. In some embodiments, the infection or immune disorder, or at least one of the two or more diseases or disorders comprises a SARS-CoV-2 infection, and the plurality of antigens comprises at least 1, 2, 3, 4, 5, 6, 7, 8, 9, or 10 antigens with protein sequences unique to SARS-CoV-2, and the plurality of antigens further comprise at least 1, 2, 3, 4, 5, 6, 7, 8, 9, or 10 antigens with protein sequences that are common in Coronaviridae with at least 50%, 60%, 70%, 80%, 90%, 91%, 92%, 93%, 94%, 95%, 96%, 97%, 98%, 99%, or 100% homology. In some embodiments, the infection or immune disorder, or at least one of the two or more diseases or disorders comprises a SARS-CoV-2 infection, and the plurality of antigens comprises at least 1, 2, 3, 4, 5, 6, 7, 8, 9, or 10 antigens with protein sequences unique to SARS-CoV-2, and the plurality of antigens further comprise at least 1, 2, 3, 4, 5, 6, 7, 8, 9, or 10 antigens with protein sequences that are associated with a virus that is not SARS-CoV-2. In some embodiments, the plurality of antigens comprise one or more of SEQ ID NOs: 1-8. In some embodiments, the plurality of antigens comprise one or more of SEQ ID NOs: 4-8.

While various embodiments have been described in some detail for purposes of clarity and understanding, one skilled in the art will appreciate that various changes in form and detail can be made without departing from the true scope of the invention.

### EXAMPLES

Some aspects of the embodiments discussed above are disclosed in further detail in the following examples, which are not in any way intended to limit the scope of the present disclosure. Those in the art will appreciate that many other embodiments also fall within the scope of the invention, as it is described herein above and in the claims.

The invention is generally disclosed herein using affirmative language to describe the numerous embodiments. The invention also includes embodiments in which subject matter is excluded, in full or in part, such as substances or materials, method steps and conditions, protocols, or procedures.

### Example 1: Multiplexed SARS-CoV-2 Assay

This example describes an immunoassay for the semi-quantitative or quantitative determination of anti-SARS-CoV-2 antibodies in human serum. The presence of anti-SARS-CoV-2 antibodies, in conjunction with clinical findings and other laboratory tests, can aid in the detection of a SARS-CoV-2 infection even in asymptomatic or mildly symptomatic individuals. It is envisioned that this example can be expanded to other viral infections, such as influenza, the common cold, SARS-CoV-2 variants, non-SARS-CoV-2 coronavirus infections, and combinations of these viral infections.

### Summary and Explanation of the Test

Incidence of a SARS-CoV-2 viral infection can be suspected or confirmed by the detection of anti-SARS-CoV-2 antibodies in circulating serum in infected individuals as a natural process of adaptive immunity. The assay described in this example can be used to detect more than one type of immunoglobulin, such as IgA, IgG, and IgM. Not only does this improve the sensitivity and/or specificity of the assay, but also provides information towards the progression of the infection by providing, for example, quantitative measurements of the immunoglobulins.

Alternative technologies such as immunodiffusion, counterimmunoelectrophoresis, and ELISA can be employed as parallel methods to confirm the presence of anti-SARS-CoV-2 antibodies in a subject.

### Principles of the Technology

A multiplex detection technology based on silicon photonics using ring resonance can be used to measure binding of macromolecules to sensors on a miniature silicon chip substrate. Changes in resonance wavelength are detected as macromolecules such as antibodies bind to their respective antigens that are bound to the substrate.

### Principles of the Procedure

Silicon chips were fabricated as generally described in Washburn et al., Analytical Chemistry, 2009. 81(22): p. 9499-9506 and Bailey, R. C. et al., Proceedings of SPIE—The International Society for Optical Engineering, 2009, which are herein incorporated by reference in their entireties.

The silicon chips have 1, 2, 3, or 4 fluidic channels to allow for the possibility to run multiple independent assays. Each channel consists of 1, 2, 3, 4, 5, 6, 7, 8, 9, 10, 11, 12, 13, 14, 15, 16, 17, 18, 19, 20, 21, 22, 23, 24, 25, 26, 27, or 28 ring resonators, or any number of ring resonators within a range defined by any two of the aforementioned numbers, or more than 28 ring resonators. The chip can be assembled into a chip carrier for handling.

The assay chip comprises, consists essentially of, or consists of multiple copies of at least one SARS-CoV-2 antigen, a blank solution, and on-chip controls to evaluate the validity of the assay run. The at least one SARS-CoV-2 antigen is spotted on an available ring resonator. Negative or positive controls are spotted on remaining ring resonators. These negative or positive controls can include but are not limited to human serum albumin (HSA), anti-human IgA, human IgA, anti-human IgG, human IgG, anti-human IgM, human IgM, or antigens from a non-SARS-CoV-2 coronavirus, influenza virus, or other virus.

The biological sample from a subject is added to wells of a stripwell containing a compatible buffer solution. The stripwell and the chip carrier are loaded onto a detection device. When diluted sample (e.g. 1:1, 1:2, 1:4, 1:8, 1:16, 1:32, 1:64, 1:100, 1:200, 1:500, 1:1000, or any dilution within a range defined by any two of the aforementioned ratios, such as 1:51) is flowed over the chip, anti-SARS-CoV-2 antibodies, if present in the sample, will bind to the captured SARS-CoV-2 antigens. Any non-specifically bound antibodies from the sample are removed in a wash step followed by flowing a detection reagent containing anti-human IgG for specific detection of IgG antibodies bound to the immobilized antigen. Optionally, a second detection reagent containing anti-human IgA or IgM can be subsequently flowed with only a brief wash step in between to detect IgA or IgM antibodies bound to the immobilized antigen, respectively. Removal of the previous antibodies is not necessary.

This immunoassay does not require conjugation with a label for the secondary antibody. As more mass is bound during the assay run, the shift in resonant wavelength is measured as a raw result. This raw result is then converted to reportable units (AU/mL) using an analysis algorithm. A lot specific calibration curve is generated and using determined lot coefficients, the raw result is converted to AU/mL.

### Reagents

1) The silicon chip substrate spotted with SARS-CoV-2 antigens and quality control proteins house in a carrier in foil pouch with desiccant.

2) Sealed and prefilled stripwell in foil pouch containing the following reagents: (a) Assay buffer: a clear liquid containing PBS, Tween-20, blocking proteins, and preservative and (b) Assay detection reagent: a clear liquid containing goat anti-human IgG, PBS, Tween-20, blocking proteins, and preservative.

Blocking proteins to reduce non-specific binding of antibodies to surfaces or antigens are known in the art, and include but are not limited to milk proteins, casein, albumin, bovine serum albumin, whole serum, goat serum, or human serum.

### Storage Conditions

Store kits at 2-8° C. Do not freeze. Reagents are stable until the expiration date when stored and handled as directed.

### Specimen Collection

This procedure may be performed with human serum. Alternatively, this procedure may be performed with human plasma or whole blood. Alternatively, this procedure may be performed with other biological samples containing antibodies, such as mucus secretions and breast milk. This procedure can also be performed with serum from an animal other than human.

Microbially contaminated specimens, heat-treated specimens, or specimens containing visible particulates are not typically used. Lipemic or icteric specimens are not typically used. It is recommended to follow the Clinical and Laboratory Standards Institute (CLSI) Document H18-A4 for specimen collection.

### Sample Stability

Serum samples and other biological samples stored at 2-8° C. are typically tested within one week.

### Procedure

Materials provided: 1 stripwell, and 1 silicon microchip housed in carrier.

Additional materials required but not provided: detection device, pipettes able to deliver 5-10 μL, and external positive and negative controls.

Preparation of Patient Specimens

1) Remove the stripwell from foil pouch.

2) Use a sterile P200 pipette tip or equivalent to cut open the upper-left well. Discard tip.

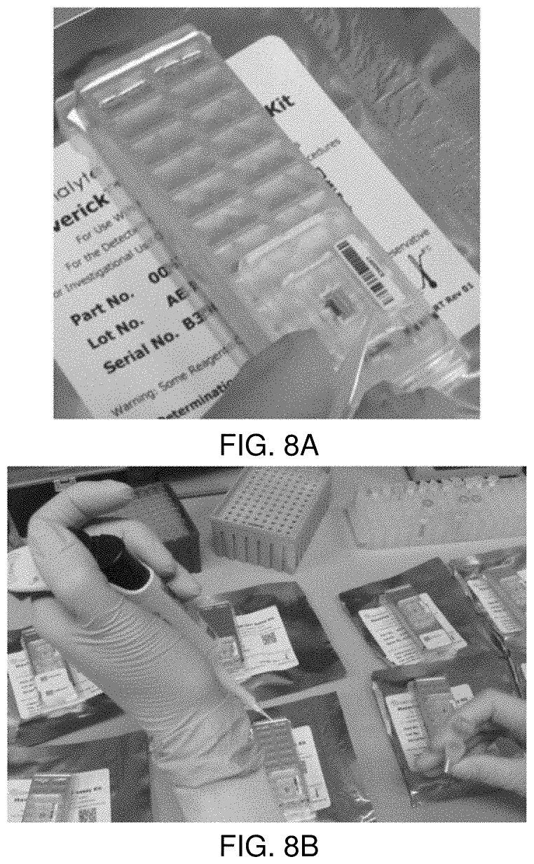

3) Use a second sterile P200 pipette tip to cut open the upper-right well (FIG. 8A). Discard tip.

4) Add 5 μL of serum sample to the upper-left well. Discard tip.

5) Add 5 μL of serum (either same or a different sample) to the upper-right well (FIG. 8B). Discard tip.

6) Use a 200 μL or equivalent pipette set to 50 μL to gently draw and expel ten times to mix specimen in the upper-left well. Discard tip.

7) Use a 200 μL or equivalent pipette set to 50 μL to gently draw and expel ten times to mix specimen in the upper-right well. Discard tip.

8) Repeat using different samples (e.g. from other subject) into remaining wells.

Load Stripwell into Detection Device

1) Orient the stripwell with the notched end toward the instrument.

2) Insert the stripwell into the guide slots in the shuttle plate.

3) Push the stripwell back until it click-locks into position.

Load Chip Carrier

1) Carefully remove the chip carrier from stripwell. (Note: avoid contact with the carrier sippers.)

2) Orient the chip carrier with the sipper tips facing down and away from the instrument.

3) Place the chip carrier on the loading lever.

4) Press down the loading level and hold it down while pushing in the chip carrier.

5) When the chip carrier reaches a hard stop position, release the loading lever. It should return to the horizontal position.

6) Close bay door so it latches.

7) Click Start Test.

Chip Registration

A calibration curve is generated for each lot of the assay kit. When a barcode attached to the kit is read by the detection device, the coefficients for result calculation for that lot is accessed.

The device scans the chip in the carrier and locates all sensors on the chip. Each sensor site is then monitored for changes as the test progresses.

Priming and Test Completion

1) When the countdown timer reaches zero, the screen remains inactive for 1-2 minutes and the status displays Priming. Please wait for the Test Completed prompt.

2) After priming, the device processes the test results and the status displays Idle. It may still take another 30 seconds or so for the Test Completed prompt to appear.

3) When the Test Completed prompt appears, click OK. Then click New Test.

4) In the test bay information screen, click Door Unlock.

5) Remove the stripwell and discard in biohazard waste.

6) Press down the loading bar and hold down while pulling out the chip carrier. Discard the chip carrier in biohazard waste.

### Quality Control

Positive and negative SARS-CoV-2 assay external controls are provided from outside sources. It is recommended that users obtain positive and negative controls to run on a regular basis as needed. Users should also consider national/local regulatory requirements.

### Calculation of Results

A lot specific calibration curve is generated for each kit lot. The coefficients of the curve are used by the detection device to convert the raw result to the reportable arbitrary units (AU/mL). The raw result and reportable arbitrary units are directly proportional to the anti-SARS-CoV-2 antibody levels present in the patient specimen. The results are interpreted as positive or negative for a SARS-CoV-2 infection based on the assay's clinical cutoff.

### Interpretation of Results

Each laboratory is advised to verify the manufacturer provided reference range and may establish its own normal range based upon its own controls and patient population according to their own established procedures.

Results of this assay can be used in conjunction with clinical findings and other serological tests.

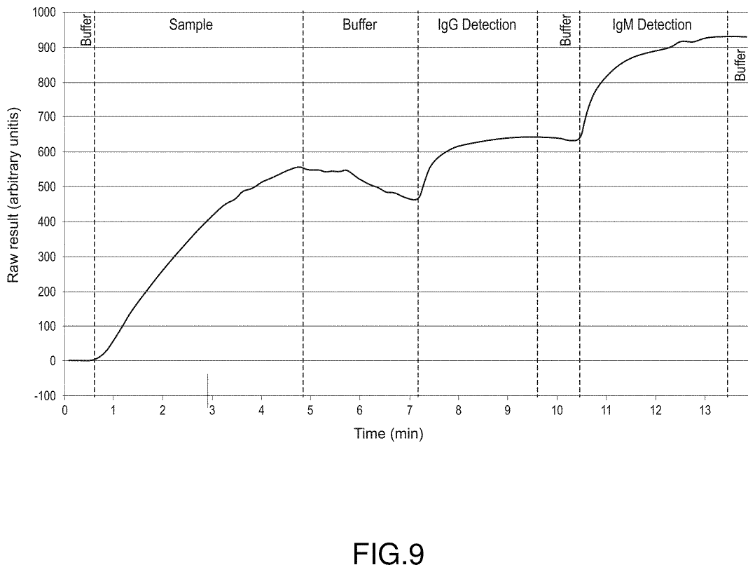

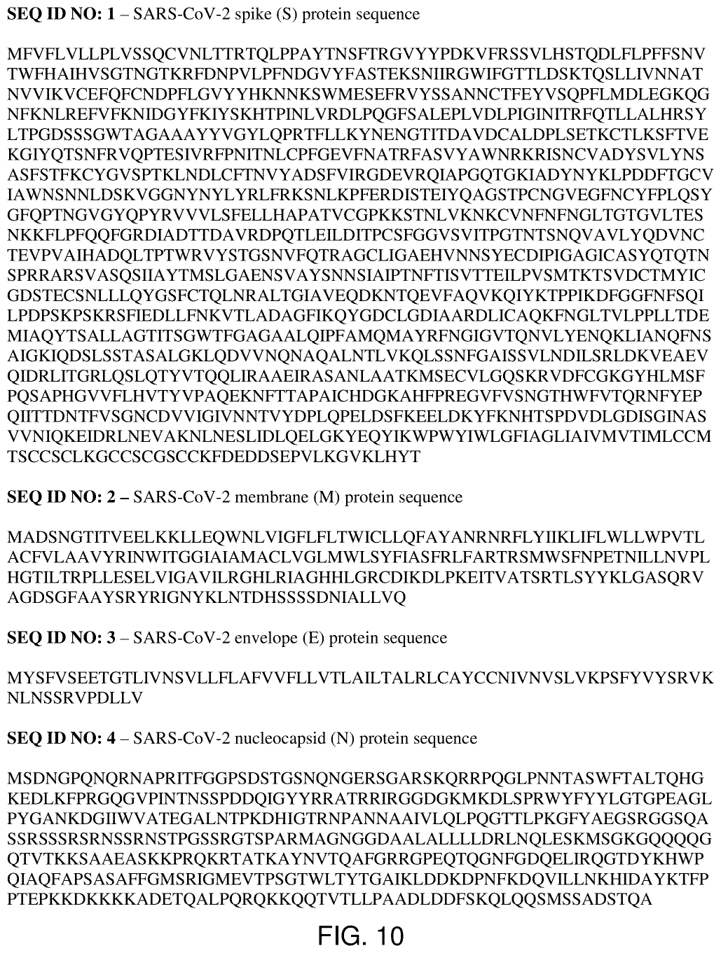

A representative sensogram can be seen in FIG. 9. This sensogram shows the raw result value corresponding to the accumulated binding of anti-SARS-CoV-2 antibodies to a SARS-CoV-2 antigen, and additional binding of anti-human IgG or anti-human IgM to the bound anti-SARS-CoV-2 antibodies. The raw result values can be converted to a reportable arbitrary unit value (AU/mL) to determine a positive or negative result. An example reagent flow sequence is shown in Table 1.

TABLE 1
Reagent flow sequence for FIG. 9

| Reagent | Time (min) | Flow rate (μL/min) |
|---|---|---|
| Buffer | 0.5 | 40 |
| Sample | 4 | 30 |
| Buffer | 2 | 40 |
| IgG detection | 2.5 | 30 |
| Buffer | 1 | 40 |
| IgM detection | 2.5 | 30 |
| Buffer | 1 | 40 |

### Cutoff

The assay cutoff is determined by testing several samples that are known to either have or lack anti-SARS-CoV-2 antibodies. Other samples of unknown status can also be tested to determine or confirm the assay cutoff. The number of samples can be 10, 20, 30, 40, 50, 60, 70, 80, 90, 100, 110, 120, 130, 140, 150, 160, 170, 180, 190, 200, 300, 400, 500, 600, 700, 800, 900, or 1000 samples, or any number of samples within a range defined by any two of the aforementioned numbers. The assay cutoff can be determined to be 0, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 400, or 500 AU/mL, or any value within a range defined by any two of the aforementioned values.

### Expected Values

A panel consisting of at least 10, 20, 30, 40, 50, 60, 70, 80, 90, 100, 110, 120, 130, 140, 150, 160, 170, 180, 190, or 200 presumptively healthy normal volunteers with no history of a SARS-CoV-2 infection or COVID-19 were tested. Biological samples from all individuals report below the assay cutoff.

### Clinical Sensitivity and Specificity

A total of 100, 200, 300, 400, 500, 600, 700, 800, 900 or 100 samples including samples lacking anti-SARS-CoV-2 antibodies and samples containing anti-SARS-CoV-2 antibodies are tested. The clinical sensitivity at a 95% confidence interval for detecting a current or previous SARS-CoV-2 infection is calculated to be at least 75%, 80%, 85%, 86%, 87%, 88%, 89%, 90%, 91%, 92%, 93%, 94%, 95%, 96%, 97%, 98%, 99%, or 100%, or any percentage within a range defined by any two of the aforementioned percentages. The clinical specificity at a 95% confidence interval for detecting a current or previous SARS-CoV-2 infection is calculated to be at least 75%, 80%, 85%, 86%, 87%, 88%, 89%, 90%, 91%, 92%, 93%, 94%, 95%, 96%, 97%, 98%, 99%, or 100%, or any percentage within a range defined by any two of the aforementioned percentages.

### Precision and Reproducibility

The precision and reproducibility of the assay described herein are evaluated according to CLSI EP-5A3—Evaluation of Precision Performance of Quantitative Measurement Procedures.

Precision and repeatability are evaluated by testing 5, 6, 7, 8, 9, or 10 samples with levels covering the assay measuring range. Samples were tested in duplicates per run, two runs per day, per detection device, for 20 days. The precision and repeatability of the assay is determined to have a coefficient of variation of below 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25%.

Kit lot-to-lot reproducibility is evaluated by testing 2, 3, 4, 5, 6, 7, 8, 9, or 10 samples with levels covering the assay measuring range. Samples were tested in replicates of two per run, four runs per day, per detection device, for 5 days using three different kit lots. The kit lot-to-lot reproducibility is determined to have a coefficient of variation of below 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25%.

Device-to-device reproducibility is evaluated by testing 2, 3, 4, 5, 6, 7, 8, 9, or 10 samples with levels covering the assay measuring range. Samples were tested in replicates of two per run, four runs per day, per detection device, for 5 days using three different devices. The kit lot-to-lot reproducibility is determined to have a coefficient of variation of below 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25%.

### Analytical Measuring Range

The Limit of Blank (LOB), Limit of Detection (LOD), and Limit of Quantitation (LOQ) are determined following CLSI EP17-A2—Evaluation of Detection Capability for Clinical Lab Measurement Procedures; Approved Guideline—Second Edition. Four analyte-free serum samples were tested on two kit lots to determine the LOB. LOD was established with 4 low level samples with testing on two kit lots. LOQ was determined by testing 4 low samples on two kit lots in replicates of 12 per sample per kit lot, for 3 days.

The assay's measuring range was evaluated following CLSI guideline EP06-A—Evaluation of Linearity of Quantitative Measurement Procedures: A Statistical Approach. Six serum samples at different levels were serially diluted with analyte-free serum and tested.

The LOB and LOD are determined to have a limit below 0, 1, 2, 3, 4, 5, 6, 7, 8, 9, 10, 20, 30, 40, 50, 60, 70, 80, 90, or 100 AU/mL. The LOQ is determined to have a limit below 0, 1, 2, 3, 4, 5, 6, 7, 8, 9, 10, 20, 30, 40, 50, 60, 70, 80, 90, or 100 AU/mL. The measuring range is determined to have a lower limit below 0, 1, 2, 3, 4, 5, 6, 7, 8, 9, 10, 20, 30, 40, 50, 60, 70, 80, 90, or 100 AU/mL and an upper limit above 10, 20, 30, 40, 50, 60, 70, 80, 90, 100, 200, 300, 400, 500, 600, 700, 800, 900, or 1000 AU/mL.

### Analytical Interference

The assay described herein is evaluated for any potential interference from biological and external interferents. Three samples at levels near LOQ within ±20% cutoff and one medium are tested with 10% spiked interferents and without interferents (controls). Interferents include but are not limited to components found in blood, plasma, or serum, albumin, biotin, cholesterol, bilirubin, cyclophosphamide, diltiazem, hemoglobin, chloroquine, hydroxychloroquine, IgA, IgD, IgE, IgG, IgM, mycophenolate mofetil, naproxen, prednisone, triglycerides, antivirals, or remdesivir.

### Example 2: Quantitative Assessment of Immunoglobulin Titers

The use of the ring resonator sensor for the detection of immunoglobulins in a biological sample of a subject enables the quantitative measurement of the amount of antigen-specific immunoglobulins of different classes, either as an absolute value or relative to other immunoglobulins specific for the same antigen, immunoglobulins of different classes, or immunoglobulins from other biological samples or subjects. This quantitative measurement is preferred if a measurement of a subject's antigen-specific immunoglobulin titer over time is desired. An overall increase in IgG titer towards a particular antigen suggests that a successful and effective immunity has been developed against the source pathogen. A decrease in IgM also provides information regarding the timeline of infection.

Quantitative measurement of immunoglobulin titer is also useful when employed in conjunction with other detection assays. For example, SARS-CoV-2 coronavirus, non-SARS-CoV-2 coronavirus, and other viruses such as influenza virus can be detected using nucleic acid testing with RT-PCR. Nucleic acid testing offers direct detection of viral particles and therefore is only effective during the active portion of infection. By utilizing both tests together and tracking the progression of viral infection in the subject, potential false positive and false negative results in either one test can be reduced.

### Example 3: Additional Exemplary Procedures

The use of the Maverick™ Diagnostics System developed by Genalyte Inc. for the serological detection of anti-SARS-CoV-2 antibodies is disclosed in this example.

As provided herein, a multiplex detection technology based on silicon photonics that uses ring resonance to measure binding of macromolecules to sensors on a miniature silicon chip is used. The Maverick Diagnostic System detects changes in resonance wavelength as macromolecules, such as antibodies, bind to their respective antigens that are bound to the chip. The antigens include 5 SARS-CoV-2 proteins, 4 proteins (one each) to the four human benign coronaviruses, two influenza hemagglutinins, MERS, and SARS-Cov-1 coronavirus proteins (Table 2). The patient sample is added to specific well of a reagent plate that contains the appropriate diluents and buffers. The plate and the chip carrier are loaded into the Maverick instrument. The instrument automates the assay and takes the diluted sample from the reagent plate and flows the sample over the silicon chip, allowing any SARS-CoV-2, common coronavirus, influenza hemagglutinin, SARS-CoV-1, and MERS coronavirus antibodies present in the patient sample to bind to the immobilized antigens. Unbound sample is washed away and then, in succession, goat anti-human IgG, wash solution, and goat anti-human IgM are flowed over the chip to detect the specific class of antibodies bound to any antigens on the chip. The signal is the difference between the successive baseline measurements in GRU (Genalyte Response Units). The antigens used may be substituted for any other SARS-CoV-2 antigen disclosed herein or otherwise known in the art.

TABLE 2
Proteins included on SARS-CoV-2 Multi-Antigen Serology Panel

| Target | Antigen |
|---|---|
| SARS-CoV-2 | Nucleocapsid (SEQ ID NO: 4) |
| SARS-CoV-2 | Spike protein - S1 RBD (SEQ ID NO: 5) |
| SARS-CoV-2 | Spike protein - S2 subunit (SEQ ID NO: 6) |
| SARS-CoV-2 | Spike protein - S1 subunit (SEQ ID NO: 7) |
| SARS-CoV-2 | Spike protein - full length (SEQ ID NO: 8) |
| SARS-CoV-229E | Spike protein |
| SARS-CoV-NL63 | Nucleoprotein |
| SARS-CoV-OC43 | Spike protein |
| SARS-CoV-HKU1 | Spike protein |
| Influenza A | Hemagglutinin H1 |
| Influenza A | Hemagglutinin H3 |
| MERS | Spike protein S1 subunit |
| SARS-CoV-1 | Nucleocapsid |

Reagents and Materials:

Silicon chip spotted with viral antigens, and quality control proteins housed in a carrier in foil pouch with desiccant.

Pregen Reagent Plate: sealed, prefilled in a foil sealed plate containing: Running Buffer (wells A1, A2), clear liquid containing PBS, Tween-20, and preservative; Regeneration Buffer (wells B-F, 1&2), clear liquid containing glycine and SDS solution; TE buffer (wells G1, G2), clear liquid Tris-EDTA buffer.

Reagent Plate: sealed, prefilled in a foil sealed plate containing the following reagents: Running Buffer (wells A-D, 1&2), clear liquid containing PBS, Tween-20, and preservative; SARS-CoV-2 Multi-Antigen Serology Panel Detection Buffer-1 (wells E1, E2), clear liquid containing goat anti-human IgG, PBS, Tween-20, and preservative; SARS-CoV-2 Multi-Antigen Serology Panel Detection Buffer-2 (wells F1, F2), clear liquid containing goat anti-human IgM, PBS, Tween-20, and preservative; Regeneration Buffer (wells G1, G2), clear liquid containing glycine and SDS solution; TE buffer (wells H1, H2), clear liquid Tris-EDTA buffer.

Calibrator: Store calibrator at −20° C. or lower. Once thawed, store the vial at 2-8° C. for no longer than 7 days.

External Quality Controls: Store all controls at −20° C. or lower. Once thawed, store the vial at 2-8° C. for no longer than 7 days.

Specimen Collection:

This procedure may be performed using EDTA venous whole blood, plasma or serum specimens. Microbially contaminated, heat-treated, or specimens containing visible particulates are not typically used. Lipemic or icteric specimens are not typically used. Storage conditions for samples are as follows: 1) Specimens are typically tested as soon as possible after collection, (2) Store EDTA anticoagulant venous whole blood at 2-8° C. if not tested immediately. Do not freeze whole blood, (3) Store serum and EDTA plasma at −20° C. if not tested immediately. Avoid multiple freeze/thaw cycles.

Use of the Maverick Diagnostic System

Before you start, allow all samples and reagents to come to room temperature (20-26° C.) prior to use.

Conditioning Protocol:

1. Carefully remove the chip carrier from the foil packaging. Note: Avoid contact with the carrier sippers.

2. Insert the chip carrier into the Maverick Diagnostic System.

3. Prepare a room temperature reagent plate.

4. “Flick” the plate to ensure all reagents are on the bottom of the wells.

5. A Conditioner sample (any serum sample) is diluted in the running buffer that is preloaded into the reagent plate. Pierce foil and add 10 μL of the initialization sample to wells A1 and A2 of the reagent plate. Mix well by pipetting up and down 10 times using a pipette with a set volume of 50 μL.

6. Scan the barcode on the reagent plate into the Maverick. Click “OK”.

7. Scan the conditioner barcode on the vial. Click “Use same ID in both channels”.

8. Load the reagent plate into the instrument by orienting the plate with the notches toward the instrument, and blue line toward the operator. Slide the reagent plate into the plate carriage, notched end first. Note: To avoid splashing or creating bubbles, hold the plate with both hands and ease into the instrument until the plate engages.

9. Once the reagent plate is loaded into the instrument, close the instrument door.

10. Click “Start Test”, then click “Yes” to start the run. Note: Once the Maverick is running, do not open the door until the assay is complete.

11. A timer will be displayed on screen counting down until the protocol is complete. The protocol will run for approximately 20 minutes.

12. Once the software indicates that the run is complete, remove the consumed reagent plate and discard. Leave the chip in the carrier in the instrument.

13. Proceed to Pregen by clicking “New Test”.

Pregen Protocol:

1. Prepare a room temperature Pregen reagent plate.

2. “Flick” the plate to ensure all reagents are on the bottom of the wells.

3. Load the Pregen reagent plate into the instrument by orienting the plate with the notches toward the instrument, and holes toward the operator. Slide the reagent plate into the plate carriage, notched end first. Note: To avoid splashing or creating bubbles, hold the plate with both hands and ease into the instrument until the plate engages.

4. Once the reagent plate is loaded into the instrument, close the instrument door.

5. Scan the QR code on the foil pouch. This will display a prompt asking if you want to continue with the test as a maintenance protocol is selected. Click “Yes”.

6. Start the run by selecting “Yes.” Note: Once the Maverick is running, do not open the door until the assay is complete.

7. A timer will be displayed on screen counting down until the Pregen protocol is complete. The Pregen protocol will run for approximately 10 minutes.

8. Once the software indicates that the run is complete, remove the consumed pregen plate and discard. Leave the chip in the carrier in the instrument.

9. Proceed to calibration by clicking “New Test”.

Calibration Protocol:

1. Prepare a room temperature reagent plate.

2. “Flick” the plate to ensure all reagents are on the bottom of the wells.

3. Calibrators are diluted in the running buffer that is preloaded into the reagent plate. Pierce foil and add 10 μL of the calibrator to wells A1 and A2 of the reagent plate. Mix well by pipetting up and down 10 times using a pipette with a set volume of 50 μL.

4. Scan the barcode on the reagent plate into the Maverick. Click “OK”.

5. Scan the calibrator barcode on the vial. Click “Use same ID in both channels”.

6. Load the reagent plate into the instrument by orienting the plate with the notches toward the instrument, and blue line toward the operator. Slide the reagent plate into the plate carriage, notched end first. Note: To avoid splashing or creating bubbles, hold the plate with both hands and ease into the instrument until the plate engages.

7. Once the reagent plate is loaded into the instrument, close the instrument door.

8. Click “Start Test”, then click “Yes” to start the run. Note: Once the Maverick is running, do not open the door until the assay is complete.

9. Once calibration is complete, the CLS monitor reviews the calibration data to ensure that the values for IgG and IgM reactivity to SARS-CoV-2 proteins are within acceptable limits. The acceptable ranges for SARS-CoV-2 reactivity from the calibrator are listed in Table 3. According to common statistical practices, the calibrator preferably has greater or equal to 7 of the 10 measurements within range to pass (IgG to 5 proteins and IgM to 5 proteins).

TABLE 3
Acceptable ranges for SARS-CoV-2 Reactivity from the Calibrator

| | CoV-2 S1 | | | CoV-2 | |
|---|---|---|---|---|---|
| | RBD | CoV-2 S1 | CoV-2 S2 | S1 + S2 | CoV-2 N |
| IgG low | 84 | 41 | 93 | 45 | 151 |
| IgG high | 209 | 92 | 162 | 101 | 321 |
| IgM low | 142 | 39 | 6 | 9 | 13 |
| IgM high | 249 | 92 | 28 | 26 | 36 |

10. If calibration does not pass, the CLS monitoring the assay and instrument performance can decide to rerun calibration as they deem appropriate. Based upon available data, the CLS monitor will decide if the calibration run(s) meet acceptability threshold and pass, or if the calibration run(s) fail.

11. Once calibration has passed, proceed to QC protocol.

12. If calibration has failed, remove and discard the chip. Obtain a new reagent plate and chip, scan the kit barcode, and start the sample initialization protocol.

External Control Protocol

1. Prepare a room temperature reagent plate.

2. “Flick” the plate to ensure all reagents are on the bottom of the wells.

3. Scan or manually enter the control ID into the Maverick—Channel 1 and 2.

4. External control samples are diluted in the running buffer that is preloaded in the reagent plate. Pierce foil and add 10 μL of external control (Positive Control-1 or Negative Control) to wells A1 and A2 of the reagent plate. Mix well by pipetting up and down 10 times using a pipette with a set volume of 50 μL.

5. Scan the barcode on the reagent plate into the Maverick.

6. Load the reagent plate into the instrument by orienting the plate with the notches toward the instrument, and blue line toward the operator. Slide the reagent plate into the plate carriage, notched end first. Note: To avoid splashing or creating bubbles, hold the plate with both hands and ease into the instrument until the plate engages.

7. Once the reagent plate is loaded into the instrument, close the instrument door.

8. Click start. Note: Once the Maverick is running, do not open the door until the assay is complete.

9. Refer to lot specific CoA for expected results.

10. Repeat steps 1-8 for the second external control (a positive and negative control preferably is run in each channel).

11. Once QC has passed the chip is now ready for use with reagent plates and approved specimen types.

12. If QC does not pass, the CLS monitoring assay and instrument performance can decide to rerun either the PC-1 or NC as they deem appropriate (similar to any PC-1 or NC on an instrument with another assay). Based upon available data, the CLS monitor will decide if the QC run(s) meet acceptability threshold and pass, or if the QC run(s) fail.

13. If QC has failed, remove and discard the chip. Obtain a new reagent plate and chip, scan the kit barcode, and start the sample initialization protocol.

Patient Sample Preparation

1. Prepare a room temperature reagent plate.

2. “Flick” the plate to ensure all reagents are on the bottom of the wells.

3. Scan or manually enter the specimen ID into the Maverick—Channel 1 field.

4. Patient sample for analysis is diluted in the running buffer that is preloaded in the reagent plate. Pierce foil and add 20 μL of whole blood (EDTA anticoagulant) or 10 μL of plasma or serum to well A1 of the reagent plate. Mix well by pipetting up and down 10 times using a pipette with a set volume of 50 μL.

5. Scan or manually enter the specimen ID into the Maverick—Channel 2 field.

6. Add 20 μL of whole blood with EDTA anticoagulant or 10 μL of plasma or serum to well A2 of the reagent plate. Mix well by pipetting up and down 10 times using a pipette with a set volume of 50 μL.

7. Scan the barcode on the reagent plate into the Maverick.

8. Load the reagent plate into the instrument by orienting the plate with the notches toward the instrument, and blue line toward the operator. Slide the reagent plate into the plate carriage, notched end first. Note: To avoid splashing or creating bubbles, hold the plate with both hands and ease into the instrument until the plate engages

9. Once the reagent plate is loaded into the instrument, close the instrument door.

10. Click Start. Note: Once the Maverick is running, do not open the door until assay is complete.

Run Completion

1. Once the assay has been completed, a status message will show “Test Complete”.

2. Open the door, remove and discard the reagent plate as a biohazard in accordance with local, state, and federal regulations.

3. The chip in the carrier may be reused for multiple assay runs (limit of 25 per channel).

4. When applicable, dispose of the chip in the carrier as a biohazard in accordance with local, state, and federal regulations.

### Example 4: Interpretation of Data

An exemplary output plot of the detection of resonance wavelength change across the process of applying a biological sample comprising anti-SARS-CoV-2 antibodies, washing for a first time, application of anti-human IgG to bind to human anti-SARS-CoV-2 IgG, washing for a second time, and application of anti-human IgM to bind to human anti-SARS-CoV-2 IgM in a ring resonator device is depicted in FIG. 9.

A multiplexed SARS-CoV-2 antibody detection assay using the five SARS-CoV-2 antigens shown in Table 2 was performed. A sample is determined to be from a patient who produces anti-SARS-CoV-2 antibodies, likely from a previous SARS-CoV-2 infection, if any two or more antigen/ring resonator outputs is determined to be above the specified cutoff shown in Table 4. Note that because both IgG and IgM are tested, there are ten total combinations of SARS-CoV-2 antigens and associated antibodies from which the any two or more antigen/ring resonator outputs can be selected.

TABLE 4
SARS-CoV-2 immunoassay cutoff values

| | Antigen | | | | |
|---|---|---|---|---|---|
| | CoV-2 S1 | | | CoV-2 | |
| | RBD | CoV-2 S1 | CoV-2 S2 | S1 + S2 | CoV-2 N |
| IgG | 11 | 10 | 13 | 24 | 10 |
| IgM | 22 | 10 | 10 | 11 | 10 |

In some implementations of the devices disclosed herein, the immunoassay utilizes a multi-analyte analysis algorithm (MAAA) to make the determination on patient samples of positive or negative or indeterminate for antibodies to the SARS-CoV-2 virus. The algorithm employed is an ensemble method called Random Forests Classification. Random Forests contain a number of decision trees constructed from randomly chosen features that each make predictions on the data set, the aggregation of which gives the final result. These models are capable of fitting complex datasets and are resistant to overfitting.

The implementation of the methods used herein uses 3000 such decision trees sampled randomly from training data and are validated against test data. The model was also cross validated using five-fold cross validation. Three models were trained, and the combined IgG and IgM model proved to be the most robust to call patient samples positive, negative, or indeterminate for antibodies to SARS-CoV-2. The scoring criteria is depicted in Table 5.

TABLE 5
Scoring criteria

| Probability of | | |
|---|---|---|
| positive score | Result | Test result interpretation |
| 0.55-1.00 | Positive | Anti-SARS-CoV-2 antibodies |
| | | are detected |
| 0.451-0.549 | Indeterminate | |
| 0.0-0.45 | Negative | Anti-SARS-CoV-2 antibodies |
| | | are not detected. |

Training of algorithm: 755 presumptively normal samples collected prior to November 2019 and 243 samples that were confirmed PCR positive for SARS-CoV-2 from 243 subjects were used to train the MAAA. All negative samples were collected from rheumatology or primary care clinics in routine clinical care, under IRB. All positive samples were collected retrospectively from patients presenting in ambulatory clinics, with suspected COVID-19, and who underwent NP/OP swab confirmation of SARS-CoV-2.

Known SARS-CoV-2 positive samples: 338 serum and plasma samples that were not used in MAAA training collected from 275 patients confirmed PCR positive for SARS-CoV-2 were tested using the SARS-CoV-2 Multi-Antigen Serology Panel. Samples were collected prospectively or retrospectively, and all patients were confirmed to be SARS-CoV-2 positive by PCR. Table 6 presents positive percent agreement by time from a positive PCR test. No more than one sample was collected from each patient at each time period.

TABLE 6
Validation of MAAA

| Days from | Number | | Positive Percent | 95% Confidence |
|---|---|---|---|---|
| symptom onset | tested | Positive | Agreement (PPA) | Interval |
| 0-7 | 69 | 46 | 66.67% | 54.93%-76.65% |
| 8-14 | 88 | 80 | 90.91% | 83.07%-95.32% |
| ≥15 | 181 | 174 | 96.13% | 92.23%-98.11% |
| Total | 338 | | | |

Presumptively SARS-CoV-2 negative samples: 814 presumptively normal samples collected prior to November 2019 were utilized to validate the MAAA. These samples were independent from the training sample set, but collected from a similar patient cohort. In addition, 48 samples collected from patients confirmed to be SARS-CoV-2 negative by PCR were evaluated for a total of 862 negative samples tested. The number of samples tested was 862, and 842 resulted in a negative result. The negative percent agreement (NPA) was 97.68%, and the 95% confidence interval was 96.44%-98.49%.

Matrix comparison: A total of 31 K2EDTA anticoagulated whole blood, plasma, and serum pairs, collected from patients at the same time tested in duplicates were compared: Data was generated by the random forest machine learning algorithm as to overall positivity (combined IgG and IgM in model). Samples with low and high probabilities of positive results were included. The study supports equivalency of serum, K2EDTA whole blood, and K2EDTA plasma as matrices for samples tested with the Maverick SARS-CoV-2 Multi-Antigen Serology Panel. The comparison results for serum vs. K2EDTA whole blood is shown in Table 7, and the comparison results for serum vs. K2EDTA plasma is shown in Table 8.

TABLE 7
Serum vs. K2ETA whole blood comparison

| | Serum | Serum | Serum | |
|---|---|---|---|---|
| | Positive | Indeterminate | Negative | Total |
| Whole blood | 39 | 0 | 0 | 39 |
| Positive | | | | |
| Whole blood | 1 | 2 | 0 | 3 |
| Indeterminate | | | | |
| Whole blood | 0 | 0 | 20 | 20 |
| Negative | | | | |
| Total | 40 | 2 | 20 | |

When Indeterminate is considered positive, PPA is 100% and NPA is 100%.
When Indeterminate is considered negative, PPA is 98% and NPA is 100%.

TABLE 8
Serum vs. K2EDTA plasma comparison

| | Serum | Serum | Serum | |
|---|---|---|---|---|
| | Positive | Indeterminate | Negative | Total |
| Plasma Positive | 40 | 0 | 0 | 40 |
| Plasma | 0 | 2 | 1 | 3 |
| Indeterminate | | | | |
| Plasma Negative | 0 | 0 | 19 | 19 |
| Total | 40 | 2 | 20 | |

When Indeterminate is considered positive, PPA is 100% and NPA is 95%.
When Indeterminate is considered negative, PPA is 100% and NPA is 100%.

The Maverick SARS-CoV-2 Multi-Antigen Serology Panel was evaluated for cross-reactivity in patients with autoimmune disease and in patients with infections with non-SARS-CoV-2 viruses. No cross reactivity was observed with the diseases listed in Table 9.

TABLE 9
Cross reactivity tests of patients with autoimmune
diseases and non-SARS-CoV-2 viruses

| | N of | Number | Number |
|---|---|---|---|
| Condition | samples | positive | negative |
| Systemic lupus erythematosus | 5 | 0 | 5 |
| Rheumatoid arthritis | 5 | 0 | 5 |
| Mixed connective tissue disease | 5 | 0 | 5 |
| Scleroderma | 5 | 0 | 5 |
| Osteoporosis | 5 | 0 | 5 |
| Respiratory syncytial virus | 5 | 0 | 5 |
| Cytomegalovirus | 4 | 0 | 4 |
| Epstein Barr virus | 3 | 0 | 3 |
| Hepatitis B virus | 3 | 0 | 3 |
| Hepatitis C virus | 4 | 0 | 4 |

### Additional Examples

1. A method of performing a multiplexed immunoassay for detecting multiple antigens, comprising:

(a) obtaining a biological sample comprising immunoglobulins;

(b) providing a substrate comprising a fluidic channel, wherein a plurality of different antigens are attached to the fluidic channel at respectively different loci in the fluidic channel;

(c) flowing the biological sample through the fluidic channel under conditions that permit immunoglobulins in the biological sample to bind to an antigen attached to the fluidic channel;

(d) flowing a wash buffer through the fluidic channel to remove immunoglobulins that do not bind to an antigen or that bind to an antigen with weak affinity from the fluidic channel;

(e) flowing a first probe specific for a first immunoglobulin type through the fluidic channel under conditions that permit the first probe to bind to first immunoglobulins that are bound to the antigens attached to the fluidic channel.

2. The method of example 1, further comprising:

(f) detecting a signal indicative of the presence or absence of immunoglobulins of the first immunoglobulin type that are specific for an antigen.

3. The method of example 2, wherein the biological sample is from a subject and further comprising:

(g) determining, based on the presence or absence of immunoglobulins of the first immunoglobulin type that are specific for an antigen, whether or not the subject has an infection or immune disorder of interest and/or whether or not the subject has a second condition, wherein the plurality of different antigens are selected to improve the specificity and/or sensitivity for the infection or immune disorder of interest.

4. A method of performing a multiplexed immunoassay for detecting multiple antigens, comprising:

(a) obtaining a biological sample comprising immunoglobulins;

(b) providing a substrate comprising a fluidic channel and a plurality of optical sensors, wherein the plurality of optical sensors is situated within the fluidic channel, and wherein the optical sensors comprise multiple copies of a single antigen, wherein a plurality of different antigens are attached to different optical sensors in the plurality of optical sensors;

(c) flowing the biological sample through the fluidic channel to contact the biological sample with the plurality of optical sensor, under conditions that permit immunoglobulins in the biological sample to bind to an antigen of an optical sensor;

(d) flowing a wash buffer through the fluidic channel to remove immunoglobulins that do not bind to an antigen or that bind to an antigen with weak affinity from the plurality of optical sensors;

(e) flowing a first probe specific for a first immunoglobulin type through the fluidic channel under conditions that permit the first probe to bind to first immunoglobulins that are bound to the antigen of one of the optical sensors;

(f) detecting changes in an optical property of optical sensors in the plurality of optical sensors during the flowing steps of at least (c) and (e), and optionally (d).

5. The method of example 4, further comprising:

(g) determining, based on the detected changes in optical property of the optical sensors in the plurality of optical sensors, the presence or absence of immunoglobulins of the first immunoglobulin type that are specific for an antigen.

6. The method of example 5, wherein the biological sample is from a subject and further comprising:

(h) determining, based on the presence or absence of immunoglobulins of the first immunoglobulin type that are specific for an antigen, whether or not the subject has an infection or immune disorder, wherein the plurality of different antigens are selected to improve the specificity and/or sensitivity for the infection or immune disorder.

7. The method of any one of examples 4-6, wherein the plurality of optical sensors comprises 2, 3, 4, 5, 6, 7, 8, 9, 10, 11, 12, 13, 14, 15, 16, 17, 18, 19, 20, 21, 22, 23, 24, 25, 26, 27, or 28 optical sensors.

8. The method of any one of examples 1-7, wherein the first immunoglobulin type is IgG, IgM, IgA, IgD, or IgE.

9. The method of any one of examples 1-8, wherein the determining the presence or absence of immunoglobulins of the first immunoglobulin type that are specific for an antigen comprises quantitatively determining the amount of the first immunoglobulins that are specific for an antigen.

10. A method of performing a multiplexed immunoassay for detecting multiple antigens, comprising:

(a) obtaining a biological sample comprising immunoglobulins;

(b) providing a substrate comprising a fluidic channel, wherein a plurality of different antigens are attached to the fluidic channel;

(c) flowing the biological sample through the fluidic channel under conditions that permit the immunoglobulins in the biological sample to bind to an antigen attached to the fluidic channel at respectively different loci in the fluidic channel;

(d) flowing a wash buffer through the fluidic channel to remove immunoglobulins that do not bind to an antigen or that bind to an antigen with weak affinity from the loci in the fluidic channel;

(e) flowing a first probe specific for a first immunoglobulin type through the fluidic channel under conditions that permit the first probe to bind to first immunoglobulins that are bound to the antigens of the loci in the fluidic channel;

(f) flowing a second probe specific for a second immunoglobulin type through the fluidic channel under conditions that permit the second probe to bind to second immunoglobulins that are bound to the antigens of the loci in the fluidic channel.

11. The method of example 10, further comprising:

(g) detecting the presence or absence of immunoglobulins of the first immunoglobulin type or second immunoglobulin type that are specific for an antigen.

12. The method of example 11, wherein the biological sample is from a subject and further comprising:

(h) determining, based on the presence or absence of immunoglobulins of the first immunoglobulin type that are specific for an antigen, whether or not the subject has an infection or immune disorder, wherein the plurality of different antigens are selected to improve the specificity and/or sensitivity for the infection or immune disorder.

13. A method of performing a multiplexed immunoassay for detecting multiple antigens, comprising:

(a) obtaining a biological sample comprising immunoglobulins;

(b) providing a substrate comprising a fluidic channel and a plurality of optical sensors, wherein the plurality of optical sensors is situated within the fluidic channel, and wherein the optical sensors comprise multiple copies of a single antigen and wherein a plurality of different antigens are attached to different optical sensors in the plurality of sensors;

(c) flowing the biological sample through the fluidic channel to contact the biological sample with the plurality of optical sensors, under conditions that permit the immunoglobulins in the biological sample to bind to an antigen of an optical sensor;

(d) flowing a wash buffer through the fluidic channel to remove immunoglobulins that do not bind to an antigen or that bind to an antigen with weak affinity from the plurality of optical sensors;

(e) flowing a first probe specific for a first immunoglobulin type through the fluidic channel under conditions that permit the first probe to bind to first immunoglobulins that are bound to the antigen of one of the optical sensors;

(f) flowing a second probe specific for a second immunoglobulin type through the fluidic channel under conditions that permit the second probe to bind to second immunoglobulins that are bound to the antigen of one of the optical sensors;

(g) detecting changes in an optical property of optical sensors in the plurality of optical sensors during the flowing steps of at least (c), (e) and (f), and optionally (d).

14. The method of example 13, further comprising:

(h) determining, based on the detected changes in the optical property of the optical sensors in the plurality of optical sensors, the presence or absence of immunoglobulins of the first immunoglobulin type or second immunoglobulin type that are specific for an antigen.

15. The method of example 14, wherein the biological sample is from a subject and further comprising:

(i) determining, based on the presence or absence of immunoglobulins of the first immunoglobulin type that are specific for an antigen, whether or not the subject has an infection or immune disorder, wherein the plurality of different antigens are selected to improve the specificity and/or sensitivity for the infection or immune disorder.

16. The method of any one of examples 13-15, wherein the plurality of optical sensors comprises 2, 3, 4, 5, 6, 7, 8, 9, 10, 11, 12, 13, 14, 15, 16, 17, 18, 19, 20, 21, 22, 23, 24, 25, 26, 27, or 28 optical sensor.

17. The method of any one of examples 10-16, wherein the first immunoglobulin type is IgG, IgM, IgA, IgD, or IgE, and the second immunoglobulin type is IgM, IgG, IgA, IgD, or IgE.

18. The method of any one of examples 10-17, wherein the determining the presence or absence of immunoglobulins of the first immunoglobulin type or second immunoglobulin type that are specific for an antigen comprises quantitatively determining the amount of the first immunoglobulins or second immunoglobulins, respectively, that are specific for an antigen.

19. The method of any one of examples 1-18, wherein the infection or immune disorder is a viral infection.

20. The method of example 19, wherein the viral infection is a coronavirus infection.

21. The method of example 20, wherein the coronavirus infection is a SARS-CoV-2 infection, and the plurality of antigens comprises at least one immunogenic peptide fragment of a SARS-CoV-2 protein selected from the group consisting of the S protein, M protein, N protein, E protein, and HE protein.

22. The method of example 19, wherein the viral infection is an influenza infection.

23. The method of any one of examples 1-22, wherein the biological sample is whole blood, plasma, or serum.

24. The method of any one of examples 1-23, wherein the biological sample is provided in a volume of 250 μL or less, such as 10, 20, 30, 40, 50, 60, 70, 80, 90, 100, 110, 120, 130, 140, 150, 160, 170, 180, 190, 200, 210, 220, 230, 240, or 250 μL, or any volume within a range defined by any two aforementioned volumes.

25. The method of any one of examples 1-24, wherein the method is performed within 60 minutes or less, such as 5, 10, 11, 12, 13, 14, 15, 16, 17, 18, 19, 20, 25, 30, 35, 40, 45, 50, 55, or 60 minutes, or any time duration within a range defined by any two aforementioned values.

26. The method of any one of examples 1-25, wherein the plurality of antigens comprises at least one antigen specific for the infection or immune disorder and at least one antigen specific for a second condition.

27. The method of example 28, wherein the at least one antigen specific for the infection or immune disorder is an antigen specific for SARS-CoV-2, and wherein the at least one antigen specific for a second condition is an antigen specific for a virus selected from the group consisting of non-SARS-CoV-2 coronavirus, influenza virus, and combinations thereof.

28. The method of any one of examples 1-27, wherein the plurality of antigens comprises at least one antigen with high specificity for an immunoglobulin associated with an infection or immune disorder and at least one antigen with high sensitivity for an immunoglobulin associated with the infection or immune disorder.

29. The method of any one of examples 1-28, wherein the plurality of antigens comprises two or more antigens with high specificity for an immunoglobulin associated with an infection or immune disorder and two or more antigens with high sensitivity for an immunoglobulin associated with the infection or immune disorder.

30. The method of any one of examples 1-29, further comprising combining the measured amount of antigens with different sensitivities for immunoglobulins associated with an infection or immune disorder and the measured amount of antigens with different specificities for immunoglobulins associated with an infection or immune disorder.

31. The method of example 30, wherein the combined measurements provide an overall sensitivity and specificity for an infection or immune disorder.

32. The method of example 28, wherein the infection or immune disorder is a SARS-CoV-2 infection and the at least one antigen with high specificity comprises at least 1, 2, 3, 4, 5, 6, 7, 8, 9, or 10 antigens with protein sequences unique to SARS-CoV-2, and the at least one antigen with high sensitivity comprises at least 1, 2, 3, 4, 5, 6, 7, 8, 9, or 10 antigens with protein sequences that are highly immunogenic but common in Coronaviridae with at least 50%, 60%, 70%, 80%, 85%, 90%, 91%, 92%, 93%, 94%, 95%, 96%, 97%, 98%, 99%, or 100% homology.

33. The method of example 28, wherein the infection or immune disorder is a coronavirus infection and the at least one antigen with high specificity comprises at least 1, 2, 3, 4, 5, 6, 7, 8, 9, or 10 antigens with protein sequences unique to a non-SARS-CoV-2 coronavirus, and the at least one antigen with high sensitivity comprises at least 1, 2, 3, 4, 5, 6, 7, 8, 9, or 10 antigens with protein sequences that are highly immunogenic but common in Coronaviridae with at least 50%, 60%, 70%, 80%, 85%, 90%, 91%, 92%, 93%, 94%, 95%, 96%, 97%, 98%, 99%, or 100% homology.

34. The method of example 32, wherein the presence of immunoglobulins that are specific for an antigen with high specificity reduces a false positive reading of a SARS-CoV-2 infection.

35. The method of example 32, wherein the presence of immunoglobulins that are specific for an antigen with high sensitivity reduces a false negative reading of a SARS-CoV-2 infection.

36. The method of any of the examples above, further comprising detecting changes in an optical sensor to determine the presence of a biological molecule.

37. The method of any of the examples above, wherein the optical sensor comprises an optical resonator.

38. The method of any of the examples above, wherein the optical sensor comprises an optical ring resonator.

39. The method of any of the examples above, wherein an optical property of the optical sensor is detected to determine the presence of a biological molecule.

40. The method of any of the examples above, wherein the optical property detected comprises the resonance wavelength.

41. The method of any of the examples above, further comprising detecting changes in an optical property of optical sensors in the plurality of optical sensors during the flowing step of step (d).

42. The method of any of the examples above, wherein the optical sensor comprises a waveguide-based optical sensor.

43. The method of any of the examples above, wherein the optical sensor comprises a waveguide.

Second set of additional examples:

1. A method of performing a multiplexed immunoassay for detecting multiple antigens, comprising:

(a) flowing a biological sample comprising immunoglobulins from a subject through a fluidic channel of a substrate under conditions that permit immunoglobulins in the biological sample to bind to an antigen attached to the fluidic channel, wherein a plurality of different antigens are attached to the fluidic channel at respectively different loci in the fluidic channel;

(b) flowing a first probe specific for a first immunoglobulin type through the fluidic channel under conditions that permit the first probe to bind to first immunoglobulins that are bound to the antigens attached to the fluidic channel.

2. The method of example 1, further comprising:

flowing a wash buffer through the fluidic channel to remove immunoglobulins that do not bind to an antigen or that bind to an antigen with weak affinity from the fluidic channel after the step of (a) and before the step of (b).

3. The method of example 1 or 2, further comprising:

(c) detecting a signal indicative of the presence or absence of immunoglobulins of the first immunoglobulin type that are specific for an antigen.

4. The method of any one of examples 1-3, further comprising:

(d) determining, based on the presence or absence of immunoglobulins of the first immunoglobulin type that are specific for an antigen, whether or not the subject has an infection or immune disorder of interest and/or whether or not the subject has a second condition, wherein the plurality of different antigens are selected to improve the specificity and/or sensitivity for the infection or immune disorder of interest.

5. A method of performing a multiplexed immunoassay for detecting multiple antigens, comprising:

(a) providing a substrate comprising a fluidic channel and a plurality of optical sensors, wherein the plurality of optical sensors is situated within the fluidic channel, and wherein the optical sensors comprise multiple copies of a single antigen, wherein a plurality of different antigens are attached to different optical sensors in the plurality of optical sensors;

(b) flowing a biological sample comprising immunoglobulins from a subject through the fluidic channel to contact the biological sample with the plurality of optical sensor, under conditions that permit immunoglobulins in the biological sample to bind to an antigen of an optical sensor;

(c) flowing a first probe specific for a first immunoglobulin type through the fluidic channel under conditions that permit the first probe to bind to first immunoglobulins that are bound to the antigen of one of the optical sensors;

(d) detecting changes in an optical property of optical sensors in the plurality of optical sensors during the flowing steps of at least (b) and (c).

6. The method of example 5, further comprising flowing a wash buffer through the fluidic channel to remove immunoglobulins that do not bind to an antigen or that bind to an antigen with weak affinity from the plurality of optical sensors after the step of (b) and before the step of (c).

7. The method of example 6, further comprising detecting changes in an optical property of optical sensors in the plurality of optical sensors during the flowing of the wash buffer.

8. The method of any one of examples 5-7, further comprising:

(e) determining, based on the detected changes in optical property of the optical sensors in the plurality of optical sensors, the presence or absence of immunoglobulins of the first immunoglobulin type that are specific for an antigen.

9. The method of example 8, further comprising:

(f) determining, based on the presence or absence of immunoglobulins of the first immunoglobulin type that are specific for an antigen, whether or not the subject has an infection or immune disorder, wherein the plurality of different antigens are selected to improve the specificity and/or sensitivity for the infection or immune disorder.

10. The method of any one of examples 5-9, wherein the plurality of optical sensors comprises 2, 3, 4, 5, 6, 7, 8, 9, 10, 11, 12, 13, 14, 15, 16, 17, 18, 19, 20, 21, 22, 23, 24, 25, 26, 27, or 28 optical sensors.

11. The method of any one of examples 1-10, wherein the first immunoglobulin type is IgG, IgM, IgA, IgD, or IgE.

12. The method of any one of examples 1-11, wherein the determining the presence or absence of immunoglobulins of the first immunoglobulin type that are specific for an antigen comprises quantitatively determining the amount of the first immunoglobulins that are specific for an antigen.

13. A method of performing a multiplexed immunoassay for detecting multiple antigens, comprising:

(a) flowing a biological sample comprising immunoglobulins from a subject through a fluidic channel of a substrate under conditions that permit the immunoglobulins in the biological sample to bind to an antigen attached to the fluidic channel at respectively different loci in the fluidic channel, wherein a plurality of different antigens are attached to the fluidic channel;

(b) flowing a first probe specific for a first immunoglobulin type through the fluidic channel under conditions that permit the first probe to bind to first immunoglobulins that are bound to the antigens of the loci in the fluidic channel;

(c) flowing a second probe specific for a second immunoglobulin type through the fluidic channel under conditions that permit the second probe to bind to second immunoglobulins that are bound to the antigens of the loci in the fluidic channel.

14. The method of example 13, further comprising:

flowing a wash buffer through the fluidic channel to remove immunoglobulins that do not bind to an antigen or that bind to an antigen with weak affinity from the loci in the fluidic channel after the step of (a) and before the step of (b), and/or after the step of (b) and before the step of (c).

15. The method of example 13 or 14, further comprising:

(d) detecting the presence or absence of immunoglobulins of the first immunoglobulin type or second immunoglobulin type that are specific for an antigen.

16. The method of example 15, further comprising:

(e) determining, based on the presence or absence of immunoglobulins of the first immunoglobulin type that are specific for an antigen, whether or not the subject has an infection or immune disorder, wherein the plurality of different antigens are selected to improve the specificity and/or sensitivity for the infection or immune disorder.

17. A method of performing a multiplexed immunoassay for detecting multiple antigens, comprising:

(a) providing a substrate comprising a fluidic channel and a plurality of optical sensors, wherein the plurality of optical sensors is situated within the fluidic channel, and wherein the optical sensors comprise multiple copies of a single antigen and wherein a plurality of different antigens are attached to different optical sensors in the plurality of sensors;

(b) flowing a biological sample comprising immunoglobulins from a subject through the fluidic channel to contact the biological sample with the plurality of optical sensors, under conditions that permit the immunoglobulins in the biological sample to bind to an antigen of an optical sensor;

(c) flowing a first probe specific for a first immunoglobulin type through the fluidic channel under conditions that permit the first probe to bind to first immunoglobulins that are bound to the antigen of one of the optical sensors;

(d) flowing a second probe specific for a second immunoglobulin type through the fluidic channel under conditions that permit the second probe to bind to second immunoglobulins that are bound to the antigen of one of the optical sensors;

(e) detecting changes in an optical property of optical sensors in the plurality of optical sensors during the flowing steps of (b), (c) and (d).

18. The method of example 17, further comprising:

flowing a wash buffer through the fluidic channel to remove immunoglobulins that do not bind to an antigen or that bind to an antigen with weak affinity from the plurality of optical sensors after the step of (b) and before the step of (c), and/or after the step of (c) and before the step of (d).

19. The method of example 18, further comprising:

detecting changes in the optical property of optical sensors in the plurality of optical sensors during the flowing of the wash buffer.

20. The method of any one of examples 17-19, further comprising:

(f) determining, based on the detected changes in the optical property of the optical sensors in the plurality of optical sensors, the presence or absence of immunoglobulins of the first immunoglobulin type or second immunoglobulin type that are specific for an antigen.

21. The method of example 20, further comprising:

(g) determining, based on the presence or absence of immunoglobulins of the first immunoglobulin type that are specific for an antigen, whether or not the subject has an infection or immune disorder, wherein the plurality of different antigens are selected to improve the specificity and/or sensitivity for the infection or immune disorder.

22. The method of any one of examples 17-21, wherein the plurality of optical sensors comprises 2, 3, 4, 5, 6, 7, 8, 9, 10, 11, 12, 13, 14, 15, 16, 17, 18, 19, 20, 21, 22, 23, 24, 25, 26, 27, or 28 optical sensors.

23. The method of any one of examples 13-21, wherein the first immunoglobulin type is IgG, and the second immunoglobulin type is IgM, IgG, IgA, IgD, or IgE.

24. The method of any one of examples 13-21, wherein the determining the presence or absence of immunoglobulins of the first immunoglobulin type or second immunoglobulin type that are specific for an antigen comprises quantitatively determining the amount of the first immunoglobulins or second immunoglobulins, respectively, that are specific for an antigen.

25. The method of any one of examples 1-24, wherein the infection or immune disorder is a viral infection.

26. The method of example 25, wherein the viral infection is a coronavirus infection.

27. The method of example 26, wherein the coronavirus infection is a SARS-CoV-2 infection, and the plurality of antigens comprises at least one immunogenic peptide fragment of a SARS-CoV-2 protein selected from the group consisting of the S protein, M protein, N protein, E protein, and HE protein.

28. The method of example 25, wherein the viral infection is an influenza infection.

29. The method of any one of examples 1-28, wherein the biological sample is whole blood, plasma, or serum.

30. The method of any one of examples 1-29, wherein the biological sample is provided in a volume of 250 μL or less, such as 10, 20, 30, 40, 50, 60, 70, 80, 90, 100, 110, 120, 130, 140, 150, 160, 170, 180, 190, 200, 210, 220, 230, 240, or 250 μL, or any volume within a range defined by any two aforementioned volumes.

31. The method of any one of examples 1-30, wherein the method is performed within 60 minutes or less, such as 5, 10, 11, 12, 13, 14, 15, 16, 17, 18, 19, 20, 25, 30, 35, 40, 45, 50, 55, or 60 minutes, or any time duration within a range defined by any two aforementioned values.

32. The method of any one of examples 1-31, wherein the plurality of antigens comprises at least one antigen specific for the infection or immune disorder and at least one antigen specific for a second condition.

33. The method of example 32, wherein the at least one antigen specific for the infection or immune disorder is an antigen specific for SARS-CoV-2, and wherein the at least one antigen specific for a second condition is an antigen specific for a virus selected from the group consisting of non-SARS-CoV-2 coronavirus, influenza virus, and combinations thereof.

34. The method of any one of examples 1-33, wherein the plurality of antigens comprises at least one antigen with high specificity for an immunoglobulin associated with an infection or immune disorder and at least one antigen with high sensitivity for an immunoglobulin associated with the infection or immune disorder.

35. The method of any one of examples 1-34, wherein the plurality of antigens comprises two or more antigens with high specificity for an immunoglobulin associated with an infection or immune disorder and two or more antigens with high sensitivity for an immunoglobulin associated with the infection or immune disorder.

36. The method of any one of examples 1-35, wherein the plurality of antigens comprises antigens with different sensitivities for immunoglobulins associated with an infection or immune disorder and antigens with different specificities for immunoglobulins associated with an infection or immune disorder.

37. The method of example 36, wherein the combined measurements provide an overall sensitivity and specificity for an infection or immune disorder.

38. The method of any one of examples 1-37, wherein the infection or immune disorder is a SARS-CoV-2 infection and the at least one antigen with high specificity comprises at least 1, 2, 3, 4, 5, 6, 7, 8, 9, or 10 antigens with protein sequences unique to SARS-CoV-2, and the at least one antigen with high sensitivity comprises at least 1, 2, 3, 4, 5, 6, 7, 8, 9, or 10 antigens with protein sequences that are highly immunogenic but common in Coronaviridae with at least 50%, 60%, 70%, 80%, 85%, 90%, 91%, 92%, 93%, 94%, 95%, 96%, 97%, 98%, 99%, or 100% homology.

39. The method of any one of examples 1-38, wherein the infection or immune disorder is a coronavirus infection and the at least one antigen with high specificity comprises at least 1, 2, 3, 4, 5, 6, 7, 8, 9, or 10 antigens with protein sequences unique to a non-SARS-CoV-2 coronavirus, and the at least one antigen with high sensitivity comprises at least 1, 2, 3, 4, 5, 6, 7, 8, 9, or 10 antigens with protein sequences that are highly immunogenic but common in Coronaviridae with at least 50%, 60%, 70%, 80%, 85%, 90%, 91%, 92%, 93%, 94%, 95%, 96%, 97%, 98%, 99%, or 100% homology.

40. The method of any one of examples 1-39, wherein the presence of immunoglobulins that are specific for an antigen with high specificity reduces a false positive reading of a SARS-CoV-2 infection.

41. The method of any one of examples 1-40, wherein the presence of immunoglobulins that are specific for an antigen with high sensitivity reduces a false negative reading of a SARS-CoV-2 infection.

42. The method of any of the examples above, further comprising detecting changes in an optical sensor to determine the presence of a biological molecule.

43. The method of any of the examples above, wherein the optical sensor comprises an optical resonator.

44. The method of any of the examples above, wherein the optical sensor comprises an optical ring resonator.

45. The method of any of the examples above, wherein an optical property of the optical sensor is detected to determine the presence of a biological molecule.

46. The method of any of the examples above, wherein the optical property detected comprises the resonance wavelength.

47. The method of any of the examples above, further comprising detecting changes in an optical property of optical sensors in the plurality of optical sensors during the flowing step of step (d).

48. The method of any of the examples above, wherein the optical sensor comprises a waveguide-based optical sensor.

49. The method of any of the examples above, wherein the optical sensor comprises a waveguide.

Third set of additional examples:

1. A system for detecting SARS-CoV-2 in a sample comprising:

an optical sensor; and

a SARS-CoV-2 specific antigen attached to a surface of the optical sensor, wherein the SARS-CoV-2 specific antigen is capable of binding to an immunoglobulin;

wherein said optical sensor has an optical property that is altered by said immunoglobulin bound to said SARS-CoV-2 specific antigen, such that said optical sensor is configured to sense said immunoglobulin combined with said SARS-CoV-2 specific antigen.

2. The system of Example 1, wherein the optical sensor comprises an optical resonator.

3. The system of Examples 1 or 2, wherein the optical sensor comprises an interferometric structure.

4. The system of any of Examples 1-3, wherein the optical sensor comprises an optical ring resonator.

5. The system of any of the examples above, wherein the optical sensor comprises a waveguide-based optical sensor.

6. The system of any of the examples above, wherein the optical sensor is integrated on a substrate.

7. The system of Example 6, wherein the substrate comprises a silicon substrate.

8. The system of any of the Examples 6 or 7, further comprising a fluidic channel on said substrate that is configured to flow a sample that includes said immunoglobulin across said optical channel.

9. The system of any of Examples 6-8, wherein the optical sensor is included in a plurality of optical sensors disposed on said substrate.

10. The system of any of the examples above, wherein the optical sensor is included in a plurality of optical sensors.

11. The system of any of the examples above, wherein said optical property comprises the resonance wavelength.

12. The system of any of the examples above, further comprising a detector configured to receive an output signal from said optical sensor, said output signal being altered by said altered optical property.

13. The system of any of the examples above, further comprising a processor configured to identify the presence of said SARS-CoV-2 specific immunoglobulin in said sample based on said alteration in the output signal received by said detector.

14. The system of any of the examples above, wherein the surface of said optical sensor comprises multiple copies of said SARS-CoV-2 specific antigen attached thereto.

15. The system of any of Examples 6-14, wherein a probe is capable of binding to said immunoglobulin bound to said SARS-CoV-2 specific antigen, and wherein said optical sensor has an optical property that is altered by said probe binding to said immunoglobulin bound to said SARS-CoV-2 specific antigen, such that said optical sensor is configured to sense said probe combined with said immunoglobulin and SARS-CoV-2 specific antigen.

16. The system of any of Examples 8-15, wherein said fluidic channel on said substrate is configured to flow a probe capable of binding to said immunoglobulin across said optical channel.

17. The system of Examples 15 or 16, wherein the probe is an antibody.

18. The system of Example 17, wherein said antibody is an anti-IgM antibody or an anti-IgG antibody.

19. The system of any of examples 9-18, wherein a plurality of optical sensors comprise SARS-CoV-2 specific antigens such that at least two of said plurality of optical sensors comprise different SARS-CoV-2 specific antigens.

20. The system of example 19, wherein at least one of said different SARS-CoV-2 specific antigens is substantially bound only by anti-SARS-CoV-2 immunoglobulins and not by immunoglobulins that bind a non-SARS-CoV-2 coronaviridae antigen.

21. A system for detecting an indicator of disease in a sample comprising:

an optical sensor; and

an antigen attached to a surface of the optical sensor, wherein the antigen is capable of binding to an immunoglobulin;

wherein said optical sensor has an optical property that is altered by said immunoglobulin bound to said antigen, such that said optical sensor is configured to sense said immunoglobulin combined with said antigen.

22. The system of Example 21, wherein the optical sensor comprises an optical resonator.

23. The system of Examples 21 or 22, wherein the optical sensor comprises an interferometric structure.

24. The system of any of Examples 21-23, wherein the optical sensor comprises an optical ring resonator.

25. The system of any of Examples 21-24, wherein the optical sensor comprises a waveguide-based optical sensor.

26. The system of any of Examples 21-25, wherein the optical sensor is integrated on a substrate.

27. The system of Example 26, wherein the substrate comprises a silicon substrate.

28. The system of any of the Examples 26 or 27, further comprising a fluidic channel on said substrate that is configured to flow a sample that includes said immunoglobulin across said optical channel.

29. The system of any of Examples 26-28, wherein the optical sensor is included in a plurality of optical sensors disposed on said substrate.

30. The system of any of Examples 21-29, wherein the optical sensor is included in a plurality of optical sensors.

31. The system of any of Examples 21-30, wherein said optical property comprises the resonance wavelength.

32. The system of any of Examples 21-31, further comprising a detector configured to receive an output signal from said optical sensor, said output signal being altered by said altered optical property.

33. The system of any of Examples 21-32, further comprising a processor configured to identify the presence of said antigen specific immunoglobulin in said sample based on said alteration in the output signal received by said detector.

34. The system of any of Examples 21-33, wherein the surface of said optical sensor comprises multiple copies of said antigen attached thereto.

35. The system of any of Examples 26-34, wherein a probe is capable of binding to said immunoglobulin bound to said antigen, and wherein said optical sensor has an optical property that is altered by said probe binding to said immunoglobulin bound to said antigen, such that said optical sensor is configured to sense said probe combined with said immunoglobulin and antigen.

36. The system of any of Examples 26-35, wherein said fluidic channel on said substrate is configured to flow a probe capable of binding to said immunoglobulin across said optical channel.

37. The system of Examples 35 or 36, wherein the probe is an antibody.

38. The system of Example 37, wherein said antibody is an anti-IgM antibody or an anti-IgG antibody.

39. The system of any of examples 29-37, wherein a plurality of optical sensors of said comprise antigens such that at least two of said plurality of optical sensors comprise different antigens.

40. The system of any of examples 21-38, wherein the disease is a viral infection or an immune disorder.

Fourth set of additional examples:

1. A method for detecting SARS-CoV-2 in a sample comprising:

providing an optical sensor comprising a SARS-CoV-2 antigen attached to a surface of the optical sensor, wherein the SARS-CoV-2 antigen is capable of binding to an immunoglobulin;

applying a sample for which the presence or absence of the immunoglobulin is to be determined to the optical sensor under conditions in which the immunoglobulin, when present, binds with the SARS-CoV-2 antigen, wherein binding between the immunoglobulin and the SARS-CoV-2 antigen alters an optical property of the optical sensor; and

determining the presence or absence of the immunoglobulin by detecting the altered optical property of the optical sensor.

2. The method of Example 1, wherein the optical sensor comprises an optical resonator.

3. The method of Examples 1 or 2, wherein the optical sensor comprises an interferometric structure.

4. The method of any of Examples 1-3, wherein the optical sensor comprises an optical ring resonator.

5. The method of any of the examples above, wherein the optical sensor comprises a waveguide-based optical sensor.

6. The method of any of the examples above, wherein the optical sensor is integrated on a substrate.

7. The method of Example 6, wherein the substrate comprises a silicon substrate.

8. The method of any of the Examples 6 or 7, further comprising a fluidic channel on said substrate that is configured to flow a sample that includes said immunoglobulin across said optical channel.

9. The method of any of Examples 6-8, wherein the optical sensor is included in a plurality of optical sensors disposed on said substrate.

10. The method of any of the examples above, wherein the optical sensor is included in a plurality of optical sensors.

11. The method of any of the examples above, wherein said optical property comprises the resonance wavelength.

12. The method of any of the examples above, further comprising providing a detector configured to receive an output signal from said optical sensor, said output signal being altered by said altered optical property.

13. The method of any of the examples above, further comprising providing a processor configured to identify the presence of said immunoglobulin in said sample based on said alteration in the output signal received by said detector.

14. The method of any of the examples above, wherein the surface of said optical sensor comprises multiple copies of said SARS-CoV-2 specific antigen attached thereto.

15. The method of any of examples 6-14, wherein a probe is capable of binding to said immunoglobulin bound to said SARS-CoV-2 specific antigen, and wherein said optical sensor has an optical property that is altered by said probe binding to said immunoglobulin bound to said SARS-CoV-2 specific antigen, such that said optical sensor is configured to sense said probe combined with said immunoglobulin and SARS-CoV-2 specific antigen.

16. The method of any of examples 8-15, wherein said fluidic channel on said substrate is configured to flow a probe capable of binding said immunoglobulin across said optical channel.

17. The method of examples 15 or 16, wherein the probe is an antibody.

18. The method of example 17, wherein said antibody is an anti-IgM antibody or an anti-IgG antibody.

19. The method of any of examples 9-17, wherein the plurality of optical sensors comprises SARS-CoV-2 specific antigens such that at least two of said optical sensors of said plurality comprise different SARS-CoV-2 specific antigens.

20. The method of example 19, wherein at least one of said different SARS-CoV-2 specific antigens is substantially bound only by anti-SARS-CoV-2 immunoglobulins and not by immunoglobulins that bind a non-SARS-CoV-2 coronavirus antigen.

21. The method of the examples above, further comprising determining whether or not the subject has or previously had an infection or immune disorder.

22. The method of example 21, wherein the determining whether or not the subject has or previously had an infection or immune disorder is performed with a machine learning algorithm.

23. The method of example 22, wherein the machine learning algorithm is a random forest machine learning algorithm.

Embodiments have been described in connection with the accompanying drawings. However, it should be understood that the figures are not necessarily drawn to scale. Distances, angles, sizes, etc. are merely illustrative and do not necessarily bear an exact relationship to actual dimensions and layout of the devices illustrated. In addition, the foregoing embodiments have been described at a level of detail to allow one of ordinary skill in the art to make and use the devices, systems, etc. described herein. A wide variety of variation is possible. Components, elements, and/or steps may be altered, added, removed, or rearranged. While certain embodiments have been explicitly described, other embodiments will become apparent to those of ordinary skill in the art based on this disclosure.

Some of the systems and methods described herein can advantageously be implemented, at least in part, using, for example, computer software, hardware, firmware, or any combination of software, hardware, and firmware. Software modules can comprise computer executable code for performing the functions described herein. In some embodiments, computer-executable code is executed by one or more general purpose computers. However, a skilled artisan will appreciate, in light of this disclosure, that any module that can be implemented using software to be executed on a general purpose computer can also be implemented using a different combination of hardware, software, or firmware. For example, such a module can be implemented completely in hardware using a combination of integrated circuits. Alternatively or additionally, such a module can be implemented completely or partially using specialized computers designed to perform the particular functions described herein rather than by general purpose computers. In addition, where methods are described that are, or could be, at least in part carried out by computer software, it should be understood that such methods can be provided on computer-readable media (e.g., optical disks such as CDs or DVDs, hard disk drives, flash memories, diskettes, or the like) that, when read by a computer or other processing device, cause it to carry out the method.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description or claims, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method of performing a multiplexed immunoassay for detecting multiple antigens, comprising:
(a) obtaining a biological sample comprising immunoglobulins;
(b) providing a substrate comprising a fluidic channel, wherein a plurality of different antigens are attached to the fluidic channel at respectively different loci in the fluidic channel;
(c) flowing the biological sample through the fluidic channel under conditions that permit immunoglobulins in the biological sample to bind to an antigen attached to the fluidic channel;
(d) flowing a wash buffer through the fluidic channel to remove immunoglobulins that do not bind to an antigen or that bind to an antigen with weak affinity from the fluidic channel;
(e) flowing a first probe specific for a first immunoglobulin type through the fluidic channel under conditions that permit the first probe to bind to first immunoglobulins that are bound to the antigens attached to the fluidic channel.

2.-9. (canceled)

10. A method of performing a multiplexed immunoassay for detecting multiple antigens, comprising:
(a) obtaining a biological sample comprising immunoglobulins;
(b) providing a substrate comprising a fluidic channel, wherein a plurality of different antigens are attached to the fluidic channel;
(c) flowing the biological sample through the fluidic channel under conditions that permit the immunoglobulins in the biological sample to bind to an antigen attached to the fluidic channel at respectively different loci in the fluidic channel;
(d) flowing a wash buffer through the fluidic channel to remove immunoglobulins that do not bind to an antigen or that bind to an antigen with weak affinity from the loci in the fluidic channel;
(e) flowing a first probe specific for a first immunoglobulin type through the fluidic channel under conditions that permit the first probe to bind to first immunoglobulins that are bound to the antigens of the loci in the fluidic channel;
(f) flowing a second probe specific for a second immunoglobulin type through the fluidic channel under conditions that permit the second probe to bind to second immunoglobulins that are bound to the antigens of the loci in the fluidic channel.

11.-78. (canceled)

79. A method of performing a multiplexed immunoassay, comprising:
(a) contacting a biological sample from a subject comprising a plurality of immunoglobulins with a plurality of optical ring resonators under conditions that permit immunoglobulins to bind to a plurality of antigens, wherein each optical ring resonator of the plurality of optical ring resonators comprises multiple copies of a single antigen, such that the plurality of optical ring resonators comprises a plurality of antigens;
(b) contacting one or more probes specific to one or more immunoglobulin types with the immunoglobulins bound to the plurality of antigens on the optical ring resonators under conditions that permit the one or more probes to bind to the immunoglobulins; and
(c) detecting changes in resonance wavelength for the plurality of optical ring resonators during the contacting step of step (a), step (b), or during both contacting steps (a) and (b).

80. The method of claim 79, wherein a change in resonance wavelength for an individual optical ring resonator of the plurality of optical ring resonators comprising the multiple copies of the single antigen indicates that either (1) an immunoglobulin that specifically binds to the single antigen is present in the plurality of immunoglobulins, or (2) the immunoglobulin that specifically binds to the single antigen comprises an immunoglobulin type to which the one or more probes specifically bind, or (3) both (1) and (2).

81. (canceled)

82. The method of claim 80 or 81, wherein detecting changes in resonance wavelength during the contacting step of step (b) indicates that (2) the immunoglobulin that specifically binds to the single antigen comprises the immunoglobulin type to which the one or more probes specifically bind.

83. The method of claim 79, wherein the plurality of optical ring resonators is situated within a fluidic channel.

84. The method of claim 83, wherein the fluidic channel is situated within a substrate or device.

85. The method of claim 83, wherein the contacting step of step (a) comprises flowing the biological sample through the fluidic channel to contact the biological sample with the plurality of optical ring resonators and the contacting step of step (b) comprises flowing the one or more probes through the fluidic channel to contact the immunoglobulins bound to the plurality of antigens on the optical ring resonators.

86. The method of claim 79, further comprising a washing step between the contacting steps of step (a) and step (b), wherein immunoglobulins that do not bind to the plurality of antigens or that bind to the plurality of antigens with weak affinity are removed from the plurality of optical ring resonators.

87. The method of claim 86, further comprising detecting changes in resonance wavelength for the plurality of optical ring resonators during the washing step, or after the washing step and before step (b), or during both the washing step and after the washing step and before step (b).

88. The method of claim 86, wherein the washing step comprises flowing a wash buffer through a fluidic channel to contact the wash buffer with the plurality of immunoglobulins and the plurality of optical ring resonators.

89. The method of claim 79, wherein the plurality of optical ring resonators comprises 2-28 optical ring resonators.

90. The method of claim 79, wherein the one or more immunoglobulin types comprises IgG, IgM, IgA, IgD, or IgE, or any combination thereof.

91. The method of claim 79, wherein the one or more immunoglobulin types comprises IgG and IgM.

92. The method of claim 79, further comprising determining, based on the detected changes in resonance wavelength for the plurality of optical ring resonators, the presence or absence of immunoglobulins of the one or more immunoglobulin types that are specific for the plurality of antigens.

93. The method of claim 92, further comprising determining, based on the presence or absence of immunoglobulins of the one or more immunoglobulin types that are specific for the plurality of antigens, whether or not the subject has or previously had an infection or immune disorder.

94. The method of claim 93, wherein the plurality of antigens are selected to improve the specificity and/or sensitivity for detecting the infection or immune disorder.

95. The method of claim 93, wherein the infection or immune disorder is a viral infection.

96. The method of claim 95, wherein the viral infection is a coronavirus infection.

97. The method of claim 96, wherein the coronavirus infection is a SARS-CoV-2 infection, and the plurality of antigens comprises at least one immunogenic peptide of a SARS-CoV-2 protein.

98. The method of claim 97, wherein the SARS-CoV-2 protein is selected from the group consisting of the S protein, M protein, N protein, E protein, and HE protein.

99. The method of claim 97, wherein the SARS-CoV-2 infection is caused by a SARS-CoV-2 variant.

100. The method of claim 99, wherein the SARS-CoV-2 variant is selected from 20I/501Y.V1 (B.1.1.7), 20H/501Y.V2 (B.1.351), 20J/501Y.V3 (P.1), B.1.1.207, VUI-202102/03 (B.1.525), VUI-202101/01 (P.2), VUI-202102/01 (A.23.1), VUI 202102/04 (B.1.1.318), VUI 202103/01 (B.1.324.1), or CAL.20C (B.1.429).

101. The method of claim 95, wherein the viral infection is an influenza infection.

102. The method of claim 79, wherein the biological sample is whole blood, plasma, or serum.

103. The method of claim 79, wherein the biological sample comprises a volume of 10-250 μL.

104. The method of claim 79, wherein the method is performed within 5-60 minutes.

105. The method of claim 79, wherein the plurality of antigens comprises at least one antigen with high specificity for an immunoglobulin associated with the infection or immune disorder and at least one antigen with high sensitivity for an immunoglobulin associated with the infection or immune disorder.

106. The method of claim 79, wherein the plurality of antigens comprises antigens associated with two or more diseases or disorders.

107. The method of claim 106, wherein the two or more diseases or disorders comprises a SARS-CoV-2 infection, a SARS-CoV-2 variant infection, a non-SARS-CoV-2 coronavirus infection, a non-SARS-CoV-2 viral infection, influenza, or an immune disorder, or any combination thereof.

108. The method of claim 106, wherein the plurality of antigens comprises at least one antigen with high specificity for an immunoglobulin associated with at least one of the two or more diseases or disorders and at least one antigen with high sensitivity for an immunoglobulin associated with at least one of the two or more diseases or disorders.

109. The method of claim 106, further comprising determining, based on the detected changes in resonance wavelength for the plurality of optical ring resonators, an overall sensitivity and specificity for the two or more diseases or disorders.

110. The method of claim 93, wherein the presence of immunoglobulins that are specific for an antigen with high specificity of the plurality of antigens reduces a false positive reading of the infection or immune disorder, or at least one of the two or more diseases or disorders.

111. The method of claim 93, wherein the presence of immunoglobulins that are specific for an antigen with high sensitivity of the plurality of antigens reduces a false negative reading of the infection or immune disorder, or at least one of the two or more diseases or disorders.

112. The method of claim 93, wherein the infection or immune disorder comprises a SARS-CoV-2 infection, and the plurality of antigens comprises at least 1 antigen with a protein sequence unique to SARS-CoV-2, and the plurality of antigens further comprise at least 1 antigen with a protein sequence that is common in Coronaviridae with at least 50% identity.

113. The method of claim 93, wherein the infection or immune disorder comprises a SARS-CoV-2 infection, and the plurality of antigens comprises at least 1 antigen with a protein sequence unique to SARS-CoV-2, and the plurality of antigens further comprise at least 1 antigen with a protein sequence that is associated with a virus that is not SARS-CoV-2.

114. The method of claim 79, wherein the plurality of antigens comprises one or more of SEQ ID NOs: 1-8.

115. The method of claim 79, wherein the plurality of antigens comprises one or more of SEQ ID NOs: 4-8.

116. (canceled)

117. The method of claim 92, wherein determining whether or not the subject has or previously had an infection or immune disorder is performed with a machine learning algorithm.

118. The method of claim 117, wherein the machine learning algorithm is a random forest machine learning algorithm.


SEQUENCE LISTING (8 sequences)

SEQ ID NO: 1 - protein, 1273 aa, Artificial Sequence
  SARS-CoV-2 spike (S) protein sequence
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 222 aa, Artificial Sequence
  SARS-CoV-2 membrane (M) protein sequence
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 3 - protein, 75 aa, Artificial Sequence
  SARS-CoV-2 envelope (E) protein sequence
MYSFVSEETGTLIVNSVLLFLAFVVFLLVTLAILTALRLCAYCCNIVNVSLVKPSFYVYSRVKNLNSSRVPDLLV

SEQ ID NO: 4 - protein, 419 aa, Artificial Sequence
  SARS-CoV-2 nucleocapsid (N) protein sequence
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 5 - protein, 234 aa, Artificial Sequence
  SARS-COV-2(2019-nCoV) Spike Protein (RBD, His Tag)
  10x_His_tag (225)..(234)
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFAHHHHHHHHHH

SEQ ID NO: 6 - protein, 539 aa, Artificial Sequence
  SARS-COV-2(2019-nCoV) Spike Protein (S2 ECD, His Tag)
  10x_His_tag (530)..(539)
SVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPAHHHHHHHHHH

SEQ ID NO: 7 - protein, 688 aa, Artificial Sequence
  SARS-CoV-2 (COVID-19) S1 Protein, His Tag
  linker (671)..(678)
  10x_His_tag (679)..(688)
VNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARGGGSGGGSHHHHHHHHHH

SEQ ID NO: 8 - protein, 1209 aa, Artificial Sequence
  SARS-COV-2(2019-nCoV) Spike Protein (S1+S2 ECD, His Tag)
  10x_His_tag (1200)..(1209)
VNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPAHHHHHHHHHH